(19) United States
(12) Patent Application Publication
(10) Pub. No.: US 2024/0190943 A1
(43) Pub. Date: Jun. 13, 2024

(54) FUSION MOLECULES TARGETING VEGF AND ANGIOPOIETIN AND USES THEREOF

(71) Applicant: HANGZHOU EXEGENESIS BIO LTD., Hangzhou, Zhejiang (CN)

(72) Inventors: Guojie YE, Gainesville, FL (US); Zhenhua WU, Lansdale, PA (US); Lijun WANG, Brisbane, CA (US); Li DAI, Hangzhou, Zhejiang (CN)

(73) Assignee: HANGZHOU EXEGENESIS BIO LTD., Hangzhou, Zhejiang (CN)

(21) Appl. No.: 18/552,653

(22) PCT Filed: Mar. 30, 2022
(86) PCT No.: PCT/CN2022/084074

§ 371 Date: Sep. 26, 2023

(30) Foreign Application Priority Data
Mar. 31, 2021 (WO) PCT/CN2021/084559

(51) Int. Cl. C07K 14/71; A61K 9/00; A61K 38/00; A61P 27/02; C12N 15/86

(52) U.S. Cl. CPC C07K 14/71; A61K 9/0048; A61P 27/02; C12N 15/86; A61K 38/00; C07K 2319/02; C07K 2319/30; C12N 2750/14143

(57) ABSTRACT

A polypeptide comprising two or three VEGF binding domains; and a domain that binds to an angiopoietin, and a gene therapy for delivering such a polypeptide.

### CROSS-REFERENCE TO RELATED APPLICATIONS

This application claims the benefit of PCT Application No. PCT/CN2021/084559, filed Mar. 31, 2021, which is herein incorporated by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file “14652-025-228_SEQ_LISTING.txt” and a creation date of Mar. 18, 2022 and having a size of 217,975 bytes. The sequence listing submitted via EFS-Web is part of the specification and is herein incorporated by reference in its entirety.

### 1. FIELD

The present disclosure relates to fusion molecules (e.g., a polypeptide) that bind to both vascular endothelial growth factor (VEGF) and an angiopoietin (e.g., angiopoietin 1 and angiopoietin 2), gene therapies based on such fusion molecules, and methods of use thereof.

### 2. BACKGROUND

The VEGF/VEGFR and angiopoietin/Tie-2 signaling pathways are important in the process of vascular endothelial growth (angiogenesis) and in the maintenance of angiogenesis associated blood vessels. See Biela and Siemann, Cancer Lett. 380(2): 525-533 (2016). Aberrant angiogenesis is implicated in a number of conditions, such as diabetic retinopathy, psoriasis, exudative or “wet” age-related macular degeneration (“wAMD”), rheumatoid arthritis and other inflammatory diseases, and most cancers. The diseased tissues or tumors associated with these conditions typically express abnormally high levels of VEGF, and show a high degree of vascularization or vascular permeability. For example, wAMD is an angiogenic disease, characterized by choroidal neovascularization in one or both eyes in aging individuals, and is the major cause of blindness. See Gehrs et al., Ann Med. 38(7): 450-471 (2006). A number of therapeutic strategies exist for inhibiting aberrant angiogenesis, targeting VEGF or angiopoietin. See Biela and Siemann, Cancer Lett. 380(2): 525-533 (2016). However, such treatments usually require repeated injections, which can increase the risks of inflammation, infection, and other adverse effects in some patients. Repeated injections also associate with patient compliance and adherence challenges and non-compliance can result in vision loss and deterioration of the eye disease or condition. The rate of non-compliance and non-adherence to treatment regimens that require repeated or frequent trips to medical offices for administration is particularly higher among elderly patients, who are most impacted by AMD. There is a need in the art for improved therapeutic molecules, particularly for use in gene therapies for treating diseases such as wAMD.

### 3. SUMMARY

In one aspect, provided herein is a polypeptide comprising: (i) a first domain that binds to VEGF; (ii) a second domain that binds to VEGF; and (iii) a third domain that binds to an angiopoietin (e.g., angiopoietin 1 and angiopoietin 2) In some embodiments, the third domain is optionally at the N-terminus of the first domain and the second domain.

In some embodiments, the first domain is derived from VEGF receptor-1 (VEGFR-1 or FLT-1). In some embodiments, the first domain comprises domain 2 of VEGFR-1 or a variant thereof. In some embodiments, the first domain comprises or consists of an amino acid sequence of SEQ ID NO: 1. In some embodiments, the first domain comprises or consists of an amino acid sequence having at least 80%, 85%, 90%, 91%, 92%, 93%, 94%, 95%, 96%, 97%, 98%, 99% identify to SEQ ID NO: 1.

In some embodiments, the second domain is derived from VEGF receptor-2 (VEGFR-2 or Flk-1). In some embodiments, the second domain comprises domain 3 of VEGFR-2 or a variant thereof. In some embodiments, the second domain comprises or consists of an amino acid sequence of SEQ ID NO: 2. In some embodiments, the second domain comprises or consists of an amino acid sequence having at least 80%, 85%, 90%, 91%. 92%, 93%, 94%, 95%, 96%, 97%, 98%, 99% identify to SEQ ID NO: 2.

In some embodiments, the third domain (ABD) comprises one or two repeats of an amino acid sequence of SEQ ID NO: 3. In some embodiments, the third domain comprises one or two repeats of an amino acid sequence of SEQ ID NO: 51. In some embodiments, the third domain comprises two repeats of the amino acid sequence of SEQ ID NO: 3. In some embodiments, the third domain comprises two repeats of the amino acid sequence of SEQ ID NO: 51. In some embodiments, the third domain comprises an amino acid sequence of SEQ ID NO: 3 and an amino acid sequence of SEQ ID NO: 51.

In some embodiments, the third domain comprises or consists of an amino acid sequence of SEQ ID NO: 4, or the third domain comprises an amino acid sequence having at least 80%, 85%, 90%, 91%, 92%, 93%, 94%, 95%, 96%, 97%, 98%, 99% identify to SEQ ID NO: 4.

In some embodiments, the third domain comprises or consists of an amino acid sequence of SEQ ID NO: 52, or the third domain comprises an amino acid sequence having at least 80%, 85%, 90%, 91%, 92%, 93%, 94%, 95%, 96%, 97%, 98%, 99% identify to SEQ ID NO: 52.

In some embodiments, the third domain comprises or consists of an amino acid sequence of SEQ ID NO: 53, or the third domain comprises an amino acid sequence having at least 80%, 85%, 90%, 91%, 92%, 93%, 94%, 95%, 96%, 97%, 98%, 99% identify to SEQ ID NO: 53

In some embodiments, the third domain comprises or consists of an amino acid sequence of SEQ ID NO: 54, or the third domain comprises an amino acid sequence having at least 80%, 85%, 90%, 91%, 92%, 93%, 94%, 95%, 96%, 97%, 98%, 99% identify to SEQ ID NO: 54.

In another aspect, provided herein is a polypeptide comprising (i) a first domain that binds to VEGF, the first domain comprising an amino acid sequence having at least 80%, 85%, 90%, 91%, 92%, 93%, 94%, 95%, 96%, 97%, 98%, 99% or 100% identify to SEQ ID NO: 1; (ii) a second domain that binds to VEGF, the second domain comprising an amino acid sequence having at least 80%, 85%, 90%, 91%, 92%, 93%, 94%, 95%, 96%, 97%, 98%, 99% or 100% identify to SEQ ID NO: 2; and (iii) a third domain that binds to an angiopoietin, the third domain comprising two amino acid sequences each comprising an amino acid sequence having at least 80%, 85%, 90%, or 100% identify to SEQ ID NO: 3.

In another aspect, provided herein is a polypeptide comprising (i) a first domain that binds to VEGF, the first domain comprising an amino acid sequence having at least 80%, 85%, 90%, 91%, 92%, 93%, 94%, 95%, 96%, 97%, 98%, 99% or 100% identify to SEQ ID NO: 1; (ii) a second domain that binds to VEGF, the second domain comprising an amino acid sequence having at least 80%, 85%, 90%, 91%, 92%, 93%, 94%, 95%, 96%, 97%, 98%, 99% or 100% identify to SEQ ID NO: 2; and (iii) a third domain that binds to an angiopoietin, the third domain comprising two amino acid sequences each comprising an amino acid sequence having at least 80%, 85%, 90%, or 100% identify to SEQ ID NO: 51.

In another aspect, provided herein is a polypeptide comprising (i) a first domain that binds to VEGF, the first domain comprising an amino acid sequence having at least 80%, 85%, 90%, 91%, 92%, 93%, 94%, 95%, 96%, 97%, 98%, 99% or 100% identify to SEQ ID NO: 1; (ii) a second domain that binds to VEGF, the second domain comprising an amino acid sequence having at least 80%, 85%, 90%, 91%, 92%, 93%, 94%, 95%, 96%, 97%, 98%, 99% or 100% identify to SEQ ID NO: 2; and (iii) a third domain that binds to an angiopoietin, the third domain comprising one amino acid sequence having at least 80%, 85%, 90%, or 100% identify to SEQ ID NO: 3 and one amino acid sequence having at least 80%, 85%, 90%, or 100% identify to SEQ ID NO: 51.

In some embodiments, the polypeptide further comprises an Fc region of an antibody. In some embodiments, the Fc region comprises an amino acid sequence of SEQ ID NO: 5.

In some embodiments, the polypeptide further comprises a signal peptide. In some embodiments, the signal peptide comprises an amino acid sequence of SEQ ID NO: 6.

In some embodiments, the polypeptide further comprises one or more linkers.

In some embodiments, provided herein is a polypeptide comprising an amino acid sequence of SEQ ID NO: 7, SEQ ID NO: 8, SEQ ID NO: 9, or SEQ ID NO: 10, or an amino acid sequence having at least 80%, 85%, 90%, 91%, 92%, 93%, 94%, 95%, 96%, 97%, 98%, 99% identify to SEQ ID NO: 7, SEQ ID NO: 8, SEQ ID NO: 9, or SEQ ID NO: 10.

In some embodiments, provided herein is a polypeptide comprising an amino acid sequence of SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, or SEQ ID NO: 66, or an amino acid sequence having at least 80%, 85%, 90%, 91%, 92%, 93%, 94%, 95%, 96%, 97%, 98%, 99% identify to SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, or SEQ ID NO: 66.

In some embodiments, the polypeptide provided herein further comprises a VEGFC binding domain. In some embodiments, the VEGFC binding domain is derived from VEGFR-2. In other embodiments, the VEGFC binding domain is derived from VEGFR-3.

In some embodiments, the VEGFC binding domain comprises an amino acid sequence having at least 80%, 85%, 90%, 91%, 92%, 93%, 94%, 95%, 96%, 97%, 98%, 99% or 100% identify to SEQ ID NO: 55.

In some embodiments, the VEGFC binding domain comprises an amino acid sequence having at least 80%, 85%, 90%, 91%, 92%, 93%, 94%, 95%, 96%, 97%, 98%, 99% or 100% identify to SEQ ID NO: 56.

In some embodiments, the VEGFC binding domain comprises an amino acid sequence having at least 80%, 85%, 90%, 91%, 92%, 93%, 94%, 95%, 96%, 97%, 98%, 99% or 100% identify to SEQ ID NO: 57.

In some embodiments, the polypeptide is genetically fused or chemically conjugated to an agent.

In another aspect, provided herein is an isolated nucleic acid comprising a nucleic acid sequence encoding the polypeptide provided herein.

In yet another aspect, provided herein is a vector comprising the isolated nucleic acid provided herein. In some embodiments, the vector is a viral vector. In some embodiments, the viral vector is an adeno-associated virus (AAV) vector. In some embodiments, the AAV vector is derived from AAV1, AAV2, AAV2i8, AAV3, AAV3-B. AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a combination or variant thereof. In some embodiments, the vector is a recombinant AAV2 (rAAV2) vector, a recombinant AAV8 (rAAV8) vector or a variant thereof.

In another aspect, provided herein is a recombinant AAV (rAAV) vector comprising a nucleic acid encoding a polypeptide wherein the polypeptide comprises (i) a first domain derived from VEGFR-1; (ii) a second domain derived from VEGFR-2; and (iii) a third domain that is capable of binding to an angiopoietin (e.g., angiopoietin 1 and angiopoietin 2), wherein the rAAV vector comprises an inverted terminal repeat (ITR) from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a combination or variant thereof. In some embodiments, the ITR is from AAV2. In other embodiments, the ITR is from AAV8. In some embodiments, the third domain is at N-terminus of the first domain and the second domain.

In yet another aspect, provided herein is a recombinant AAV (rAAV) vector comprising a nucleic acid encoding a polypeptide wherein the polypeptide comprises (i) a first domain derived from VEGFR-1; (ii) a second domain derived from VEGFR-2; (iii) a third domain that is capable of binding to an angiopoietin; and (iv) a fourth domain that is capable of binding to VEGFC, wherein the rAAV vector comprises an inverted terminal repeat (ITR) from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, or AAV44-9. In some embodiments, the ITR is from AAV2. In other embodiments, the ITR is from AAV8. In some embodiments, the third domain is at N-terminus of the first domain and the second domain.

In some embodiments, provided herein is a vector comprising a nucleic acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23, or a nucleic acid sequence having at least 80%, 85%, 90%, 91%, 92%, 93%, 94%, 95%, 96%, 97%, 98%, 99% identify to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23.

In some embodiments, provided herein is a vector comprising a nucleic acid sequence of SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75, or a nucleic acid sequence having at least 80%, 85%, 90%, 91%, 92%, 93%, 94%, 95%, 96%, 97%, 98%, 99% identify to SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75.

In yet another aspect, provided herein is a recombinant AAV (rAAV) particle comprising (a) a nucleic acid encoding a polypeptide wherein the polypeptide comprises (i) a first domain derived from VEGFR-1; (ii) a second domain derived from VEGFR-2; and (iii) a third domain that is capable of binding to an angiopoietin (e.g., angiopoietin 1 and angiopoietin 2); and (b) a capsid protein of AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a variant thereof. In some embodiments, the third domain is at N-terminus of the first domain and the second domain. In some embodiments, the capsid protein is an AAV2 capsid protein. In some embodiments, the capsid protein is an AAV8 capsid protein. In some embodiments, the capsid protein is a variant of an AAV2 capsid protein comprising an amino acid sequence of SEQ ID NO: 48, wherein the variant comprises the amino acid substitutions of Y444F, R487G, T491V, Y500F, R585S, R588T, and Y730F of capsid protein VP1 of AAV2.

In yet another aspect, provided herein is a recombinant AAV (rAAV) particle comprising (a) a nucleic acid encoding a polypeptide wherein the polypeptide comprises (i) a first domain derived from VEGFR-1; (ii) a second domain derived from VEGFR-2; and (iii) a third domain that is capable of binding to an angiopoietin; and (iv) a fourth domain capable of binding to VEGFC; and (b) a capsid protein of AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a variant thereof.

A pharmaceutical composition comprising the polypeptide, the vector or rAAV vector, or the rAAV particle provided herein, and a pharmaceutically acceptable excipient.

In yet another aspect, provided herein is a method of treating a disease or disorder in a subject, comprising administering to the subject the polypeptide, the vector, or the pharmaceutical composition provided herein. In some embodiments, the disease or disorder is an angiogenic or neovascular disease or disorder. In some embodiments, the disease or disorder is an inflammatory disease, ocular disease, autoimmune disease, or cancer. In some embodiments, the disease or disorder is an eye disease or disorder. In some embodiments, the eye disease or disorder is selected from a group consisting of uveitis, retinitis pigmentosa, neovascular glaucoma, diabetic retinopathy (DR) (including proliferative diabetic retinopathy), ischemic retinopathy, intraocular neovascularization, age-related macular degeneration (AMD), retinal neovascularization, diabetic macular edema (DME), diabetic retina ischemia, diabetic retinal edema, retinal vein occlusion (including central retinal vein occlusion and branched retinal vein occlusion), macular edema, and macular edema following retinal vein occlusion (RVO). In some embodiments, the disease or disorder is age-related macular degeneration (AMD). In some embodiments, the AMD is wet AMD (wAMD).

In some embodiments, the method comprises administering by intravitreal or subretinal injection into an eye of the subject.

### 4. BRIEF DESCRIPTION OF THE FIGURES

FIG. 1 illustrates the exemplary fusion protein constructs provided herein (Flt1 signal: the signal peptide sequence of VEGFR1 (Flt1); VEGFR1 D2: the IgG-like domain 2 of VEGF receptor 1; VEGFR2 D3: the IgG like domain 3 of VEGF receptor 2; Ang BD: the angiopoietin binding domain; IgG Fc: Fc fragment of human IgG).

FIG. 2 illustrates the exemplary rAAV vectors provided herein. TR represents AAV2 inverted terminal repeats, CBA is the 1.68 kb chicken beta-actin promoter, CB is the 0.78 kb small chicken beta-actin promoter, D2 represents the IgG-like domain 2 of VEGFR-1, D3 represents the IgG like domain 3 of VEGFR-2, ABD (or Ang BD) represents the angiopoietin binding domain, IgG Fc is Fc fragment of human IgG, Fc-Hinger is the N-terminal 21 amino acid of the Fc plus a 6 amino acid GS linker, WPRE represents woodchuck hepatitis virus posttranscriptional regulatory element (600 bp), mini mWPRE is the WPRE (240 bp), SV40 pA is simian virus 40 polyadenylation signal, and bGHpA is the bovine growth hormone polyadenylation signal.

FIGS. 3A-3B illustrate the experimental designs and assays for testing expression and binding affinity of the constructs provided herein.

FIGS. 4A-4B show that position of Ang BD at C-terminus did not affect the binding ability to rVEGF, but reduced the transgene expression levels.

FIGS. 5A-5B show that position of Ang BD in the middle between VEGF binding domains and Fc region did not affect the binding ability to rVEGF and transgene expression.

FIGS. 6A-6B show that position of Ang BD in the middle between VEGF binding domains and Fc region inhibited the binding ability to Ang2. In the upper panel of FIG. 6B, the right bar of each of the two pairs represents the binding for EXG102-09, and the left bar of each of the two pairs represents the binding for FXG102-04

FIGS. 7A-7D show that the constructs that have Ang BD at the N-terminus bind strongly to both VEGF and Ang2, while maintaining high expression level of the transgene. In FIG. 7D, for each EXG construct, the left bar represents binding to rVEGF, the middle bar represents binding to Ang2, and the right bar represents the protein expression level.

FIG. 8 illustrates schematic design of the additional exemplary fusion protein constructs comprising a VEGF-C binding domain (Trap-C1, C2, or C3) and angiopoitin binding domain (ABD or ABD2) provided herein. TR, Inverted terminal repeat sequence; CBA, chimeric CMV-chicken ß-actin promoter; ABD, angiopoietin binding domain; D2, the IgG-like domain 2 of VEGF receptor 1; D3, the IgG like domain 3 of VEGF receptor 2; Trap C, VEGFC binding domain; Fc, fragment crystallizable of IgG1; bGHpA, bovine growth hormone polyadenylation signal; VEGF, vascular endothelial growth factor; IgG1, immunoglobulin G1.

FIGS. 9A-91 illustrate EXG102-24, EXG102-25, EXG102-26, EXG102-27, EXG102-28, EXG102-29, EXG102-30, EXG102-31, and EXG102-32, respectively. The sequences are indicated and numbered (the fusion proteins illustrated in FIGS. 9A-91 comprise SEQ ID NOs: 58-66, respectively). Signal peptide, ABD, D2, D3, Trap C, and Fc constructs are also indicated in the figure. The GS linker was highlighted. TR, Inverted terminal repeat sequence; CBA, chimeric CMV-chicken ß-actin promoter; ABD, angiopoietin binding domain; D2, the IgG-like domain 2 of VEGF receptor 1; D3, the IgG like domain 3 of VEGF receptor 2; Trap C, VEGFC binding domain; Fc, fragment crystallizable of IgG1; bGHpA, bovine growth hormone polyadenylation signal; VEGF, vascular endothelial growth factor; IgG1, immunoglobulin G1.

FIGS. 10A-10F show that constructs containing ABD2 are comparable to the constructs with ABD domain in protein expression, VEGF-A binding or Ang2 binding.

FIG. 11 shows the FFA mean score in laser-induced choroidal neovascularization (CNV) mouse model. The FFA mean score was measured in eyes treated with AAV-GFP (negative control), EXG102-02 (positive control), EXG102-04, EXG102-09, EXG102-10, or EXG102-11 on days 29 and 36 post injection. The angiograms were graded as follows: score 0, no staining; score 1, slightly stained; score 2, moderately stained; and score 3, strongly stained. The two constructs having the highest CNV inhibition are highlighted by arrowheads. Bars present Mean with SD.

FIG. 12 shows the ratio of Grade 3 CNV lesions in laser-induced CNV mouse model. The FFA mean score was measured in eyes treated with AAV-GFP (negative control), EXG102-02 (positive control), EXG102-04, EXG102-09, EXG102-10, or EXG102-11. The angiograms were graded as follows: score 0, no staining; score 1, slightly stained; score 2, moderately stained; and score 3, strongly stained. Bars present Mean with SD.

FIGS. 13A-13C shows in vitro VEGF-A, Ang2, or VEGF-C binding affinity of transgene product expressed from HEK293T cells that were transfected with plasmid DNA. HEK293T cells were transfected with plasmid DNA of pEXG102-02, pEXG102-30 or pEXG102-31 and the target protein expressed were affinity purified from the cell lysate. Binding capability of EXG102-02, EXG102-30 or EXG102-31 to VEGF-A, Ang2, or VEGF-C was measured by ELISA respectively. Bars present Mean with SD.

FIG. 14 shows the FFA mean score in laser-induced CNV mouse model. The FFA mean score was measured in eyes treated with vehicle control (black), EXG102-02 (medium grey), EXG102-30 (dark grey), or EXG102-31(light grey). Animals were given intraperitoneal injection of Fluorescein Sodium (100 mg/ml, 30 μL/animal) before fluorescein angiography. The FFA images were taken 3 minutes post injection. The angiograms were graded as follows: score 0, no staining; score 1, slightly stained; score 2, moderately stained; and score 3, strongly stained. Rate of Score 3 lesions and the mean score were calculated. Bars present Mean with SD.

FIG. 15 shows the ratio of Grade 3 CNV lesions in laser-induced CNV mouse model. The ratio of Grade 3 CNV lesions were measured in eyes treated with vehicle control, EXG102-02, EXG102-30, or EXG102-31. Animals were given intraperitoneal injection of Fluorescein Sodium (100 mg/ml, 30 L/animal) before fluorescein angiography. The FFA images were taken 3 minutes post injection. The angiograms were graded as follows: score 0, no staining; score 1, slightly stained; score 2, moderately stained; and score 3, strongly stained. Rate of Score 3 lesions and the mean score were calculated. Bars present Mean with SD.

### 5. DETAILED DESCRIPTION

The present disclosure is based in part on the novel fusion proteins that comprise domains from VEGFR-1, VEGFR-2, and/or VEGFR-3 and an angiopoietin binding domain, AAV vector comprising a nucleic acid encoding such fusion proteins, and improved properties thereof.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between two molecules is the affinity of one molecule to the other. The ratio of dissociation rate (k_{off}) to association rate (kₒₙ) of a binding molecule to another molecule (k_{off}/kₒₙ) is the dissociation constant K_{D}, which is inversely related to affinity. The lower the K_{D} value, the higher the affinity. The value of K_{D} varies for different complexes depends on both kₒₙ and k_{off}. The dissociation constant K_{D} can be determined using any method provided herein or any other method well known to those skilled in the art.

In connection with the binding molecules described herein terms such as “bind to,” “that specifically bind to,” and analogous terms are also used interchangeably herein and refer to that binding domains specifically bind to another molecule, such as a polypeptide. A binding molecule or binding domain that binds to or specifically binds to another molecule can be identified, for example, by immunoassays, Octet®, Biacore®, or other techniques known to those of skill in the art. In some embodiments, a binding molecule or binding domain binds to or specifically binds to a molecule when it binds to the molecule with higher affinity than to any cross-reactive molecules as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or binding domain to a “non-target” protein is less than about 10% of the binding of the binding molecule or binding domain to its particular target, for example, as determined by fluorescence activated cell sorting (FACS) analysis or RIA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. A binding molecule or binding domain that binds to a molecule includes one that is capable of binding the molecule with sufficient affinity such that the binding molecule is useful, for example, as a diagnostic or therapeutic agent in targeting the molecule. In certain embodiments, a binding molecule or binding domain that binds to a target molecule has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM.

“Binding affinity” generally refers to the strength of the sum total of noncovalent interactions between a single binding site of a molecule and its binding partner. Unless indicated otherwise, as used herein, “binding affinity” refers to intrinsic binding affinity which reflects a 1:1 interaction between members of a binding pair. The affinity of a binding molecule X for its binding partner Y can generally be represented by the dissociation constant (K_{D}). Affinity can be measured by common methods known in the art, including those described herein. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} or K_{D} value may also be measured by using biolayer interferometry (BLI) or surface plasmon resonance (SPR) assays by Octet®, using, for example, an Octet®Red96 system, or by Biacore®, using, for example, a Biacore®TM-2000 or a Biacore® TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)2 fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the polypeptides have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is usually substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a fusion protein as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a polypeptide described herein is a nucleic acid molecule that is identified and separated from at least one contaminant nucleic acid molecule with which it is ordinarily associated in the environment in which it was produced.

The term “variant” as used herein in connection with a polypeptide or a protein refers to a polypeptide having certain percent sequence identity to a reference polypeptide, for example, having at least about 80% amino acid sequence identity with a reference polypeptide, e.g., the corresponding full-length native sequence. Such polypeptide variants include, for instance, polypeptides wherein one or more amino acid residues are added, or deleted. In embodiments, a variant has at least about 80% amino acid sequence identity, at least about 81% amino acid sequence identity, at least about 82% amino acid sequence identity, at least about 83% amino acid sequence identity, at least about 84% amino acid sequence identity, at least about 85% amino acid sequence identity, at least about 86% amino acid sequence identity, at least about 87% amino acid sequence identity, at least about 88% amino acid sequence identity, at least about 89% amino acid sequence identity, at least about 90% amino acid sequence identity, alternatively at least about 91% amino acid sequence identity, at least about 92% amino acid sequence identity, at least about 93% amino acid sequence identity, at least about 94% amino acid sequence identity, at least about 95% amino acid sequence identity, at least about 96% amino acid sequence identity, at least about 97% amino acid sequence identity, at least about 98% amino acid sequence identity, or at least about 99% amino acid sequence identity to the reference polypeptide, e.g., the corresponding full-length native sequence. In embodiments, variant polypeptides are at least about 10 amino acids in length, at least about 20 amino acids in length, at least about 30 amino acids in length, at least about 40 amino acids in length, at least about 50 amino acids in length, at least about 60 amino acids in length, at least about 70 amino acids in length, at least about 80 amino acids in length, at least about 90 amino acids in length, at least about 100 amino acids in length, at least about 150 amino acids in length, at least about 200 amino acids in length, at least about 300 amino acids in length, or more. Variants include substitutions that are conservative or non-conservative in nature. For example, the polypeptide of interest may include up to about 5-10 conservative or non-conservative amino acid substitutions, or even up to about 15-25 or 50 conservative or non-conservative amino acid substitutions, or any number between 5-50.

The term “homology” as used herein refers to the percent identity between two polynucleotide or two polypeptide moieties. Two DNA, or two polypeptide sequences are “substantially homologous” to each other when the sequences exhibit at least about 50%, at least about 75%, at least about 80%-85%, at least about 90%, at least about 95%-98% sequence identity, at least about 99%, or any percent therebetween over a defined length of the molecules. As used herein, substantially homologous also refers to sequences showing complete identity to the specified DNA or polypeptide sequence.

The term “identity” as used herein refers to an exact nucleotide-to-nucleotide or amino acid-to-amino acid correspondence of two polynucleotides or polypeptide sequences, respectively. Methods for determining percent identity are well known in the art. For example, percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions. Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code-standard; filter-none; strand-both; cutoff-60; expect-10; Matrix-BLOSUM62; Descriptions=50 sequences; sort by=HIGH SCORE; Databases=non-redundant, GenBank+EMBL+DDBJ+PDB+GenBank CDS translations-Swiss protein+Spupdate+PIR. Details of these programs are well known in the art. Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The term “vector” as used herein refers to a substance that is used to carry or include a nucleic acid sequence, for example, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed both nucleic acid molecules can be inserted, for example, into a single expression vector or in separate expression vectors. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. The term “vector” includes cloning and expression vehicles, as well as viral vectors. In certain embodiments, the vector provided herein is a recombinant AAV vector.

The term “recombinant AAV vector (rAAV vector)” as used herein refers to a polynucleotide vector comprising a nucleic acid sequence from an AAV and one or more heterologous sequences (i.e., nucleic acid sequence not of AAV origin). In some embodiments, the one or more heterologous sequences are flanked by at least one, in certain embodiments two, AAV inverted terminal repeat sequences (ITRs). In some embodiments, such rAAV vectors can be replicated and packaged into infectious viral capsid particles, e.g., when present in a host cell that has been infected with a suitable helper virus (or that is expressing suitable helper functions) and that is expressing AAV rep and cap gene products (i.e., AAV Rep and Cap proteins). An rAAV vector may be incorporated into a larger polynucleotide (e.g., in a chromosome or in another vector such as a plasmid used for cloning or transfection), and can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral capsid particle, particularly an AAV particle. An rAAV vector can be packaged into an AAV capsid to generate a “recombinant adeno-associated viral capsid particle (rAAV particle).”

The term “heterologous” as used herein in connection with nucleic acid sequences such as coding sequences and control sequences, refers to sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene).

The term “flanked” as used herein with respect to a sequence that is flanked by other elements, indicates the presence of one or more the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence (e.g., a transgene) that is “flanked” by two other elements (e.g., TRs) indicates that one element is located 5′ to the sequence and the other is located 3′ to the sequence; however, there may be intervening sequences therebetween.

The term “inverted terminal repeat” or “ITR” sequence as used herein refers to relatively short sequences found at the termini of viral genomes which are in opposite orientation. An “AAV inverted terminal repeat (ITR)” sequence is well known in the art, and is usually an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “coding sequence” or a sequence which “encodes” a selected polypeptide, is a nucleic acid molecule which is transcribed (in the case of DNA) and translated (in the case of mRNA) into a polypeptide when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “promoter” as used herein in its ordinary sense refers to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters.”

The term “transgene” as used herein in a broad sense means any heterologous nucleotide sequence incorporated in a viral vector, e.g., for expression in a target cell and it can be associated with expression control sequences, such as promoters. It is appreciated by those of skill in the art that expression control sequences will be selected based on ability to promote expression of the transgene in the target cell. An example of a transgene is a nucleic acid encoding a therapeutic polypeptide or a detectable marker.

The term “AAV capsid” or “AAV capsid protein” or “AAV cap” as used herein refers to a protein encoded by an AAV capsid (cap) gene (e.g., VPI, VP2, and VP3) or a variant thereof. For example, the term includes but not limited to a capsid protein derived from any AAV serotype such as AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV-2/1, AAV 2/6, AAV 2/7, AAV 2/8, AAV 2/9, AAV LK03, AAVrh10, AAVrh74, AAV44-9, or a variant thereof. The term also includes a capsid protein expressed by or derived from a recombinant AAV such as a chimeric AAV.

The term “AAV capsid particle” or “AAV particle” as used herein includes at least one AAV capsid protein (e.g., a VPI protein, a VP2 protein, a VP3 protein, or variant thereof) and optionally encapsulates a nucleic acid from an AAV genome or a nucleic acid derived from an AAV genome.

The term “serotype” used with respect to vector or virus capsid is defined by a distinct immunological profile based on the capsid protein sequences and capsid structure.

The term “chimeric” as used herein means, with respect to a viral capsid or particle, that the capsid or particle includes sequences from different parvoviruses, preferably different AAV serotypes, as described in Rabinowitz et al., U.S. Pat. No. 6,491,907 the disclosure of which is incorporated in its entirety herein by reference.

The term “recombinant” means a genetic entity distinct from that generally found in nature. As applied to a polynucleotide or gene, this means that the polynucleotide is the product of various combinations of cloning, restriction and/or ligation steps, and other procedures that result in the production of a construct that is distinct from a polynucleotide found in nature.

The term “recombinant virus” as used herein refers to a virus that has been genetically altered, e.g., by the addition or insertion of a heterologous nucleic acid construct into the particle. For example, the term “recombinant AAV particle” or “rAAV” as used herein refers to an AAV that has been genetically altered, e.g., by the deletion or other mutation of an endogenous AAV gene and/or the addition or insertion of a heterologous nucleic acid construct into the polynucleotide of the AAV particle.

The term “transfected” or “transformed” or “transduced” as used herein refers to a process by which exogenous nucleic acid is transferred or introduced into a host cell. A “transfected” or “transformed” or “transduced” cell is one which has been transfected, transformed or transduced with exogenous nucleic acid. For example, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous molecules into suitable host cells. “Transduction” of a cell by a virus means that there is transfer of a nucleic acid such as DNA or RNA from the virus particle to the cell.

The term “host cell” as used herein refers to a particular cell that may be transfected with a nucleic acid molecule and the progeny or potential progeny of such a cell. Host cells may be bacterial cells, yeast cells, insect cells or mammalian cell.

The term “purified” refers to isolation of a substance (compound, polynucleotide, protein, polypeptide, polypeptide composition) such that the substance of interest comprises the majority percent of the sample in which it resides. Typically in a sample a substantially purified component comprises 50%, 80%-85%, 90-99%, such as at least 90%, 91%, 92%, 93%, 94%, 95%, 96%, 97%, 98%, 99% of the sample. Techniques for purifying polynucleotides and polypeptides of interest are well-known in the art and include, for example, ion-exchange chromatography, affinity chromatography and sedimentation according to density.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease. “Treatment” or “treating” includes: (1) preventing the disease, i.e., preventing the development of the disease or causing the disease to occur with less intensity in a subject that may be exposed to or predisposed to the disease but does not yet experience or display symptoms of the disease, (2) inhibiting the disease, i.e., arresting the development, preventing or retarding progression, or reversing the disease state, (3) relieving symptoms of the disease i.e., decreasing the number of symptoms experienced by the subject, and (4) reducing, preventing or retarding progression of the disease or a symptom thereof. The terms “prevent,” “preventing,” and “prevention” refer to reducing the likelihood of the onset (or recurrence) of a disease, disorder, condition, or associated symptom(s).

As used herein, “administer”, “administration”, or “administering” refers to the act of injecting or otherwise physically delivering a substance (e.g., a conjugate or pharmaceutical composition provided herein) to a subject or a patient (e.g., human), such as by oral, mucosal, topical, intradermal, parenteral, intravenous, intravitreal, intraarticular, subretinal, intramuscular, intrathecal delivery and/or any other method of physical delivery described herein or known in the art. In a particular embodiment, administration is by intravenous infusion. A conjugate or a composition provided herein may be delivered systemically or to a specific tissue.

As used herein, the terms “effective amount” or “therapeutically effective amount” refer to an amount of a therapeutic (e.g., a conjugate or pharmaceutical composition provided herein) which is sufficient to treat, diagnose, prevent, delay the onset of, reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy. In some embodiments, “effective amount” as used herein also refers to the amount of a conjugate described herein to achieve a specified result. As used herein, the terms “subject” and “patient” are used interchangeably.

As used herein, a subject is a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

As used herein, the terms “therapies” and “therapy” can refer to any protocol(s), method(s), compositions, formulations, and/or agent(s) that can be used in the prevention, treatment, management, or amelioration of a disease or disorder or symptom thereof (e.g., a disease or disorder provided herein or one or more symptoms or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a disease or disorder or one or more symptoms thereof. In certain embodiments, the term “therapy” refers to a therapy other than a conjugate described herein or pharmaceutical composition thereof.

As used herein, the term “disease or disorder associated with angiogenesis” refers to a disease or disorder that involves angiogenesis (including abnormal angiogenesis), e.g., either as a symptom or direct or indirect cause. The term includes the disease or disorder the development of which involves angiogenesis, including, e.g., cancer and eye diseases or disorders.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Fusion Proteins and Variants Thereof

### 5.2.1. Multispecific Fusion Proteins

In one aspect, provided herein are multispecific fusion proteins (or polypeptides) comprising multiple VEGF binding domains (e.g., two or three VEGF binding domains) and a domain that binds to an angiopoietin (e.g., angiopoietin 1 and angiopoietin 2). In some embodiments, the fusion protein provided herein comprises at least three binding domains—a first domain that binds to VEGF, a second domain that binds to VEGF, and a third domain that binds to an angiopoietin (e.g., angiopoietin 1 and angiopoietin 2). In some embodiments, the multispecific fusion protein further comprises a fourth domain that binds to VEGF, and thus provided herein in some embodiments is a fusion protein comprising at least four binding domains—a first domain that binds to VEGF, a second domain that binds to VEGF, a third domain that binds to an angiopoietin (e.g., angiopoietin 1 and angiopoietin 2), and a fourth domain that binds to VEGF (more specifically VEGFC).

Vascular endothelial growth factor (VEGF) belongs to the PDGF supergene family characterized by 8 conserved cysteines and functions as a homodimer structure, and is produced by many cell types including tumor cells, macrophages, platelets, keratinocytes, and renal mesangial cells. VEGF-A regulates angiogenesis and vascular permeability by activating two receptors, VEGFR-1 (Flt-1) and VEGFR-2 (KDR/Flk1). In addition to its functions in the vascular system, VEGF plays a role in normal physiological functions such as bone formation, hematopoiesis, wound healing, and development. Formation of new blood vessels, caused for example by the overproduction of growth factors such as VEGF, is a key component of diseases like tumor growth, age-related macular degeneration (AMD) and proliferative diabetic retinopathy (PDR). The VEGF-VEGFR system is an important target for anti-angiogenic therapy in cancer and is also an attractive system for pro-angiogenic therapy in the treatment of neuronal degeneration and ischemic diseases. Shibuya, Genes Cancer, 2(12): 1097-1105 (2011). Binding of VEGF-C to VEGFR-3 is involved and/or responsible for most of the biological effects of VEGFR-3. The discovery of a soluble form of VEGFR-3 (sVEGFR-3) and experiments on transgenic mice expressing this gene led to the conclusion that sVEGFR-3 inhibits the development of lymphatic vessels and induces edema, inhibiting the signals mediated by VEGF-C and VEGF-D.

Angiopoietins are a family of growth factors that includes the glycoproteins angiopoietin 1 (ANGPT1 or Ang1) and angiopoietin 2 (ANGPT2 or Ang2) and the orthologs 3 (in the mouse) and 4 (in human). Angiopoietins are involved in embryonic vascular development. Angiopoietin 1 is expressed by numerous cell types while angiopoietin 2 is mostly limited to endothelial cells. Both act on the TIE2 receptor tyrosine kinase that is found predominantly on endothelial cells and hematopoietic stem cells. These proteins are important modulators of angiogenesis and maintenance of vascular integrity. “An angiopoietin” as used herein refers any angiopoietin peptide, including angiopoietin 1 and angiopoietin 2; and a “domain that binds to an angiopoietin” refers to a domain that binds to angiopoietin 1 and/or angiopoietin 2.

In some embodiments, the first and the second domains provided herein bind to human VEGF. In some embodiments, the third domain provided herein binds to a human angiopoietin (e.g., angiopoietin 1 and angiopoietin 2). In some embodiments, the fourth domain provided herein binds to human VEGF (e.g., VEGFC).

In some embodiments, the fusion proteins provided herein modulates one or more VEGF activities. In some embodiments, the fusion proteins provided herein modulates one or more angiopoietin (e.g., angiopoietin 1 and angiopoietin 2) activities.

In some embodiments, the first domain provided herein binds to VEGF (e.g., human VEGF) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸M to 10⁻¹³ M, e.g., from 10⁻⁹M to 10⁻¹³ M).

In some embodiments, the second domain provided herein binds to VEGF (e.g., human VEGF) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹M to 10⁻¹³ M).

In some embodiments, the third domain provided herein binds to an angiopoietin (e.g., angiopoietin 1 and angiopoietin 2) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

In some embodiments, the fourth domain provided herein binds to VEGF (e.g., human VEGFC) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹M to 10⁻¹³ M).

K_{D} can be measured using known technologies in the art as well as the methods described in Section 5.1 above.

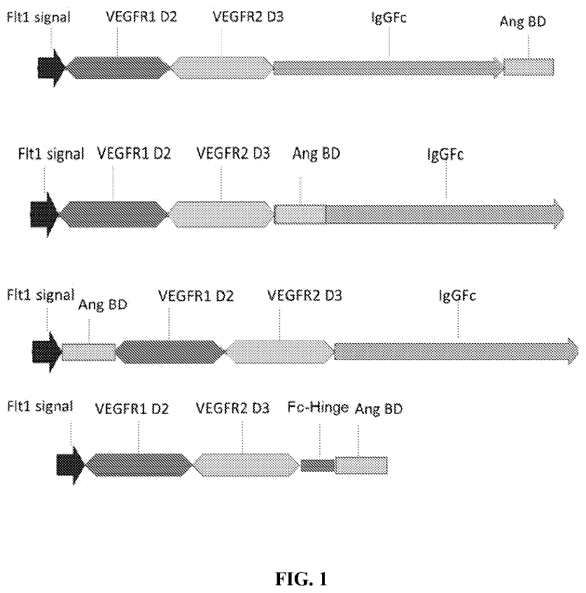

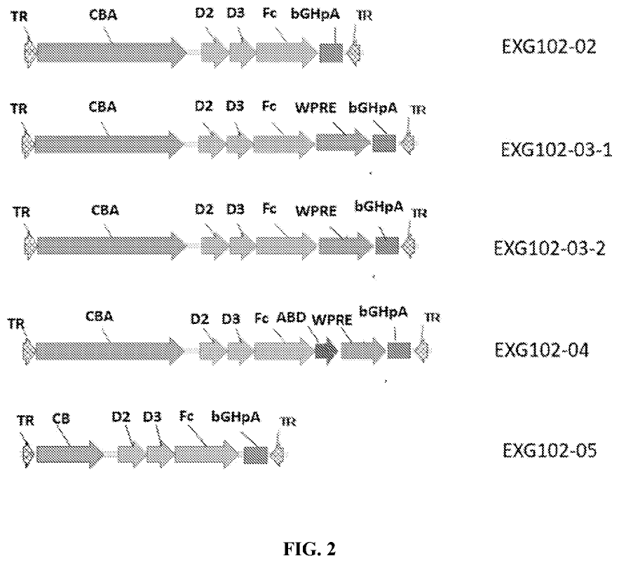

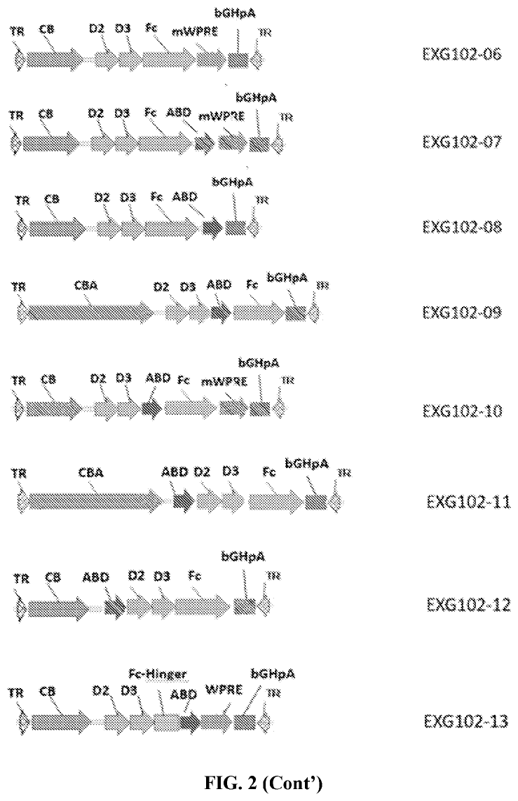

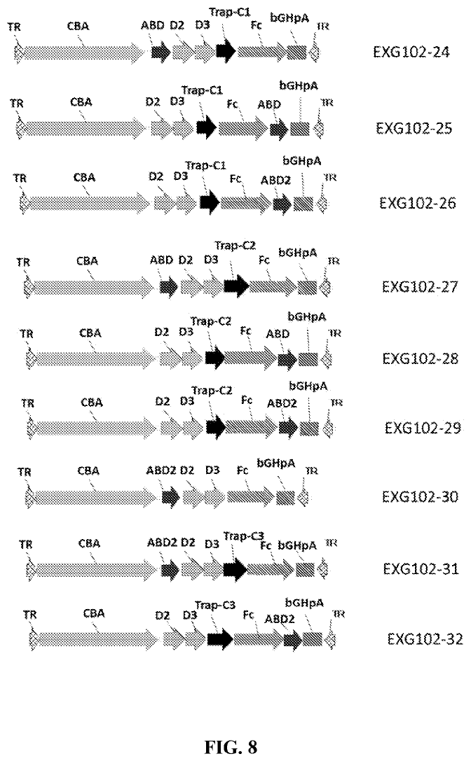

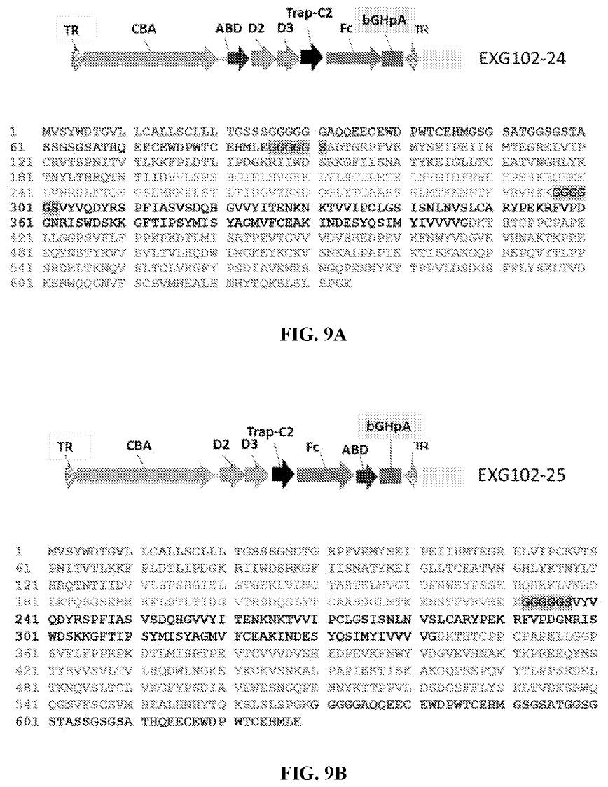

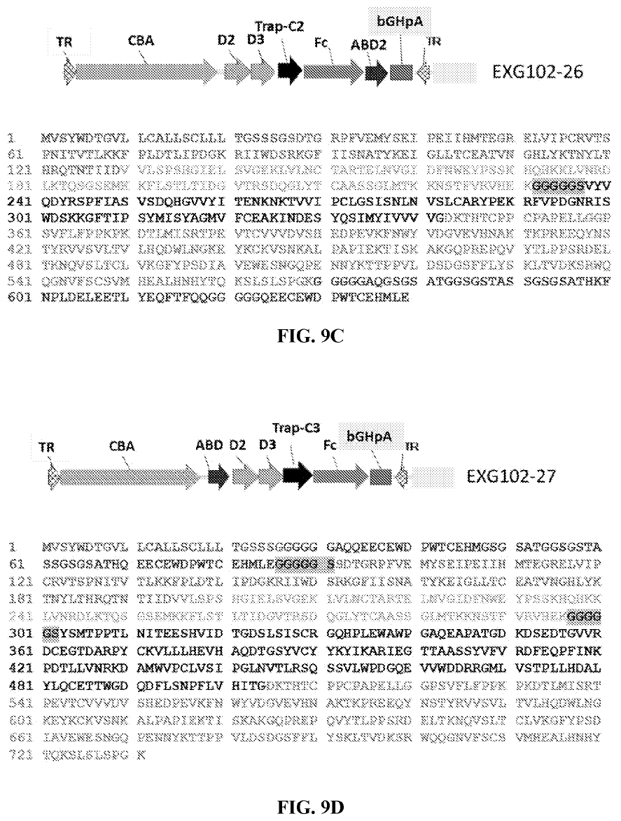

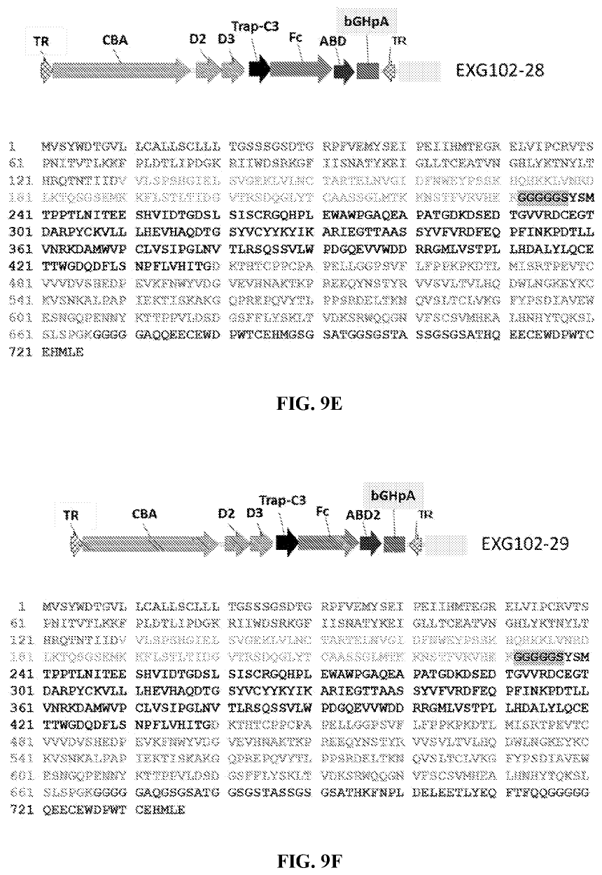

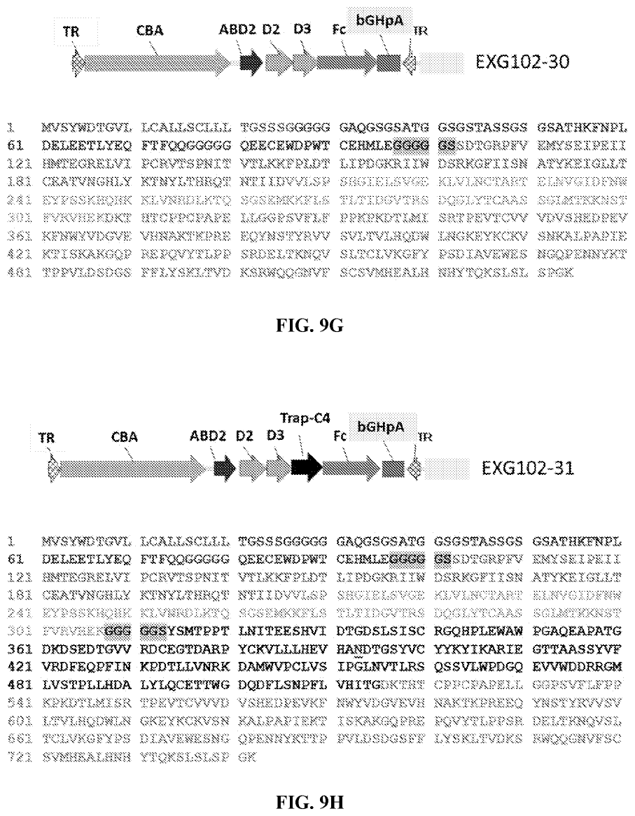

In some embodiments, the first domain is derived from VEGF receptor-1 (VEGFR-1 or FLT-1). In some embodiments, the first domain comprises IgG-like domain 2 (or domain 2 or D2) of VEGFR-1 or a variant thereof. Thus, this domain is referred to D2 in the present figures, e.g., in FIG. 1, FIG. 2, FIG. 8 and FIGS. 9A-9I. In some embodiments, the first domain comprises or consists of an amino acid sequence of SEQ ID NO: 1. In some embodiments, the first domain comprises or consists of an amino acid sequence having at least 80%, 85%, 90%, 91%, 92%, 93%, 94%, 95%, 96%, 97%, 98%, 99% identify to SEQ ID NO: 1.

In some embodiments, the second domain is derived from VEGF receptor-2 (VEGFR-2 or Flk-1). In some embodiments, the second domain comprises IgG-like domain 3 (or domain 3 or D3) of VEGFR-2 or a variant thereof. Thus, this domain is referred to D3 in the present figures, e.g., in FIG. 1, FIG. 2, FIG. 8 and FIGS. 9A-9I. In some embodiments, the second domain comprises or consists of an amino acid sequence of SEQ ID NO: 2. In some embodiments, the second domain comprises or consists of an amino acid sequence having at least 80%, 85%, 90%, 91%, 92%, 93%, 94%, 95%, 96%, 97%, 98%, 99% identify to SEQ ID NO: 2.

In some embodiments, the third domain (ABD) comprises one or more repeat(s) of an amino acid sequence of SEQ ID NO: 3. In some embodiments, the third domain comprises one or more repeat(s) of an amino acid sequence of SEQ ID NO: 51. In some embodiments, the third domain comprises two repeats of the amino acid sequence of SEQ ID NO: 3. In some embodiments, the third domain comprises two repeats of the amino acid sequence of SEQ ID NO: 51. In other embodiments, the third domain comprises an amino acid sequence of SEQ ID NO: 3 and an amino acid sequence of SEQ ID NO: 51. In case wherein the ABD in the present fusion protein comprises an amino acid sequence of SEQ ID NO: 3 and an amino acid sequence of SEQ ID NO: 51, the two sequences can be in any order, for example, the amino acid sequence of SEQ ID NO: 3 can be at the N-terminus or C-terminus of an amino acid sequence of SEQ ID NO: 51.

In some specific embodiments, the third domain (ABD) comprises or consists of an amino acid sequence of SEQ ID NO: 4. In some embodiments, the third domain comprises an amino acid sequence having at least 80%, 85%, 90%, 91%, 92%, 93%, 94%, 95%, 96%, 97%, 98%, 99% identify to SEQ ID NO: 4, and capable of binding to the angiopoietin (e.g., angiopoietin 2 or Ang2).

In some embodiments, the third domain (ABD) comprises or consists of an amino acid sequence of SEQ ID NO: 52. In some embodiments, the third domain comprises an amino acid sequence having at least 80%, 85%, 90%, 91%, 92%, 93%, 94%, 95%, 96%, 97%, 98%, 99% identify to SEQ ID NO: 52, and capable of binding to the angiopoietin (e.g., angiopoietin 2 or Ang2).

In some embodiments, the third domain (ABD) comprises or consists of an amino acid sequence of SEQ ID NO: 53. In some embodiments, the third domain comprises an amino acid sequence having at least 80%, 85%, 90%, 91%, 92%, 93%, 94%, 95%, 96%, 97%, 98%, 99% identify to SEQ ID NO: 53, and capable of binding to the angiopoietin (e.g., angiopoietin 2 or Ang2).

In some embodiments, the third domain (ABD) comprises or consists of an amino acid sequence of SEQ ID NO: 54. In some embodiments, the third domain comprises an amino acid sequence having at least 80%, 85%, 90%, 91%, 92%, 93%, 94%, 95%, 96%, 97%, 98%, 99% identify to SEQ ID NO: 54, and capable of binding to the angiopoietin (e.g., angiopoietin 2 or Ang2).

In certain embodiments, a third domain that binds to an angiopoietin (e.g., angiopoietin 1 and angiopoietin 2) is at N-terminus of the first domain or the second domain. In a specific embodiment, the polypeptide comprises, from N-terminus to C-terminus, the third domain, the first domain and the second domain. In another specific embodiment, the polypeptide comprises, from N-terminus to C-terminus, the third domain, the second domain, and the first domain.

In case wherein a fourth domain, i.e., a VEGFC binding domain (also referred to as Trap C) is in the fusion protein, in some embodiments, the VEGFC binding domain is derived from VEGFR-2 (e.g., domain 2). In other embodiments, the VEGFC binding domain is derived from VEGFR-3 (e.g., domain 1 and/or domain 2).

In some embodiments, the VEGFC binding domain comprises an amino acid sequence having at least 80%, 85%, 90%, 91%, 92%, 93%, 94%, 95%, 96%, 97%, 98%, 99% or 100% identify to SEQ ID NO: 55.

In some embodiments, the VEGFC binding domain comprises an amino acid sequence having at least 80%, 85%, 90%, 91%, 92%, 93%, 94%, 95%, 96%, 97%, 98%, 99% or 100% identify to SEQ ID NO: 56.

In some embodiments, the VEGFC binding domain comprises an amino acid sequence having at least 80%, 85%, 90%, 91%, 92%, 93%, 94%, 95%, 96%, 97%, 98%, 99% or 100% identify to SEQ ID NO: 57.

In some embodiments, the polypeptide further comprises an Fc region of an antibody. In some embodiment, the Fc region is derived from a human IgG. In some embodiments, the Fc region comprises an amino acid sequence of SEQ ID NO: 5. In some embodiments, the Fc region is at the C-terminus of the polypeptide. In other embodiments, the Fc region is not at the C-terminus of the polypeptide.

The various domains in the present fusion protein may present in any order. In some embodiments, the D2, D3, ABD, Fc region, and/or Trap C are in the order as shown in FIG. 1, FIG. 2, FIG. 8 and FIGS. 9A-9I. In some more specific embodiments, the D2, D3, ABD, Fc region, and/or Trap C are in the order as shown in FIG. 9A. In some more specific embodiments, the D2, D3, ABD, Fc region, and/or Trap C are in the order as shown in FIG. 9B. In some more specific embodiments, the D2, D3, ABD, Fc region, and/or Trap C are in the order as shown in FIG. 9C. In some more specific embodiments, the D2, D3, ABD, Fc region, and/or Trap C are in the order as shown in FIG. 9D. In some more specific embodiments, the D2, D3, ABD, Fc region, and/or Trap C are in the order as shown in FIG. 9E. In some more specific embodiments, the D2, D3, ABD, Fc region, and/or Trap C are in the order as shown in FIG. 9F. In some more specific embodiments, the D2, D3, ABD, Fc region, and/or Trap C are in the order as shown in FIG. 9G. In some more specific embodiments, the D2, D3, ABD, Fc region, and/or Trap C are in the order as shown in FIG. 9H. In some more specific embodiments, the D2, D3, ABD, Fc region, and/or Trap C are in the order as shown in FIG. 9I.

In some embodiments, the polypeptide further comprises a signal peptide at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the fusion proteins described herein will be evident to one of skill in the art. In some specific embodiments, the signal peptide comprises an amino acid sequence of SEQ ID NO: 6.

In some embodiments, the polypeptide further comprises one or more linkers between the above described various domains. The various domains described herein may be fused to each other via peptide linkers. In some embodiments, certain domains are directly fused to each other without any peptide linkers. The peptide linkers connecting different domains may be the same or different. In some embodiments, a polypeptide provided herein comprises a peptide linker between certain domains, but not other domains therein.

Each peptide linker in a polypeptide provided herein may have the same or different length and/or sequence. Each peptide linker may be selected and optimized independently. The length, the degree of flexibility and/or other properties of the peptide linker(s) used in the present fusion proteins may have some influence on properties, including but not limited to the affinity, specificity or avidity for one or more particular target molecules. In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, or about 30 amino acids to about 50 amino acids.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. In some embodiments, a peptide linker provided herein is a (GxS)n linker, wherein x and n, independently can be an integer between 3 and 12, including 3, 4, 5, 6, 7, 8, 9, 10, 11, 12, or more. Exemplary flexible linkers include but not limited to glycine polymers (G)n, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. Exemplary peptide linkers are listed in the table below.

TABLE 1
Exemplary Peptide Linkers

| Sequences | SEQ ID NO |
|---|---|
| DGGGS | SEQ ID NO: 24 |
| TGEKP | SEQ ID NO: 25 |
| GGRR | SEQ ID NO: 26 |
| GGGGSGGGGSGGGGGGSGSGGGGS | SEQ ID NO: 27 |
| GGGGSGGGGSGGGGGGSGSGGGGSGGGGSGGGGS | SEQ ID NO: 28 |
| EGKSSGSGSESKVD | SEQ ID NO: 29 |
| KESGSVSSEQLAQFRS | SEQ ID NO: 30 |
| GGRRGGGS | SEQ ID NO: 31 |
| LRQRDGERP | SEQ ID NO: 32 |
| LRQKDGGGSERP | SEQ ID NO: 33 |
| LRQKDGGGSGGGSERP | SEQ ID NO: 34 |
| GSTSGSGKPGSGEGST | SEQ ID NO: 35 |
| GSTSGSGKSSEGKG | SEQ ID NO: 36 |
| KESGSVSSEQLAQFRSLD | SEQ ID NO: 37 |
| (GS)ₙ, n is an integer including, e.g., 1, 2, 3, 4, 5, and 6. | SEQ ID NO: 38 |
| (GSGGS)ₙ, n is an integer including, e.g., 1, 2, 3, 4, 5, and 6. | SEQ ID NO: 39 |
| (GGGS)ₙ, n is an integer including, e.g., 1, 2, 3, 4, 5, and 6. | SEQ ID NO: 40 |
| (GGGGS)ₙ, n is an integer including, e.g., 1, 2, 3, 4, 5, and 6. | SEQ ID NO: 41 |
| (GGGGGS)ₙ, n is an integer including, e.g., 1, 2, 3, 4, 5, and 6. | SEQ ID NO: 42 |

The fusion protein of the present disclosure may comprise a hinge domain that is located between domains described above. A hinge domain is an amino acid segment that is generally found between two domains of a protein and may allow for flexibility of the protein and movement of one or both of the domains relative to one another.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. In some embodiments, the hinge domain is at least a portion of a hinge domain of a naturally occurring protein. Hinge domains of antibodies, such as an IgG, IgA, IgM, IgE, or IgD antibodies, are also compatible for use in the fusion proteins described herein. In some embodiments, the hinge domain is the hinge domain that joins the constant domains CHI and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the fusion protein described herein. In some embodiments, the linkers are self-cleavable linkers.

Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82: 1191-1197 (1990), and Bird et al., Science 242:423-426 (1988) may also be included in the fusion proteins provided herein, the disclosure of each of which is incorporated herein by reference.

In certain embodiments, the polypeptides provided herein are illustrated in FIG. 1.

In some specific embodiments, provided herein is a polypeptide comprising an amino acid sequence of SEQ ID NO: 7.

In some specific embodiments, provided herein is a polypeptide comprising an amino acid sequence of SEQ ID NO: 8.

In some specific embodiments, provided herein is a polypeptide comprising an amino acid sequence of SEQ ID NO: 9.

In some specific embodiments, provided herein is a polypeptide comprising an amino acid sequence of SEQ ID NO: 10.

In certain embodiments, the polypeptides provided herein are illustrated in FIG. 8.

In some specific embodiments, provided herein is a polypeptide comprising an amino acid sequence of SEQ ID NO: 58.

In some specific embodiments, provided herein is a polypeptide comprising an amino acid sequence of SEQ ID NO: 59.

In some specific embodiments, provided herein is a polypeptide comprising an amino acid sequence of SEQ ID NO: 60.

In some specific embodiments, provided herein is a polypeptide comprising an amino acid sequence of SEQ ID NO: 61.

In some specific embodiments, provided herein is a polypeptide comprising an amino acid sequence of SEQ ID NO: 62.

In some specific embodiments, provided herein is a polypeptide comprising an amino acid sequence of SEQ ID NO: 63.

In some specific embodiments, provided herein is a polypeptide comprising an amino acid sequence of SEQ ID NO: 64.

In some specific embodiments, provided herein is a polypeptide comprising an amino acid sequence of SEQ ID NO: 65.

In some specific embodiments, provided herein is a polypeptide comprising an amino acid sequence of SEQ ID NO: 66.

In certain embodiments, the fusion proteins described herein comprises amino acid sequences with certain percent identity relative to any one of polypeptides described in Section 6 below.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993) Such an algorithm is incorporated into the NBLAST and XBLAST programs of Altschul et al., J. Mol. Biol. 215:403 (1990). BLAST nucleotide searches can be performed with the NBLAST nucleotide program parameters sct. e.g., for score=100, word length=12 to obtain nucleotide sequences homologous to a nucleic acid molecules described herein. BLAST protein searches can be performed with the XBLAST program parameters set, e.g., to score 50, word length-3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In some embodiments, there is provided a polypeptide having at least about any one of 80%, 85%, 86%, 87%, 88%, 89%, 90%, 91%, 92%, 93%, 94%, 95%, 96%, 97%, 98%, 99%, or 100% sequence identity to an amino acid sequence selected from SEQ ID NOs: 7-10 and 58-66. In some embodiments, a polypeptide sequence having at least about any one of 80%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence, but the two domains or three domains within the polypeptide comprising that sequence retains the ability to bind to VEGF and one domain within the polypeptide retains the ability to bind to an angiopoietin (e.g., angiopoietin 1 and angiopoietin 2).

In some embodiments, a total of 1 to 10 amino acids have been substituted, inserted and/or deleted in an amino acid sequence selected from SEQ ID NOs: 7-10 and 58-66.

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 7, wherein the first domain and the second domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 8, wherein the first domain and the second domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 9, wherein the first domain and the second domain retain the ability to bind to VEGF and the third domain retains the ability to bind to angiopoietin.

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 10, wherein the first domain and the second domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 58, wherein the first domain, the second domain, and the fourth domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 59, wherein the first domain, the second domain, and the fourth domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 60, wherein the first domain, the second domain, and the fourth domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 61, wherein the first domain, the second domain, and the fourth domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 62, wherein the first domain, the second domain, and the fourth domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 63, wherein the first domain, the second domain, and the fourth domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 64, wherein the first domain, the second domain, and the fourth domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 65, wherein the first domain, the second domain, and the fourth domain retain the ability to bind to VEGF and the third domain retains the ability to bind to an angiopoietin (e.g., angiopoietin 1 and angiopoietin 2).

In certain embodiments, the fusion protein described herein comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 66, wherein the first domain, the second domain, and the fourth domain retain the ability to bind to VEGF and the third domain retains the ability to bind to an angiopoietin (e.g., angiopoietin 1 and angiopoietin 2).

Other exemplary fusion proteins provided herein are described in more detail in the following sections. In some embodiments, the fusion proteins according to any of the above embodiments may incorporate any of the features, singly or in combination, as described in Sections 5.2.2 to 5.2.4 below.

### 5.2.2. Variants and Modifications of Polypeptides

In some embodiments, amino acid sequence modification(s) of the fusion proteins described herein are contemplated. For example, it may be desirable to optimize the binding affinity and/or other biological properties of the fusion protein, including but not limited to specificity, thermostability, expression level, glycosylation, reduced immunogenicity, or solubility. Thus, in addition to the fusion protein described herein, it is contemplated that variants of the fusion protein described herein can be prepared. For example, peptide variants can be prepared by introducing appropriate nucleotide changes into the encoding DNA, and/or by synthesis of the desired polypeptide. Those skilled in the art who appreciate that amino acid changes may alter post-translational processes of the peptide.

Variations may be a substitution, deletion, or insertion of one or more codons encoding the polypeptide that results in a change in the amino acid sequence as compared with the original polypeptide.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental peptides.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include a polypeptide with an N-terminal methionyl residue.

Fusion proteins generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties. Exemplary substitutions are shown in the table below.

TABLE 2
Amino Acid Substitutions

| Original | Exemplary |
|---|---|
| Residue | Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the polypeptide provided herein also may be substituted, for example, with another amino acid, such as alanine or serine, to improve the oxidative stability of the molecule and to prevent aberrant crosslinking.

Non-conservative substitutions will entail exchanging a member of one of these classes for another class.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include a polypeptide with an N-terminal methionyl residue.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the polypeptide variant DNA.

Covalent modifications of the fusion proteins provided herein are included within the scope of the present disclosure. Covalent modifications include reacting targeted amino acid residues of a polypeptide provided herein with an organic derivatizing agent that is capable of reacting with selected side chains or the N- or C-terminal residues of the polypeptide. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

In some embodiments, the fusion proteins provided herein are chemically modified, for example, by the covalent attachment of any type of molecule to the fusion protein. The polypeptide derivatives may include polypeptide that have been chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the polypeptide may contain one or more non-classical amino acids.

In some embodiments, the fusion protein provided herein is altered to increase or decrease the extent to which the fusion protein is glycosylated. Addition or deletion of glycosylation sites to a polypeptide may be conveniently accomplished by altering the amino acid sequence such that one or more glycosylation sites is created or removed.

When the fusion provided herein is fused to an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the binding molecules provided herein may be made in order to create variants with certain improved properties.

In molecules that comprise a Fc region, one or more amino acid modifications may be introduced into the Fc region, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the present application contemplates variants that possesses some but not all effector functions, which make it a desirable candidate for applications in which the half life of the binding molecule in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the binding molecule lacks FcγR binding (hence likely lacking ADCC activity), but retains FcRn binding ability. Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96R non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the binding molecule is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12): 1759-1769 (2006)).

Binding molecules with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, a variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Binding molecules with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those molecules comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In some embodiments, it may be desirable to create cysteine engineered polypeptides, in which one or more residues of a polypeptide are substituted with cysteine residues. In some embodiments, the substituted residues occur at accessible sites of the peptide. By substituting those residues with cysteine, reactive thiol groups are thereby positioned at accessible sites of the peptide and may be used to conjugate the peptide to other moieties, such as drug moieties or linker-drug moieties, to create an immunoconjugate, as described further herein.

### 5.2.3. Preparation of Fusion Proteins

Also provided herein are methods for making the various fusion proteins provided herein.

In a specific embodiment, the fusion protein provided herein is recombinantly expressed. Recombinant expression of a fusion protein provided herein may require construction of an expression vector containing a polynucleotide that encodes the protein or a fragment thereof. Once a polynucleotide encoding a protein provided herein or a fragment thereof has been obtained, the vector for the production of the molecule may be produced by recombinant DNA technology using techniques well-known in the art. Thus, methods for preparing a protein by expressing a polynucleotide containing an encoding nucleotide sequence are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding a fusion protein provided herein, or a fragment thereof, operably linked to a promoter.

The expression vector can be transferred to a host cell by conventional techniques and the transfected cells are then cultured by conventional techniques to produce a fusion protein provided herein. Thus, also provided herein are host cells containing a polynucleotide encoding a fusion protein provided herein or fragments thereof operably linked to a heterologous promoter.

A variety of host-expression vector systems may be utilized to express the fusion protein provided herein. Such host-expression systems represent vehicles by which the coding sequences of interest may be produced and subsequently purified, but also represent cells which may, when transformed or transfected with the appropriate nucleotide coding sequences, express a fusion protein provided herein in situ. These include but are not limited to microorganisms such as bacteria (e.g., E. coli and B. subtilis) transformed with recombinant bacteriophage DNA, plasmid DNA or cosmid DNA expression vectors containing coding sequences; yeast (e.g., Saccharomyces Pichia) transformed with recombinant yeast expression vectors containing coding sequences; insect cell systems infected with recombinant virus expression vectors (e.g., baculovirus) containing coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or, eukaryotic cells, especially for the expression of whole recombinant molecule, can be used for the expression of a recombinant fusion protein. For example, mammalian cells such as Chinese hamster ovary cells (CHO), in conjunction with a vector such as the major intermediate early gene promoter element from human cytomegalovirus is an effective expression system for antibodies or variants thereof. In a specific embodiment, the expression of nucleotide sequences encoding the fusion proteins provided herein is regulated by a constitutive promoter, inducible promoter or tissue specific promoter.

In bacterial systems, a number of expression vectors may be advantageously selected depending upon the use intended for the fusion protein being expressed. For example, when a large quantity of such a fusion protein is to be produced, for the generation of pharmaceutical compositions of a fusion protein, vectors which direct the expression of high levels of fusion protein products that are readily purified may be desirable. Such vectors include, but are not limited to, the E. coli expression vector pUR278 (Ruther et al., EMBO 12:1791 (1983)), in which the coding sequence may be ligated individually into the vector in frame with the lac 7. coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the fusion protein in infected hosts (e.g., see Logan & Shenk, Proc. Natl. Acad. Sci. USA 8 1:355-359 (1984)). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7030 and HsS78Bst cells.

For long-term, high-yield production of recombinant proteins, stable expression can be utilized. For example, cell lines which stably express the fusion proteins may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the fusion protein. Such engineered cell lines may be particularly useful in screening and evaluation of compositions that interact directly or indirectly with the binding molecule.

A number of selection systems may be used, including but not limited to, the herpes simplex virus thymidine kinase (Wigler et al., Cell 11:223 (1977)), hypoxanthineguanine phosphoribosyltransferase (Szybalska & Szybalski, Proc. Natl. Acad. Sci. USA 48:202 (1992)), and adenine phosphoribosyltransferase (Lowy et al., Cell 22:8-17 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIB TECH 11(5):155-2 15 (1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds.), Current Protocols in Human Genetics, John Wiley & Sons, N Y (1994); Colberre-Garapin et al., J. Mol. Biol. 150:1 (1981), which are incorporated by reference herein in their entireties.

The expression level of a fusion protein can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing a fusion protein is amplifiable, increase in the level of inhibitor present in culture of host cell will increase the number of copies of the marker gene. Since the amplified region is associated with the fusion protein gene, production of the fusion protein will also increase (Crouse et al., Mol. Cell. Biol. 3:257 (1983)).

The host cell may be co-transfected with multiple expression vectors provided herein. The vectors may contain identical selectable markers which enable equal expression of respective encoding polypeptides. Alternatively, a single vector may be used which encodes, and is capable of expressing multiple polypeptides. The coding sequences may comprise cDNA or genomic DNA.

Once a fusion protein provided herein has been produced by recombinant expression, it may be purified by any method known in the art for purification of a polypeptide (e.g., an immunoglobulin molecule), for example, by chromatography (e.g., ion exchange, affinity, particularly by affinity for the specific antigen after Protein A, sizing column chromatography, and Kappa select affinity chromatography), centrifugation, differential solubility, or by any other standard technique for the purification of proteins. Further, the fusion protein molecules provided herein can be fused to heterologous polypeptide sequences described herein or otherwise known in the art to facilitate purification.

Various aspects of recombinant production of a fusion protein provided herein are described in more detail below in the context of prokaryotic cells or eukaryotic cells

### Recombinant Production in Prokaryotic Cells

Polynucleic acid sequences encoding the fusion protein of the present disclosure can be obtained using standard recombinant techniques. For example, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to, an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector of the present application may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the present fusion protein by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the -galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target peptide (Siebenlist et al. Cell 20: 269 (1980)) using linkers or adaptors to supply any required restriction sites.

In one aspect, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In some embodiments of the present disclosure, the signal sequences used in both cistrons of the expression system are STII signal sequences or variants thereof.

In some embodiments, the production of the fusion proteins according to the present disclosure can occur in the cytoplasm of the host cell, and therefore does not require the presence of secretion signal sequences within each cistron. Certain host strains (e.g., the E. coli trxB⁻ strains) provide cytoplasm conditions that are favorable for disulfide bond formation, thereby permitting proper folding and assembly of expressed protein subunits.

Prokaryotic host cells suitable for expressing the fusion proteins of the present disclosure include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In one embodiment, E. coli cells are used as hosts. Examples of E. coli strains include strain W3110 (Bachmann, Cellular and Molecular Biology, vol 2 (Washington, D.C. American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon.

Typically the host cell should secrete minimal amounts of proteolytic enzymes, and additional protease inhibitors may desirably be incorporated in the cell culture.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the fusion proteins of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39ºC, more preferably from about 25° ° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods 263:133-147 (2002)). A variety of other inducers may be used, according to the vector construct employed, as is known in the art.

The expressed fusion proteins of the present disclosure are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

During the fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the fusion proteins of the present disclosure, various fermentation conditions can be modified. For example, to improve the proper assembly and folding of the secreted polypeptides, additional vectors overexpressing chaperone proteins, such as Dsb proteins (DsbA, DsbB, DsbC, DsbD and or DsbG) or FkpA (a peptidylprolyl cis, trans-isomerase with chaperone activity) can be used to co-transform the host prokaryotic cells. The chaperone proteins have been demonstrated to facilitate the proper folding and solubility of heterologous proteins produced in bacterial host cells. Chen et al. J Bio Chem 274:19601-19605 (1999); U.S. Pat. Nos. 6,083,715; 6,027,888; Bothmann and Pluckthun, J. Biol. Chem. 275:17100-17105 (2000); Ramm and Pluckthun, J. Biol. Chem. 275:17106-17113 (2000); Arie et al., Mol. Microbiol. 39:199-210 (2001).

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, U.S. Pat. Nos. 5,264,365; 5,508,192; Hara et al., Microbial Drug Resistance, 2:63-72 (1996).

E. coli strains deficient for proteolytic enzymes and transformed with plasmids overexpressing one or more chaperone proteins may be used as host cells in the expression system encoding the antibodies of the present application.

The fusion proteins produced herein can be further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAF, chromatofocusing, SDS-PAGE, ammonium sulfate precipitation, and gel filtration using, for example, Sephadex G-75.

Protein A immobilized on a solid phase for example can be used in some embodiments for immunoaffinity purification of binding molecules of the present disclosure. The solid phase to which Protein A is immobilized is preferably a column comprising a glass or silica surface, more preferably a controlled pore glass column or a silicic acid column. In some embodiments, the column has been coated with a reagent, such as glycerol, in an attempt to prevent nonspecific adherence of contaminants. The solid phase is then washed to remove contaminants non-specifically bound to the solid phase. Finally the antibodies of interest is recovered from the solid phase by elution.

### Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region can be ligated in reading frame to DNA encoding the antibodies of the present application.

Generally, the origin of replication component is not needed for mammalian expression vectors (the SV40 origin may typically be used only because it contains the early promoter).

Expression and cloning vectors may contain a selection gene, also termed a selectable marker. Selection genes may encode proteins that confer resistance to antibiotics or other toxins, e.g., ampicillin, neomycin, methotrexate, or tetracycline; complement auxotrophic deficiencies; or supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the antibodies of the present application. For example, cells transformed with the DHFR selection gene are first identified by culturing all of the transformants in a culture medium that contains methotrexate (Mtx), a competitive antagonist of DHFR. An exemplary appropriate host cell when wild-type DHFR is employed is the Chinese hamster ovary (CHO) cell line deficient in DHFR activity. Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic.

Expression and cloning vectors usually contain a promoter that is recognized by the host organism and is operably linked to the nucleic acid encoding the desired polypeptide sequences. Eukaryotic genes have an AT-rich region located approximately 25 to 30 based upstream from the site where transcription is initiated. Another sequence found 70 to 80 bases upstream from the start of the transcription of many genes may be included. The 3′ end of most eukaryotic may be the signal for addition of the poly A tail to the 3′ end of the coding sequence. All of these sequences may be inserted into eukaryotic expression vectors.

Polypeptide transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding the fusion proteins of the present disclosure by higher eukaryotes is often increased by inserting an enhancer sequence into the vector. Many enhancer sequences are now known from mammalian genes (globin, elastase, albumin, α-fetoprotein, and insulin). Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

Expression vectors used in eukaryotic host cells (yeast, fungi, insect, plant, animal, human, or nucleated cells from other multicellular organisms) also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region.

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CVI line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells can be transformed with the above-described expression or cloning vectors for protein production and cultured in conventional nutrient media modified as appropriate for inducing promoters, selecting transformants, or amplifying the genes encoding the desired sequences.

The host cells used to produce the fusion protein of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the fusion proteins can be produced intracellularly, in the periplasmic space, or directly secreted into the medium. If the fusion protein is produced intracellularly, as a first step, the particulate debris, either host cells or lysed fragments, are removed, for example, by centrifugation or ultrafiltration. Where the fusion protein is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The matrix to which the affinity ligand is attached is most often agarose, but other matrices are available. Mechanically stable matrices such as controlled pore glass or poly (styrene-divinyl) benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the polypeptide to be recovered. Following any preliminary purification step(s), the mixture comprising the polypeptide of interest and contaminants may be subjected to low pH hydrophobic interaction chromatography.

### 5.2.4. Other Binding Molecules Comprising the Fusion Proteins

In another aspect, provided herein is a binding molecule comprising a fusion protein provided herein. In some embodiments, a fusion protein provided herein is part of other binding molecules. Exemplary binding molecules of the present disclosure are described herein.

### Other Fusion Proteins

In various embodiments, the fusion protein provided herein can be genetically fused or chemically conjugated to another agent, for example, protein-based entities. The fusion protein may be chemically-conjugated to the agent, or otherwise non-covalently conjugated to the agent. The agent can be a peptide or antibody (or a fragment thereof).

Thus, in some embodiments, provided herein are fusion proteins as described above that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, about 100, about 150, about 200, about 250, about 300, about 350, about 400, about 450 or about 500 amino acids, or over 500 amino acids) to generate fusion proteins, as well as uses thereof.

Moreover, the fusion protein provided herein can be fused to marker or “tag” sequences, such as a peptide, to facilitate purification. In specific embodiments, the marker or tag amino acid sequence is a hexa-histidine peptide, hemagglutinin (“HA”) tag, and “FLAG” tag.

The fusion protein provided herein can be fused or conjugated to an antibody. Methods for fusing or conjugating moieties (including polypeptides) to an antibody are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., Immunol. Rev. 62:119-58 (1982); U.S. Pat. Nos. 5,336,603, 5,622,929; 5,359,046; 5,349,053, 5,447,851; 5,723, 125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-39 (1991); Traunecker et al., Nature, 331:84-86 (1988); Zheng et al., J. Immunol. 154:5590-600 (1995); and Vil et al., Proc. Natl. Acad. Sci. USA 89:11337-41 (1992)).

Fusion proteins may be generated, for example, through the techniques of gene-shuffling, motif-shuffling, exon-shuffling, and/or codon-shuffling (collectively referred to as “DNA shuffling”).

In various embodiments, the fusion protein is genetically fused to the agent. Genetic fusion may be accomplished by placing a linker (e.g., a polypeptide) between the fusion protein and the agent. The linker may be a flexible linker.

In various embodiments, the fusion protein is genetically conjugated to a therapeutic molecule, with a hinge region linking the fusion protein to the therapeutic molecule.

In some embodiments, the linkers are peptide linkers described in Section 5.2.1 above. These other fusion proteins can be made according the methods well known in the art as well as described in Section 5.2.3 above.

### Immunoconjugates

In some embodiments, the present disclosure also provides immunoconjugates comprising any of the fusion protein described herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

The drugs include but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45 4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In some embodiments, an immunoconjugate comprises the fusion protein as described herein conjugated to an enzymatically active toxin or fragment thereof, including but not limited to diphtheria A chain, nonbinding active fragments of diphtheria toxin. exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, an immunoconjugate comprises the fusion protein as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of a fusion protein and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the polypeptide. See WO94/11026.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

The immunuoconjugates provided herein contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### 5.3. Recombinant Viral Vectors and Viral Particles for Gene Therapies

Also provided herein are virus based gene therapies for delivering the fusion protein provided herein. Thus, in another aspect, provided herein are viral vectors (e.g., rAAV vectors) comprising a nucleic acid (as a transgene) encoding the fusion protein provided herein. In yet another aspect, provided herein are viral particles (e.g., rAAVs or rAAV particles) comprising a nucleic acid (as a transgene) encoding the fusion protein provided herein.

### 5.3.1. Virus Based Gene Delivery Systems

A number of viral based systems have been developed for gene transfer into mammalian cells. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

In certain embodiments, the viral vector or viral particle provided herein is derived from an adenovirus. Exemplary vectors are based on or derived from HAd5, ChAd3, HAd26, HAd6, AdCH3NSmut, HAd35, ChAd63, HAd4, rcAd26. Recombinant adenovirus vectors can be constructed according to known methods in the art. See, e.g., O'Connor et al., Virology, 217(1): 11-22 (1996); Hardy et al., Journal of Virology, 73(9): 7835-7841 (1999); Hardy et al., Journal of Virology, 71(3): 1842-1849 (1997). In some embodiments, third-generation adenoviral vectors (also called “high capacity adenoviral vectors” (HCAds), helper-dependent or “gutless” adenoviral vectors) can be used herein to deliver longer sequences. In some embodiments, the polynucleotide of interest, e.g., a transgene is cloned into an adenoviral vector that only contains the ITRs and a packaging signal. A helper adenoviral vector may be co-transfected into HEK cells to generate the adenoviral particle. See Lee et al., Genes and Diseases, 4(2): 43-63 (2007).

In certain embodiments, the viral vector or viral particle provided herein is derived from a lentivirus. Exemplary vectors are based on or derived from HIV-1, HIV-2, SIVSM, SIVAGM, EIAV, FIV, VNV, CAEV, or BIV. Lentiviral vectors can be produced according to the known methods in the art, e.g., as described in Cribbs et al., BMC Biotechnology, 13:98 (2003); Merten et al., Mol Ther Methods Clin Dev., 13 (3):16017 (2016); Durand and Cimarelli, Viruses, 3:132-159 (2011). In some embodiments, third-generation self-inactivating lentiviral vectors are used herein.

In certain embodiments, the viral vector or viral particle provided herein is derived from a herpes simplex virus (HSV). In some embodiments, the herpes simplex virus is a herpes simplex type 1 virus (HSV-1), a herpes simplex type 2 virus (HSV-2), or any derivative thereof. Exemplary vectors are based on or derived from HSV-1, HSV-2, CMV, VZV, EBV, and KSHV. HSV-based vectors can be constructed according the methods known in the art, e.g., as described in U.S. Pat. Nos. 7,078,029, 6,261,552, 5,998,174, 5,879,934, 5,849,572, 5,849,571, 5,837,532, 5,804,413, and 5,658,724, and International Patent Applications WO 91/02788, WO 96/04394, WO 98/15637, and WO 99/06583, which are incorporated herein by reference in their entireties.

In some embodiments, the HSV-based vector provided herein is an amplicon vector. In other embodiments, the HSV-based vector provided herein is a replication-defective vector. In yet other embodiments, the HSV-based vector provided herein is a replication-competent vector.

The amplicons are plasmid-derived vectors engineered to contain both the origin of HSV DNA replication (ori) and HSV cleavage-packaging recognition sequences (pac). When amplicons are transfected into mammalian cells with HSV helper functions, they are replicated, form head-to-tail linked concatamers and are then packaged into viral particles. There are two major methods currently used for producing amplicon particles, one based on infection with defective helper HSVs and the other based on transfection of HSV-1 genes, such as a set of pac-deleted overlapping cosmids or a pac-deleted and ICP27-deleted BAC-HSV-1. In some embodiments, amplicons used herein can accommodate large fragments of foreign DNA (e.g., up to 152 kb), including multiple copies of the transgene (e.g., up to 15 copies), and are non-toxic.

In some embodiments, an HSV-based vector used herein is deficient in at least one essential HSV gene, and the HSV-based vector may also comprise one or more deletions of non-essential genes. In some embodiments, the HSV-based vector is replication-deficient. Most replication-deficient HSV-based vectors contain a deletion to remove one or more intermediate-early, early, or late HSV genes to prevent replication. In other embodiments, the HSV-based vector is deficient in an immediate early gene selected from the group consisting of ICP0, ICP4, ICP22, ICP27, ICP47, and a combination thereof. In a specific embodiment, the HSV-based vector is deficient for all of ICP0, ICP4, ICP22, ICP27, and ICP47. Exemplary replication-competent vectors include NV-1020 (HSV-1), RAV9395 (HSV-2), AD-472 (HSV-2), NS-gEnull (HSV-1), and ImmunoVEX (HSV2). Exemplary replication-defective vectors include dl5-29 (HSV-2), d15-29-41L (HSV-1), DISC-dH (HSV-1 and HSV-2), CJ9gD(HSV-1), TOH-OVA (HSV-1), d106 (HSV-1), d81(HSV-1), HSV-SIV d106(HSV-1), and d106 (HSV-1).

Replication-deficient HSV-based vectors are typically produced in complementing cell lines that provide gene functions not present in the replication-deficient HSV-based vectors, but required for viral propagation, at appropriate levels in order to generate high titers of viral vector stock. An exemplary cell line complements for at least one and, in some embodiments, all replication-essential gene functions not present in a replication-deficient HSV-based vector. For example, a HSV-based vector deficient in ICP0, ICP4, ICP22, ICP27, and ICP47 can be complemented by the human osteosarcoma line U2OS. The cell line can also complement non-essential genes that, when missing, reduce growth or replication efficiency (e.g., UL55). The complementing cell line can complement for a deficiency in at least one replication-essential gene function encoded by the early regions, immediate-early regions, late regions, viral packaging regions, virus-associated regions, or combinations thereof, including all HSV functions (e.g., to enable propagation of HSV amplicons, which comprise minimal HSV sequences, such as only inverted terminal repeats and the packaging signal or only ITRs and an HSV promoter). In some embodiments, the cell line is further characterized in that it contains the complementing genes in a non-overlapping fashion with the HSV-based vector, which minimizes, and practically eliminates, the possibility of the HSV-based vector genome recombining with the cellular DNA. Accordingly, the presence of replication competent HSV is minimized, if not avoided in the vector stock, which, therefore, is suitable for certain therapeutic purposes, especially gene therapy purposes. The construction of complementing cell lines involves standard molecular biology and cell culture techniques well known in the art.

In certain embodiments, the viral vector or viral particle provided herein is derived from an adeno-associated virus (AAV). More detailed description related to AAV is provided in Sections 5.3.2-5.3.4 below.

The nucleic acid of interest can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present disclosure. Promoters may be roughly categorized as constitutive promoters or regulated promoters, such as inducible promoters.

In some embodiments, the nucleic acid encoding the fusion protein is operably linked to a constitutive promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1 alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies.

In some embodiments, the nucleic acid encoding the fusion protein is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof.

In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In some embodiments, the vector also contains a selectable marker gene or a reporter gene to select cells expressing the fusion protein from the population of host cells transfected through vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

### 5.3.2. Recombinant AAV Vectors

In certain more specific embodiments, the fusion protein provided herein is delivered by a AAV based system, and thus is included in a recombinant AAV vector.

Any AAV serotypes or variants thereof can be used in the present disclosure. AAV serotypes may include, but not limited to, AAV1 (Genbank Accession No. NC_002077.1; HC000057.1), AAV2 (Genbank Accession No. NC_001401.2, JC527779.1), AAV2i8 (Asokan, A., 2010, Discov. Med. 9:399), AAV3 (Genbank Accession No. NC_001729.1), AAV3-B (Genbank Accession No. AF028705.1), AAV4 (Genbank Accession No. NC_001829.1), AAV5 (Genbank Accession No. NC_006152.1; JC527780.1), AAV6 (Genbank Accession No. AF028704.1; JC527781.1), AAV7 (Genbank Accession No. NC_006260.1; JC527782.1), AAV8 (Genbank Accession No. NC_006261.1; JC527783.1), AAV9 (Genbank Accession No AX753250.1; JC527784.1), AAV10 (Genbank Accession No AY631965.1), AAVrh10 (Genbank Accession No. AY243015.1), AAV11 (Genbank Accession No AY631966.1), AAV12 (Genbank Accession No DQ813647.1), AAV13 (Genbank Accession No EU285562.1), AAV LK03, AAVrh74, AAV DJ (Wu Z, et al., J Virol. 80:11393-7 (2006)), AAVAnc81, Anc82, Anc83, Anc84, Anc110, Anc113, Anc126, or Anc127 (Zin, E. et al., Cell. Rep. 12:1056 (2016)), AAV_go.1 (Arbetum, A. E. et al., J. Virol. 79:15238 (2005)), AAVhu.37, AAVrh8, AAVrh8R, and AAV rh.8 (Wang et al., Mol. Ther. 18:119-125 (2010), or variants thereof.

AAV variants include, but not limited to, AAV1 variants (e.g., AAV comprising AAV1 variant capsid proteins), AAV2 variants (e.g., AAV comprising AAV2 variant capsid proteins), AAV3 variants (e.g., AAV comprising AAV3 variant capsid proteins), AAV3-B variants (e.g., AAV comprising AAV3-B variant capsid proteins), AAV4 variants (e.g., AAV comprising AAV4 variant capsid proteins), AAV5 variants (e.g., AAV comprising AAV5 variant capsid proteins), AAV6 variants (e.g., AAV comprising AAV6 variant capsid proteins), AAV7 variants (e.g., AAV comprising AAV7 variant capsid proteins), AAV8 variants (e.g., AAV comprising AAV8 variant capsid proteins), AAVrh8, AAVrh8R (e.g., AAV comprising AAVrh8 or AAVrh8R variant capsid proteins), AAV9 variants (e.g., AAV comprising AAV9 variant capsid proteins), AAV10 variants (e.g., AAV comprising AAV10 variant capsid proteins), AAVrh10 variants (e.g., AAV comprising AAVrh10 variant capsid proteins), AAV11 variants (e.g., AAV comprising AAV11 variant capsid proteins), AAV12 variants (e.g., AAV comprising AAV12 variant capsid proteins), AAV13 variants (e.g., AAV comprising AAV13 variant capsid proteins), AAV LK03 variants (e.g., AAV comprising AAV LK03 variant capsid proteins), or AAVrh74 variants (e.g., AAV comprising AAVrh74 variant capsid proteins).

Recombinant AAV (rAAV) vectors used in the present disclosure can be constructed according to known techniques. In some embodiments, the rAAV vector is constructed to include operatively linked components in the direction of transcription, control elements including a transcriptional initiation region, the polynucleotide encoding the fusion protein provided herein and a transcriptional termination region. The control elements can be selected based on the cell of interest. In some embodiments, the resulting rAAV vector construct comprising the operatively linked components is franked (5′ and 3′) with functional AAV ITR sequences.

In certain embodiments, the polypeptide encoding the fusion protein is operatively linked to at least one regulatory sequence. In certain embodiments, regulatory sequences may, for example, include promoter sequences, enhancer sequences, e.g., upstream enhancer sequences (USEs), RNA processing signals, e.g., splicing signals, polyadenylation signal sequences, sequences that stabilize cytoplasmic mRNA, post-transcriptional regulatory elements (PREs) and/or microRNA (miRNA) target sequences. In certain embodiments, regulatory sequences may include sequences that enhance translation efficiency (e.g., Kozak sequences), sequences that enhance protein stability, and/or sequences that enhance protein processing and/or secretion. In certain embodiments, the polynucleotide may encode regulatory miRNAs.

In certain embodiments, a regulatory sequence comprises a constitutive promoter and/or regulatory control element. In certain embodiments, a regulatory sequence comprises a regulatable promoter and/or regulatory control element. In certain embodiments, a regulatory sequence comprises a ubiquitous promoter and/or regulatory control element. In certain embodiments, a regulatory sequence comprises a cell- or tissue-specific promoter and/or regulatory control element. In certain embodiments, the regulatory control element is 5′ of the coding sequence of the fusion protein (that is, is present in ‘5 untranslated regions; 5’ UTRs). In other embodiments, the regulatory control element is 3′ of the coding sequence of the fusion protein (that is, is present in ‘3 untranslated regions; 3’ UTRs). In certain embodiments, the polynucleotide comprises more than one regulatory control element, for example may comprise two, three, four or five control elements. In instances wherein the polynucleotide comprises more than one control element, each control element may independently be 5′ of, 3′ of, flank, or within the coding sequence of the fusion protein.

In certain embodiments, the control element is an enhancer. In some embodiments, the control elements included direct the transcription or expression of the polynucleotide of the fusion protein in the subject in vivo. Control elements can comprise control sequences normally associated with the selected polynucleotide of interest or alternatively heterologous control sequences.

Exemplary control sequences include those derived from sequences encoding mammalian or viral genes, such as neuron-specific enolase promoter, a GFAP promoter, the SV40 early promoter, mouse mammary tumor virus LTR promoter, adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, and hybrid promoters.

In certain embodiments, a promoter is not cell- or tissue-specific., e.g., the promoter is considered a ubiquitous promoter. Examples of promoter sequences that may promote expression in multiple cell or tissue types include, for example, human elongation factor la-subunit (EFla), cytomegalovirus (CMV) immediate-early enhancer and/or promoter, chicken beta-actin (CBA) and its derivatives, e.g., CAG, for example, a CBA promoter with an S40 intron, beta glucuronidase (GUSB), or ubiquitin C (UBC).

In certain embodiments, a promoter sequence can promote expression in particular cell types or tissues. For example, in certain embodiments, a promoter may be a muscle-specific promoter, e.g., may be a mammalian muscle creatine kinase (MCK) promoter, mammalian desmin (DES) promoter, mammalian troponin I (TNNI2) promoter, or a mammalian skeletal alpha-actin (ASKA) promoter. In other embodiments, a promoter sequence may be able to promote expression in neural cells or cell types, e.g., may be a neuron-specific enolase (NSE), synapsin (Syn), methyl-CpG binding protein 2 (MeCP2), Ca2+/calmodulin-dependent protein kinase II (Ca.MKII), metabotropic glutamate receptor 2 (mGluR2), neurofilament light (NFL) or heavy (NFH), beta-globin minigene hb2, preproenkephalin (PPE), enkephalin (Enk) or excitatory amino acid transporter 2 (EAAT2) promoter. In other embodiments, a promoter sequence may promote expression in the liver, e.g., may be an alpha-1-antitrypsin (hAAT) or thyroxine binding globulin (TBG) promoter. In yet other embodiments, a promoter sequence may promote expression in cardiac tissue, e.g., may be a cardiomyocyte-specific promoter such as an MHC, cTnT, or CMV-MUC2k promoter.

In certain embodiments, the polynucleotide may comprise at least one polyadenylation (polyA) signal sequence, which are well known in the art. In instances where a polyadenylation sequence is present, it is generally located between the 3′ end of the transgene coding sequence and the 5′ end of the 3′ ITR. In certain embodiments, the polynucleotide further comprises a polyA upstream enhancer sequence 5′ of the polyA signal sequence. In certain instances, the regulatory sequence is a sequence that increases translation efficiency, e.g., a Kozak sequence.

In certain embodiments, the polynucleotide comprises an intron. In certain embodiments, the intron is present within the coding sequence of the fusion protein provided herein. In certain embodiments, the intron is 5′ or 3′ of the coding sequence of the fusion protein. In certain embodiments, the intron flanks the 5′ or 3′ terminus of the coding sequence of the fusion protein. In certain embodiments, the polynucleotide comprises two introns. In some embodiments, one intron is 5′ of and one intron is 3′ of the coding sequence of the fusion protein. In certain embodiments, one intron flanks the 5′ terminus of the coding sequence of the fusion protein and the second intron flanks the 3′ terminus of the coding sequence of the fusion protein. In certain embodiments, the intron is an SV40 intron, e.g., a 5′ UTR SV40 intron.

The sequences of AAV ITR known in the art can be used in the present rAAV vector In some embodiments, the AAV ITR used in the present vectors has a wild-type nucleotide sequence. In other embodiments, the AAV ITR sequence used in the present vectors is not wild-type sequence, and instead it comprises, e.g., the insertion, deletion or substitution of nucleotides. AAV ITRs provided herein may be derived from any AAV serotypes, including but not limited to, AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a variant thereof.

In some embodiments, the 5′ and 3′ ITRs which flank a nucleotide sequence in a rAAV vector provided herein are identical and derived from the same AAV serotype. In other embodiments, the 5′ and 3′ ITRs which flank a nucleotide sequence in a rAAV vector provided herein are different and/or derived from different AAV serotypes.

In some embodiments, the rAAV vector comprising the polynucleotide of the fusion protein flanked by AAV ITRs can be constructed by directly inserting the polynucleotide of interest into an AAV genome, e.g., into an excised AAV open reading frames, and certain portions of the AAV genome can optionally be deleted, as described in, e.g., WO 1993/003769; Kotin (1994) Human Gene Therapy 5:793-801; Shelling and Smith (1994) Gene Therapy 1.165-169, and Zhou et al. (1994) J. Exp. Med. 179: 1867-1875.

In other embodiments. AAV ITRs are excised from an AAV genome or from an AAV vector containing such ITRs, and then are fused to 5′ and 3′ of a polynucleotide sequence of the fusion protein that is present in another vector using standard ligation techniques.

In certain embodiments, the rAAV vector provided herein comprises a recombinant self-complementing genome. A rAAV comprising a self-complementing genome can usually quickly form a double stranded DNA molecule by its partially complementing sequences (e.g., complementing coding and non-coding strands of a transgene). More specifically, in some embodiments, an rAAV vector provided herein comprises an rAAV genome that comprises a first heterologous polynucleotide sequence (e.g., a therapeutic transgene coding strand) and a second heterologous polynucleotide sequence (e.g., the noncoding or antisense strand of the therapeutic transgene), and the first heterologous polynucleotide sequence can form intrastrand base pairs with the second polynucleotide sequence. In some embodiments, the first heterologous polynucleotide sequence and a second heterologous polynucleotide sequence are linked by a sequence that facilitates intrastrand base-pairing, e.g., a hairpin DNA structure. In some embodiments, the first heterologous polynucleotide sequence and a second heterologous polynucleotide sequence are linked by a mutated ITR, so that the rep proteins do not cleave the viral genome at the mutated ITR. rAAV vectors comprising self-complementing genomes can be made using the methods known in the art, e.g., as described in U.S. Pat. Nos. 7,125,717; 7,785,888; 7,790,154; 7,846,729; 8,093,054; and 8,361,457.

In some embodiments, the polynucleotide molecules in the rAAV vectors provided herein is less than about 5 kilobases (kb) in size. In some embodiments, the polynucleotide molecules in the rAAV vectors provided herein is less than about 4.5 kb in size. In some embodiments, the polynucleotide molecules in the rAAV vectors provided herein is less than about 4.0 kb in size. In some embodiments, the polynucleotide molecules in the rAAV vectors provided herein is less than about 3.5 kb in size. In some embodiments, the polynucleotide molecules in the rAAV vectors provided herein is less than about 3.0 kb in size. In some embodiments, the polynucleotide molecules in the rAAV vectors provided herein is less than about 2.5 kb in size.

In certain embodiments, provided herein is a recombinant AAV (rAAV) vector comprising a nucleic acid encoding a polypeptide wherein the polypeptide comprises (i) a first domain derived from VEGFR-1; (ii) a second domain derived from VEGFR-2; and (iii) a third domain that is capable of binding to an angiopoietin (e.g., angiopoietin 1 and angiopoietin 2), wherein the rAAV vector comprises an inverted terminal repeat (ITR) from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a combination or variant thereof. In some embodiments, the third domain is at N-terminus of the first domain and the second domain.

In certain embodiments, provided herein is a recombinant AAV (rAAV) vector comprising a nucleic acid encoding a polypeptide wherein the polypeptide comprises (i) a first domain derived from VEGFR-1; (ii) a second domain derived from VEGFR-2; (iii) a third domain that is capable of binding to an angiopoietin (e.g., angiopoietin 1 and angiopoietin 2), and (iv) a fourth domain that is capable of binding to VEGFC, wherein the rAAV vector comprises an inverted terminal repeat (ITR) from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a combination or variant thereof. In some embodiments, the third domain is at N-terminus of the first domain and the second domain.

In some specific embodiments, the rAAV vector comprises a nucleic acid encoding a polypeptide having SEQ ID NO: 7 or a polypeptide having at least 80%, 85%, 90%, 91%, 92%, 93%, 94%, 95%, 96%, 97%, 98%, 99% sequence identify to SEQ ID NO: 7. In some embodiments, the rAAV vector comprises an ITR from AAV1. In some embodiments, the rAAV vector comprises an ITR from AAV2. In some embodiments, the rAAV vector comprises an ITR from AAV2i8 In some embodiments, the rAAV vector comprises an ITR from AAV3. In some embodiments, the rAAV vector comprises an ITR from AAV3-B. In some embodiments, the rAAV vector comprises an ITR from AAV4 In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12. In some embodiments, the rAAV vector comprises an ITR from AAV13 In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 8 or a polypeptide having at least 80%, 85%, 90%, 91%, 92%, 93%, 94%, 95%, 96%, 97%, 98%, 99% sequence identify to SEQ ID NO: 8. In some embodiments, the rAAV vector comprises an ITR from AAV1. In some embodiments, the rAAV vector comprises an ITR from AAV2. In some embodiments, the rAAV vector comprises an ITR from AAV2i8 In some embodiments, the rAAV vector comprises an ITR from AAV3. In some embodiments, the rAAV vector comprises an ITR from AAV3-B. In some embodiments, the rAAV vector comprises an ITR from AAV4 In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12 In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 9 or a polypeptide having at least 80%, 85%, 90%, 91%, 92%, 93%, 94%, 95%, 96%, 97%, 98%, 99% sequence identify to SEQ ID NO: 9. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4 In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12 In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 10 or a polypeptide having at least 80%, 85%, 90%, 91%, 92%, 93%, 94%, 95%, 96%, 97%, 98%, 99% sequence identify to SEQ ID NO: 10. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10 In some embodiments, the rAAV vector comprises an ITR from AAV11. In some embodiments, the rAAV vector comprises an ITR from AAV12. In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 58 or a polypeptide having at least 80%, 85%, 90%, 91%, 92%, 93%, 94%, 95%, 96%, 97%, 98%, 99% sequence identify to SEQ ID NO: 58. In some embodiments, the rAAV vector comprises an ITR from AAV1. In some embodiments, the rAAV vector comprises an ITR from AAV2 In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4 In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12 In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 59 or a polypeptide having at least 80%, 85%, 90%, 91%, 92%, 93%, 94%, 95%, 96%, 97%, 98%, 99% sequence identify to SEQ ID NO: 59. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12. In some embodiments, the rAAV vector comprises an ITR from AAV13 In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 60 or a polypeptide having at least 80%, 85%, 90%, 91%, 92%, 93%, 94%, 95%, 96%, 97%, 98%, 99% sequence identify to SEQ ID NO: 60. In some embodiments, the rAAV vector comprises an ITR from AAV1. In some embodiments, the rAAV vector comprises an ITR from AAV2. In some embodiments, the rAAV vector comprises an ITR from AAV2i8 In some embodiments, the rAAV vector comprises an ITR from AAV3. In some embodiments, the rAAV vector comprises an ITR from AAV3-B. In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12 In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 61 or a polypeptide having at least 80%, 85%, 90%, 91%, 92%, 93%, 94%, 95%, 96%, 97%, 98%, 99% sequence identify to SEQ ID NO: 61. In some embodiments, the rAAV vector comprises an ITR from AAV1. In some embodiments, the rAAV vector comprises an ITR from AAV2. In some embodiments, the rAAV vector comprises an ITR from AAV2i8 In some embodiments, the rAAV vector comprises an ITR from AAV3. In some embodiments, the rAAV vector comprises an ITR from AAV3-B. In some embodiments, the rAAV vector comprises an ITR from AAV4 In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12. In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 62 or a polypeptide having at least 80%, 85%, 90%, 91%, 92%, 93%, 94%, 95%, 96%, 97%, 98%, 99% sequence identify to SEQ ID NO: 62. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10 In some embodiments, the rAAV vector comprises an ITR from AAV11. In some embodiments, the rAAV vector comprises an ITR from AAV12 In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 63 or a polypeptide having at least 80%, 85%, 90%, 91%, 92%, 93%, 94%, 95%, 96%, 97%, 98%, 99% sequence identify to SEQ ID NO: 63. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10 In some embodiments, the rAAV vector comprises an ITR from AAV11. In some embodiments, the rAAV vector comprises an ITR from AAV12. In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 64 or a polypeptide having at least 80%, 85%, 90%, 91%, 92%, 93%, 94%, 95%, 96%, 97%, 98%, 99% sequence identify to SEQ ID NO: 64. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12. In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 65 or a polypeptide having at least 80%, 85%, 90%, 91%, 92%, 93%, 94%, 95%, 96%, 97%, 98%, 99% sequence identify to SEQ ID NO: 65. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11 In some embodiments, the rAAV vector comprises an ITR from AAV12. In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the rAAV vector comprises a nucleic acid encoding a polypeptide having SEQ ID NO: 66 or a polypeptide having at least 80%, 85%, 90%, 91%, 92%, 93%, 94%, 95%, 96%, 97%, 98%, 99% sequence identify to SEQ ID NO 66. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV 10. In some embodiments, the rAAV vector comprises an ITR from AAVrh10 In some embodiments, the rAAV vector comprises an ITR from AAV11. In some embodiments, the rAAV vector comprises an ITR from AAV12. In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some more specific embodiments, provided herein is a vector comprising a nucleic acid sequence of SEQ ID NO: 11, or a nucleic acid sequence having at least 80%, 85%, 90%, 91%, 92%, 93%, 94%, 95%, 96%, 97%, 98%, 99% identify to SEQ ID NO: 11.

In some more specific embodiments, provided herein is a vector comprising a nucleic acid sequence of SEQ ID NO: 12, or a nucleic acid sequence having at least 80%, 85%, 90%, 91%, 92%, 93%, 94%, 95%, 96%, 97%, 98%, 99% identify to SEQ ID NO: 12.

In some more specific embodiments, provided herein is a vector comprising a nucleic acid sequence of SEQ ID NO: 13, or a nucleic acid sequence having at least 80%, 85%, 90%, 91%, 92%, 93%, 94%, 95%, 96%, 97%, 98%, 99% identify to SEQ ID NO: 13.

In some more specific embodiments, provided herein is a vector comprising a nucleic acid sequence of SEQ ID NO: 14, or a nucleic acid sequence having at least 80%, 85%, 90%, 91%, 92%, 93%, 94%, 95%, 96%, 97%, 98%, 99% identify to SEQ ID NO: 14.

In some more specific embodiments, provided herein is a vector comprising a nucleic acid sequence of SEQ ID NO: 15, or a nucleic acid sequence having at least 80%, 85%, 90%, 91%, 92%, 93%, 94%, 95%, 96%, 97%, 98%, 99% identify to SEQ ID NO: 15.

In some more specific embodiments, provided herein is a vector comprising a nucleic acid sequence of SEQ ID NO: 16, or a nucleic acid sequence having at least 80%, 85%, 90%, 91%, 92%, 93%, 94%, 95%, 96%, 97%, 98%, 99% identify to SEQ ID NO: 16.

In some more specific embodiments, provided herein is a vector comprising a nucleic acid sequence of SEQ ID NO: 17, or a nucleic acid sequence having at least 80%, 85%, 90%, 91%, 92%, 93%, 94%, 95%, 96%, 97%, 98%, 99% identify to SEQ ID NO: 17.

In some more specific embodiments, provided herein is a vector comprising a nucleic acid sequence of SEQ ID NO: 18, or a nucleic acid sequence having at least 80%, 85%, 90%, 91%, 92%, 93%, 94%, 95%, 96%, 97%, 98%, 99% identify to SEQ ID NO: 18.

In some more specific embodiments, provided herein is a vector comprising a nucleic acid sequence of SEQ ID NO: 19, or a nucleic acid sequence having at least 80%, 85%, 90%, 91%, 92%, 93%, 94%, 95%, 96%, 97%, 98%, 99% identify to SEQ ID NO: 19.

In some more specific embodiments, provided herein is a vector comprising a nucleic acid sequence of SEQ ID NO: 20, or a nucleic acid sequence having at least 80%, 85%, 90%, 91%, 92%, 93%, 94%, 95%, 96%, 97%, 98%, 99% identify to SEQ ID NO: 20.

In some more specific embodiments, provided herein is a vector comprising a nucleic acid sequence of SEQ ID NO: 21, or a nucleic acid sequence having at least 80%, 85%, 90%, 91%, 92%, 93%, 94%, 95%, 96%, 97%, 98%, 99% identify to SEQ ID NO: 21.

In some more specific embodiments, provided herein is a vector comprising a nucleic acid sequence of SEQ ID NO: 22, or a nucleic acid sequence having at least 80%, 85%, 90%, 91%, 92%, 93%, 94%, 95%, 96%, 97%, 98%, 99% identify to SEQ ID NO: 22.

In some more specific embodiments, provided herein is a vector comprising a nucleic acid sequence of SEQ ID NO: 23, or a nucleic acid sequence having at least 80%, 85%, 90%, 91%, 92%, 93%, 94%, 95%, 96%, 97%, 98%, 99% identify to SEQ ID NO: 23.

In some more specific embodiments, provided herein is a vector comprising a nucleic acid sequence of any one of SEQ ID NOs: 67-75, or a nucleic acid sequence having at least 80%, 85%, 90%, 91%, 92%, 93%, 94%, 95%, 96%, 97%, 98%, 99% identify to any one of SEQ ID NOs: 67-75.

In yet another aspect, the rAAV vector provided herein comprises nucleic acids encoding a first domain derived from VEGFR-1 provided herein, a second domain derived from VEGFR-2 provided herein, and a third domain that is capable of binding to an angiopoietin provided herein, each as described in more detail above; and the rAAV vector is constructed in such a way that two or more peptides are expressed instead of a single fusion protein. For example, in some embodiments, the first domain, the second domain and the third domain are each expressed as separate proteins. In other embodiments, the first domain and the second domain are expressed as a single polypeptide, and the third domain is expressed as a second polypeptide.

The methods are known in the art for generating such separately expressed polypeptides via one vector, e.g., via IRES- and 2A peptide-based vector systems or intein mediated protein splicing system. In some embodiments, internal ribosomal entry sites (IRES) are used herein to express multiple genes from one promoter. In other embodiments, 2A self-cleaving peptides are used herein. The members of 2A peptides are named after the virus in which they have been first described. For example, F2A, the first described 2A peptide, is derived from foot-and-mouth disease virus. The self-cleaving 18-22 amino acids long 2A peptides mediate ‘ribosomal skipping’ between the proline and glycine residues and inhibit peptide bond formation without affecting downstream translation. These peptides allow multiple proteins to be encoded as polyproteins, which dissociate into component proteins upon translation. Self-cleaving peptides are found in members of the picornaviridae virus family, including aphthoviruses such as foot-and-mouth disease virus (FMDV), equine rhinitis A virus (ERAV), thosea asigna virus (TaV) and porcine teschovirus-1 (PTV-1) (see Donnelly et al., J. Gen. Virol., 82: 1027-101 (2001); Ryan et al., J. Gen. Virol., 72: 2727-2732 (2001)) and cardioviruses such as theilovirus (e.g., theiler's murine encephalomyelitis) and encephalomyocarditis viruses. The 2A peptides derived from FMDV, ERAV, PTV-1, and TaV are sometimes referred to as “F2A,” “E2A,” “P2A,” and “T2A,” respectively, and are included in the present disclosure, e.g., as described in Donnelly et al., J. Gen. Virol., 78: 13-21 (1997); Ryan and Drew, EMBO J., 13: 928-933 (1994); Szymczak et al., Nature Biotech., 5: 589-594 (2004); Hasegawa et al., Stem Cells, 25(7): 1707-12 (2007). In yet other embodiments, intein mediated protein splicing system is used herein, e.g., as described in Shah and Muir, Chem Sci., 5(1): 446 461 (2014) and Topilina and Mills, Mobile DNA, 5 (5) (2014). Other methods known in the art can also be used in the present constructs.

### 5.3.3. Recombinant AAV Particles

In another aspect, provided herein are recombinant AAVs (rAAVs) or rAAV particles comprising a nucleic acid encoding the fusion protein provided herein, and at least an AAV capsid protein. The nucleic acid includes any rAAV vectors described in Section 5.3.2 above.

The capsid protein may be derived from the same serotype as the ITRs, or a derivative thereof. The capsid may also be of a different serotype than the ITR. For example, in certain embodiments, an AAV particle comprises AAV2 ITRs and an AAV6 capsid (AAV 2/6), AAV2 ITRs and an AAV7 capsid (AAV 2/7), AAV2 ITRs and an AAV8 capsid (AAV 2/8), or AAV2 ITRs and an AAV9 capsid (AAV 2/9).

Naturally occurring AAV capsids comprise AAV VP1, VP2 and VP3 capsid proteins, which are each encoded by splice variants of the AAV cap gene. In general, an AAV particle comprises three proteins. VP1, VP2 and VP3, with VP2 and VP3 being truncated version of VP1 so having sequences that are also comprised by VP1. Generally, the amino acid sequence of VP1 defines the serotype of the capsid. Thus, for example, if the VP1 capsid protein encodes for an AAV2 VP1 protein, AAV will be of the AAV2 serotype, whereas if the VP1 capsid protein encodes an AAV8 VP1 protein, the AAV will be of the AAV8 serotype.

In some embodiments, an AAV capsid protein (e.g., VP1, VP2 and/or VP3) in the present rAAV particle is not a naturally occurring capsid protein. In some embodiments, an AAV capsid protein (e.g., VP1, VP2 and/or VP3) is derived from a naturally occurring capsid protein.

In some embodiments, the AAV capsid protein is a VP1 capsid protein. In other embodiments, the AAV capsid protein is a VP2 capsid protein. In other embodiments, the AAV capsid protein is a VP3 capsid protein. In some embodiments, the rAAV particle comprises a VP1 capsid protein, a VP2 capsid protein and/or a VP3 capsid protein. In other embodiments, the rAAV particle comprises a VP1 capsid protein, a VP2 capsid protein and a VP3 capsid protein. In some embodiments, the rAAV particle comprises a VPI capsid protein, a VP2 capsid protein and/or a VP3 capsid protein, wherein the capsid proteins of the rAAV particle are of the same serotype. In other embodiments, the rAAV particle comprises a VPI capsid protein, a VP2 capsid protein and a VP3 capsid protein, wherein the capsid proteins of the AAV particle are of the same serotype.

In certain aspects, the capsid protein is a variant capsid protein. A variant capsid protein may comprise one or more mutations, e.g. amino acid substitutions, amino acid deletions, and heterologous peptide insertions, compared to a corresponding reference capsid protein such as the naturally occurring parental capsid protein, i.e. the capsid protein from which it was derived. In some embodiments the amino acid sequence of the AAV capsid protein is identical to the amino acid sequence of the wild type, or reference, or parent AAV capsid protein except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues, e.g., except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residue substitutions. In some embodiments, the capsid protein or AAV particle described herein may be a chimeric capsid protein or AAV particle, respectively, comprising a protein sequence of two or more AAV serotype capsid proteins or particles, respectively.

In some embodiments, the capsid protein in the rAAV particle provided herein is derived from an AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9 capsid protein. In specific embodiments, the capsid protein in the rAAV particle provided herein has an amino acid sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% identical to the amino acid sequence of an AAV1, AAV2, AAV2i8, AAV3, AAV3-B. AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9 capsid protein.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV1.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV2.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV2i8.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV3.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99 8% or 100% sequence identity, to any of the VP1, VP2 or VP3 amino acid sequences of AAV3-B.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV4.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV5.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV6.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV7.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV8.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh8.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh8R.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV9.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV10.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh10.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV11.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV12.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV13.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99 8% or 100% sequence identity, to any of the VP1, VP2 or VP3 amino acid sequences of AAV-DJ.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV LK03.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh74.

In certain embodiments, an AAV particle provided herein comprises VP1, VP2 and/or VP3 capsid proteins that comprises a VPI, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV44-9.

In some specific embodiments, the rAAV particle provided herein comprises a nucleic acid encoding the fusion protein provided herein and VP1 of an AAV comprising an amino acid sequence of SEQ ID NO: 43, SEQ ID NO: 44, SEQ ID NO: 45, SEQ ID NO: 46, SEQ ID NO: 47, SEQ ID NO: 48 or SEQ ID NO: 49. In a specific embodiment, the VP1 comprises an amino acid sequence of SEQ ID NO: 43. In a specific embodiment, the VP1 comprises an amino acid sequence of SEQ ID NO: 44. In a specific embodiment, the VP1 comprises an amino acid sequence of SEQ ID NO: 48, which is a variant VP1 of AAV2 comprising amino acid substitutions Y444F, R487G, T491V, Y500F, R585S, R588T, and Y730F. In yet another specific embodiment, the VP1 comprises an amino acid sequence of SEQ ID NO: 49.

TABLE 3
Exemplary VP1, VP2 and VP3 Proteins

| AAV | Sequence | SEQ ID NO |
|---|---|---|
| AAV2 (UniProt: | MAADGYLPDWLEDTLSEGIRQWWKLKPGPPP | SEQ ID NO: 43 |
| P03135-1) | PKPAERHKDDSRGLVLPGYKYLGPFNGLDKG | |
| VP1: amino acids | EPVNEADAAALEHDKAYDRQLDSGDNPYLKY | |
| 1-735 | NHADAEFQERLKEDTSFGGNLGRAVFQAKKR | |
| VP2: amino acids | VLEPLGLVEEPVKTAPGKKRPVEHSPVEPDSSS | |
| 138-735 | GTGKAGQQPARKRLNFGQTGDADSVPDPQPL | |
| VP3: amino acids | GQPPAAPSGLGTNTMATGSGAPMADNNEGAD | |
| 203-735 | GVGNSSGNWHCDSTWMGDRVITTSTRTWALP | |
| | TYNNHLYKQISSQSGASNDNHYFGYSTPWGY | |
| | FDFNRFHCHFSPRDWQRLINNNWGFRPKRLNF | |
| | KLFNIQVKEVTQNDGTTTIANNLTSTVQVFTD | |
| | SEYQLPYVLGSAHQGCLPPFPADVFMVPQYG | |
| | YLTLNNGSQAVGRSSFYCLEYFPSQMLRTGNN | |
| | FTFSYTFEDVPFHSSYAHSQSLDRLMNPLIDQY | |
| | LYYLSRTNTPSGTTTQSRLQFSQAGASDIRDQS | |
| | RNWLPGPCYRQQRVSKTSADNNNSEYSWTGA | |
| | TKYHLNGRDSLVNPGPAMASHKDDEEKFFPQ | |
| | SGVLIFGKQGSEKTNVDIEKVMITDEEEIRTTN | |
| | PVATEQYGSVSTNLQRGNRQAATADVNTQGV | |
| | LPGMVWQDRDVYLQGPIWAKIPHTDGHFHPS | |
| | PLMGGFGLKHPPPQILIKNTPVPANPSTTFSAA | |
| | KFASFITQYSTGQVSVEIEWELQKENSKRWNP | |
| | EIQYTSNYNKSVNVDFTVDINGVYSEPRPIGT | |
| | RYLTRNL | |
| AAV8 | MAADGYLPDWLEDNLSEGIREWWALKPGAP | SEQ ID NO: 44 |
| (Uniprot | KPKANQQKQDDGRGLVLPGYKYLGPFNGLDK | |
| Q8JQF8_9VIRU) | GEPVNAADAAALEHDKAYDQQLQAGDNPYL | |
| VP1: amino acids | RYNHADAEFQERLQEDTSFGGNLGRAVFQAK | |
| 1-738; | KRVLEPLGLVEEGAKTAPGKKRPVEPSPQRSP | |
| VP2: amino acids | DSSTGIGKKGQQPARKRLNFGQTGDSESVPDP | |
| 138-738; | QPLGEPPAAPSGVGPNTMAAGGGAPMADNNE | |
| VP3: amino acids | GADGVGSSSGNWHCDSTWLGDRVITTSTRTW | |
| 204-738. | ALPTYNNHLYKQISNGTSGGATNDNTYFGYST | |
| | PWGYFDFNRFHCHFSPRDWQRLINNNWGFRP | |
| | KRLSFKLFNIQVKEVTQNEGTKTIANNLTSTIQ | |
| | VFTDSEYQLPYVLGSAHQGCLPPFPADVFMIP | |
| | QYGYLTLNNGSQAVGRSSFYCLEYFPSQMLRT | |
| | GNNFQFTYTFEDVPFHSSYAHSQSLDRLMNPL | |
| | IDQYLYYLSRTQTTGGTANTQTLGFSQGGPNT | |
| | MANQAKNWLPGPCYRQQRVSTTTGQNNNSN | |
| | FAWTAGTKYHLNGRNSLANPGIAMATHKDDE | |
| | ERFFPSNGILIFGKQNAARDNADYSDVMLTSE | |
| | EEIKTTNPVATEEYGIVADNLQQQNTAPQIGTV | |
| | NSQGALPGMVWQNRDVYLQGPIWAKIPHTDG | |
| | NFHPSPLMGGFGLKHPPPQILIKNTPVPADPPT | |
| | TFNQSKLNSFITQYSTGQVSVEIEWELQKENSK | |
| | RWNPEIQYTSNYYKSTSVDFAVNTEGVYSEPR | |
| | PIGTRYLTRNL | |
| AAVrh8 | MAADGYLPDWLEDNLSEGIREWWDLKPGAP | SEQ ID NO: 45 |
| (Uniprot | KPKANQQKQDDGRGLVLPGYKYLGPFNGLDK | |
| Q808Y3_9VIRU) | GEPVNAADAAALEHDKAYDQQLKAGDNPYL | |
| VP1: amino acids | RYNHADAEFQERLQEDTSFGGNLGRAVFQAK | |
| 1-736 | KRVLEPLGLVEEGAKTAPGKKRPVEQSPQEPD | |
| VP2: amino acids | SSSGIGKTGQQPAKKRLNFGQTGDSESVPDPQ | |
| 138-736 | PLGEPPAAPSGLGPNTMASGGGAPMADNNEG | |
| VP3: amino acids | ADGVGNSSGNWHCDSTWLGDRVITTSTRTWA | |
| 203-736 | LPTYNNHLYKQISNGTSGGSTNDNTYFGYSTP | |
| | WGYFDFNRFHCHFSPRDWQRLINNNWGFRPK | |
| | RLNFKLFNIQVKEVTTNEGTKTIANNLTSTVQ | |
| | VFTDSEYQLPYVLGSAHQGCLPPFPADVFMVP | |
| | QYGYLTLNNGSQALGRSSFYCLEYFPSQMLRT | |
| | GNNFQFSYTFEDVPFHSSYAHSQSLDRLMNPLI | |
| | DQYLYYLVRTQTTGTGGTQTLAFSQAGPSSM | |
| | ANQARNWVPGPCYRQQRVSTTTNQNNNSNFA | |
| | WTGAAKFKLNGRDSLMNPGVAMASHKDDDD | |
| | RFFPSSGVLIFGKQGAGNDGVDYSQVLITDEEE | |
| | IKATNPVATEEYGAVAINNQAANTQAQTGLV | |
| | HNQGVIPGMVWQNRDVYLQGPIWAKIPHTDG | |
| | NFHPSPLMGGFGLKHPPPQILIKNTPVPADPPL | |
| | TFNQAKLNSFITQYSTGQVSVEIEWELQKENS | |
| | KRWNPEIQYTSNYYKSTNVDFAVNTEGVYSEP | |
| | RPIGTRYLTRNL | |
| AAV9 | MAADGYLPDWLEDNLSEGIREWWALKPGAP | SEQ ID NO: 46 |
| (Uniprot | QPKANQQHQDNARGLVLPGYKYLGPGNGLD | |
| Q6JC40_9VIRU) | KGEPVNAADAAALEHDKAYDQQLKAGDNPY | |
| VP1: amino acids | LKYNHADAEFQERLKEDTSFGGNLGRAVFQA | |
| 1-736 | KKRLLEPLGLVEEAAKTAPGKKRPVEQSPQEP | |
| VP2: amino acids | DSSAGIGKSGAQPAKKRLNFGQTGDTESVPDP | |
| 138-736 | QPIGEPPAAPSGVGSLTMASGGGAPVADNNEG | |
| VP3: amino acids | ADGVGSSSGNWHCDSQWLGDRVITTSTRTWA | |
| 203-736 | LPTYNNHLYKQISNSTSGGSSNDNAYFGYSTP | |
| | WGYFDFNRFHCHFSPRDWQRLINNNWGFRPK | |
| | RLNFKLFNIQVKEVTDNNGVKTIANNLTSTVQ | |
| | VFTDSDYQLPYVLGSAHEGCLPPFPADVFMIP | |
| | QYGYLTLNDGSQAVGRSSFYCLEYFPSQMLRT | |
| | GNNFQFSYEFENVPFHSSYAHSQSLDRLMNPLI | |
| | DQYLYYLSKTINGSGQNQQTLKFSVAGPSNM | |
| | AVQGRNYIPGPSYRQQRVSTTVTQNNNSEFA | |
| | WPGASSWALNGRNSLMNPGPAMASHKEGED | |
| | RFFPLSGSLIFGKQGTGRDNVDADKVMITNEE | |
| | EIKTTNPVATESYGQVATNHQSAQAQAQTGW | |
| | VQNQGILPGMVWQDRDVYLQGPIWAKIPHTD | |
| | GNFHPSPLMGGFGMKHPPPQILIKNTPVPADPP | |
| | TAFNKDKLNSFITQYSTGQVSVEIEWELQKEN | |
| | SKRWNPEIQYTSNYYKSNNVEFAVNTEGVYSE | |
| | PRPIGTRYLTRNL | |
| AAVrh10 | MAADGYLPDWLEDNLSEGIREWWDLKPGAP | SEQ ID NO: 47 |
| (Uniprot | KPKANQQKQDDGRGLVLPGYKYLGPFNGLDK | |
| Q808W5_9VIRU) | GEPVNAADAAALEHDKAYDQQLKAGDNPYL | |
| VP1: amino acids | RYNHADAEFQERLQEDTSFGGNLGRAVFQAK | |
| 1-738; | KRVLEPLGLVEEGAKTAPGKKRPVEPSPQRSP | |
| VP2: amino acids | DSSTGIGKKGQQPAKKRLNFGQTGDSESVPDP | |
| 138-738; | QPIGEPPAGPSGLGSGTMAAGGGAPMADNNE | |
| VP3: amino acids | GADGVGSSSGNWHCDSTWLGDRVITTSTRTW | |
| 204-738. | ALPTYNNHLYKQISNGTSGGSTNDNTYFGYST | |
| | PWGYFDFNRFHCHFSPRDWQRLINNNWGFRP | |
| | KRLNFKLFNIQVKEVTQNEGTKTIANNLTSTIQ | |
| | VFTDSEYQLPYVLGSAHQGCLPPFPADVFMIP | |
| | QYGYLTLNNGSQAVGRSSFYCLEYFPSQMLRT | |
| | GNNFEFSYQFEDVPFHSSYAHSQSLDRLMNPLI | |
| | DQYLYYLSRTQSTGGTAGTQQLLFSQAGPNN | |
| | MSAQAKNWLPGPCYRQQRVSTTLSQNNNSNF | |
| | AWTGATKYHLNGRDSLVNPGVAMATHKDDE | |
| | ERFFPSSGVLMFGKQGAGKDNVDYSSVMLTS | |
| | EEEIKTTNPVATEQYGVVADNLQQQNAAPIVG | |
| | AVNSQGALPGMVWQNRDVYLQGPIWAKIPHT | |
| | DGNFHPSPLMGGFGLKHPPPQILIKNTPVPADP | |
| | PTTFSQAKLASFITQYSTGQVSVEIEWELQKEN | |
| | SKRWNPEIQYTSNYYKSTNVDFAVNTDGTYSE | |
| | PRPIGTRYLTRNL | |
| AAV2v | MAADGYLPDWLEDTLSEGIRQWWKLKPGPPP | SEQ ID NO: 48 |
| VP1: amino acids | PKPAERHKDDSRGLVLPGYKYLGPFNGLDKG | |
| 1-735 | EPVNEADAAALEHDKAYDRQLDSGDNPYLKY | |
| VP2: amino acids | NHADAEFQERLKEDTSFGGNLGRAVFQAKKR | |
| 138-735; | VLEPLGLVEEPVKTAPGKKRPVEHSPVEPDSSS | |
| VP3: amino acids | GTGKAGQQPARKRLNFGQTGDADSVPDPQPL | |
| 204-735 | GQPPAAPSGLGTNTMATGSGAPMADNNEGAD | |
| | GVGNSSGNWHCDSTWMGDRVITTSTRTWALP | |
| | TYNNHLYKQISSQSGASNDNHYFGYSTPWGY | |
| | FDFNRFHCHFSPRDWQRLINNNWGFRPKRLNF | |
| | KLFNIQVKEVTQNDGTTTIANNLTSTVQVFTD | |
| | SEYQLPYVLGSAHQGCLPPFPADVFMVPQYG | |
| | YLTLNNGSQAVGRSSFYCLEYFPSQMLRTGNN | |
| | FTFSYTFEDVPFHSSYAHSQSLDRLMNPLIDQY | |
| | LYFLSRTNTPSGTTTQSRLQFSQAGASDIRDQS | |
| | RNWLPGPCYRQQGVSKVSADNNNSEFSWTGA | |
| | TKYHLNGRDSLVNPGPAMASHKDDEEKFFPQ | |
| | SGVLIFGKQGSEKTNVDIEKVMITDEEEIRTTN | |
| | PVATEQYGSVSTNLQSGNTQAATADVNTQGV | |
| | LPGMVWQDRDVYLQGPIWAKIPHTDGHFHPS | |
| | PLMGGFGLKHPPPQILIKNTPVPANPSTTFSAA | |
| | KFASFITQYSTGQVSVEIEWELQKENSKRWNP | |
| | EIQYTSNYNKSVNVDFTVDTNGVYSEPRPIGT | |
| | RFLTRNL | |
| AAV44-9 | MAADGYLPDWLEDNLSEGIREWWDLKPGAP | SEQ ID NO: 49 |
| VP1: amino acids | KPKANQQKQDDGRGLVLPGYKYLGPFNGLDK | |
| 1-736 | GEPVNAADAAALEHDKAYDQQLKAGDNPYL | |
| VP2: amino acids | RYNHADAEFQERLQEDTSFGGNLGRAVFQAK | |
| 138-736; | KRVLEPLGLVEEGAKTAPGKKRPVEQSPQEPD | |
| VP3: amino acids | SSSGIGKTGQQPAKKRLNFGQTGDTESVPDPQ | |
| 204-736 | PLGEPPAAPSGLGPNTMASGGGAPMADNNEG | |
| | ADGVGNSSGNWHCDSTWLGDRVITTSTRTWA | |
| | LPTYNNHLYKQISNGTSGGSTNDNTYFGYSTP | |
| | WGYFDFNRFHCHFSPRDWQRLINNNWGFRPK | |
| | RLNFKLFNIQVKEVTTNEGTKTIANNLTSTVQ | |
| | VFTDSEYQLPYVLGSAHQGCLPPFPADVFMVP | |
| | QYGYLTLNNGSQALGRSSFYCLEYFPSQMLRT | |
| | GNNFQFSYTFEDVPFHSSYAHSQSLDRLMNPLI | |
| | DQYLYYLVRTQTTGTGGTQTLAFSQAGPSNM | |
| | ASQARNWVPGPSYRQQRVSTTTNQNNNSNFA | |
| | WTGAAKFKLNGRDSLMNPGVAMASHKDDED | |
| | RFFPSSGVLIFGKQGAGNDGVDYSQVLITDEEE | |
| | IKATNPVATEEYGAVAINNQAANTQAQTGLV | |
| | HNQGVIPGMVWQNRDVYLQGPIWAKIPHTDG | |
| | NFHPSPLMGGFGLKHPPPQILIKNTPVPADPPL | |
| | TFNQAKLNSFITQYSTGQVSVEIEWELQKENS | |
| | KRWNPEIQYTSNYYKSTNVDFAVNTEGVYSEP | |
| | RPIGTRYLTRNL | |

The rAAV particles described herein may be produced using any suitable method known in the art. For example, a host cell (e.g., a mammalian cell) may be engineered to stably express the necessary components for AAV particle production. This can be achieved by integrating a plasmid (or multiple plasmids) comprising AAV rep and cap genes, and a selectable marker, such as an antibiotic (e.g., neomycin or ampicillin) resistance gene into the genome of the cell. The cell can be, e.g., an insect or mammalian cell which can then be co-infected with a helper virus (e.g., adenovirus or baculovirus providing the helper functions) and the rAAV vector comprising the 5′ and 3′ AAV ITR. The use of a selectable marker allows for large-scale production of the rAAV. As another non-limiting example, adenovirus or baculovirus rather than plasmids can be used to introduce rep and cap genes into packaging cells. As yet another non-limiting example, both the viral vector containing the 5′ and 3′ AAV ITRs and the rep and cap genes can be stably integrated into the DNA of producer cells, and the helper functions can be provided by a wild-type adenovirus to produce the rAAV.

A helper virus for AAV refers to a virus that allows AAV to be replicated and packaged by a host cell. A helper virus provides helper functions that allow for the replication of AAV. A number of such helper viruses have been identified, including adenoviruses, herpesviruses and poxviruses such as vaccinia. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4orf6 functions.

A preparation of AAV is said to be substantially free of helper virus if the ratio of infectious AAV particles to infectious helper virus particles is at least about 102:1; at least about 104:1, at least about 106:1; or at least about 108:1. Preparations can also be free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

In certain embodiments, host cells containing the rAAV vectors described above is rendered capable of providing AAV helper functions to replicate and encapsulate the polynucleotide encoding the fusion protein provided herein flanked by the AAV ITRs to produce rAAV particles. AAV helper functions are generally AAV-derived coding sequences which can be expressed to provide AAV gene products that, in turn, function in trans for productive AAV replication AAV helper functions are used herein to complement necessary AAV functions that are missing from the rAAV vectors. In some embodiments. AAV helper functions include one, or both of the major AAV ORFs, namely the rep and cap coding regions, or functional homologues thereof.

AAV helper functions can be introduced into the host cell by transfecting the host cell with an AAV helper construct either prior to, or concurrently with, the transfection of the rAAV vector. For example, AAV helper constructs can be used to provide at least transient expression of AAV rep and/or cap genes to complement missing AAV functions that are necessary for productive AAV infection. Typically, AAV helper constructs lack AAV ITRs and can neither replicate nor package themselves. The AAV helper constructs can be in the form of, e.g., a plasmid, phage, transposon, cosmid, virus, or virion.

In certain embodiments, the host cell is also capable of providing or is provided with non AAV-derived functions or “accessory functions” to produce rAAV particles. Accessory functions are non AAV-derived viral and/or cellular functions upon which AAV is dependent for its replication, such as non AAV proteins and RNAs that are required in AAV replication, including those involved in activation of AAV gene transcription, stage specific AAV mRNA splicing, AAV DNA replication, synthesis of Cap expression products and AAV capsid assembly. In some embodiments, viral-based accessory functions can be derived from a known helper virus.

In some embodiments, as a result of the infection of the host cell with a helper virus and/or an accessory function vector, a recombinant AAV particle is produced, and the produced rAAV particle is infectious, replication-defective virus, and includes an AAV protein shell that encapsulates a heterologous nucleotide sequence of interest flanked on both sides by AAV ITRs

rAAV particles can be purified from the host cell using a purification method known in the art, such as chromatography, CsCl gradients, and other methods as described, for example, in U.S. Pat. Nos. 6,989,264 and 8,137,948 and WO 2010/148143. In some embodiments, residual helper virus can be inactivated using known methods, e.g., by heating.

### 5.3.4. Cells

A variety of host cells can be used to produce rAAV particles described herein. Suitable host cells for producing AAV particles from the polynucleotides and AAV vectors provided herein include microorganisms, yeast cells, insect cells, and mammalian cells. Typically, such cells can be, or have been, used as recipients of a heterologous nucleic acid molecule and can grow in, e.g., suspension culture and a bioreactor.

In some embodiments, the cell is a mammalian host cell, for example, a HEK293, HEK293-T, A549, WEHI, 10T1/2, BHK, MDCK, COS1, COS7, BSC 1, BSC 40, BMT 10, VERO, W138, HeLa, 293, Jurkat, 2V6.11, Saos, C2C12, L, HT1080, HepG2, primary fibroblast, hepatocyte, and myoblast cells.

In other embodiments, the cell is an insect cell, for example an Sf9, SF21, SF900+, or a Drosophila cell lines, mosquito cell lines, e.g., Aedes albopictus derived cell lines, domestic silkworm cell lines, e.g. Bombyxmori cell lines, Trichoplusia ni cell lines such as High Five cells or Lepidoptera cell lines such as Ascalapha odorata cell lines. In some embodiments, insect cells are cells from the insect species which are susceptible to baculovirus infection, including High Five, Sf9, Se301, SeIZD2109, SeUCRI, Sf900+, Sf21, BTI-TN-5B1-4, MG-1, Tn368, HzAm1, BM-N, Ha2302, Hz2E5 and Ao38. For example, large scale production of recombinant AAV in cells, including Sf9 insect cells, has been described by Kotin RM. Hum Mol Genet. 20(R1):R2-R6 (2011) doi: 10.1093/hmg/ddr141. Methodology for molecular engineering and expression of polypeptides in insect cells is described, for example, in Summers and Smith. A Manual of Methods for Baculovirus Vectors and Insect Culture Procedures, Texas Agricultural Experimental Station Bull. No. 7555, College Station, Tex. (1986); King, L. A. and R. D. Possee, The baculovirus expression system, Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995).

### 5.4. Polynucleotides

In certain embodiments, the disclosure provides polynucleotides that encode the various fusion proteins provided herein. In exemplary embodiments, the nucleic acid molecule provided herein comprises a sequence that encodes the fusion protein having the sequence of SEQ ID NO: 7. In exemplary embodiments, the nucleic acid molecule provided herein comprises a sequence that encodes the fusion protein having the sequence of SEQ ID NO: 8. In exemplary embodiments, the nucleic acid molecule provided herein comprises a sequence that encodes the fusion protein having the sequence of SEQ ID NO: 9. In exemplary embodiments, the nucleic acid molecule provided herein comprises a sequence that encodes the fusion protein having the sequence of SEQ ID NO: 10. In exemplary embodiments, the nucleic acid molecule provided herein comprises a sequence that encodes the fusion protein having the sequence of SEQ ID NO: 58. In exemplary embodiments, the nucleic acid molecule provided herein comprises a sequence that encodes the fusion protein having the sequence of SEQ ID NO: 59. In exemplary embodiments, the nucleic acid molecule provided herein comprises a sequence that encodes the fusion protein having the sequence of SEQ ID NO: 60. In exemplary embodiments, the nucleic acid molecule provided herein comprises a sequence that encodes the fusion protein having the sequence of SEQ ID NO: 61. In exemplary embodiments, the nucleic acid molecule provided herein comprises a sequence that encodes the fusion protein having the sequence of SEQ ID NO: 62. In exemplary embodiments, the nucleic acid molecule provided herein comprises a sequence that encodes the fusion protein having the sequence of SEQ ID NO: 63. In exemplary embodiments, the nucleic acid molecule provided herein comprises a sequence that encodes the fusion protein having the sequence of SEQ ID NO: 64. In exemplary embodiments, the nucleic acid molecule provided herein comprises a sequence that encodes the fusion protein having the sequence of SEQ ID NO: 65. In exemplary embodiments, the nucleic acid molecule provided herein comprises a sequence that encodes the fusion protein having the sequence of SEQ ID NO: 66.

In some embodiments, the nucleic acid molecule provided herein comprises SEQ ID NO: 67. In some embodiments, the nucleic acid molecule provided herein comprises SEQ ID NO: 68. In some embodiments, the nucleic acid molecule provided herein comprises SEQ ID NO: 69. In some embodiments, the nucleic acid molecule provided herein comprises SEQ ID NO: 70. In some embodiments, the nucleic acid molecule provided herein comprises SEQ ID NO: 71. In some embodiments, the nucleic acid molecule provided herein comprises SEQ ID NO: 72. In some embodiments, the nucleic acid molecule provided herein comprises SEQ ID NO: 73. In some embodiments, the nucleic acid molecule provided herein comprises SEQ ID NO: 74. In some embodiments, the nucleic acid molecule provided herein comprises SEQ ID NO: 75.

In certain embodiments, the disclosure provides polynucleotides of any recombinant vectors provided herein, including, e.g., SEQ ID NOs: 11-23.

The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the fusion proteins of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the fusion protein of the disclosure.

In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide of a vector provided herein.

As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In certain embodiments, a polynucleotide is isolated. In certain embodiments, a polynucleotide is substantially pure.

### 5.5. Pharmaceutical Compositions

In one aspect, the present disclosure further provides pharmaceutical compositions comprising the fusion protein, vector or viral particle of the present disclosure. In some embodiments, a pharmaceutical composition comprises a therapeutically effective amount of the fusion proteins, vectors or viral particles provided herein and a pharmaceutically acceptable excipient.

In some embodiments, provided herein is a pharmaceutical composition comprising a therapeutically effective amount of the fusion protein provided herein and a pharmaceutically acceptable excipient.

In some embodiments, provided herein is a pharmaceutical composition comprising a therapeutically effective amount of the rAAV vectors provided herein and a pharmaceutically acceptable excipient.

In other embodiments, provided herein is a pharmaceutical composition comprising a therapeutically effective amount of the rAAV particles provided herein and a pharmaceutically acceptable excipient.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, PA. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, binding molecule, viral particle, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they are preferably sterile. The pharmaceutical composition may be rendered sterile by filtration through sterile filtration membranes. The pharmaceutical compositions herein generally can be placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, intravitreal, subretinal injection, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see. e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The pharmaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the therapeutic molecule provided herein, construction of a nucleic acid as part of a viral vector or other vector, etc.

In some embodiments, the pharmaceutical composition provided herein contains the binding molecules and/or viral particles in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

In specific embodiments, the pharmaceutical compositions provided herein are suitable for intravitreal or subretinal injection into an eye of a subject.

### 5.6. Methods and Uses

In another aspect, provided herein are methods for using and uses of the fusion proteins, the vectors, or the viral particles (rAAV) provided herein.

Such methods and uses include therapeutic methods and uses, for example, involving administration of the molecules, rAAV or compositions containing the same, to a subject having a disease or disorder. In some embodiments, the molecule, viral particle, and/or composition is administered in an effective amount to effect treatment of the disease or disorder. Uses include uses of the fusion proteins or viral particles in such methods and treatments, and in the preparation of a medicament in order to carry out such therapeutic methods. In some embodiments, the methods are carried out by administering the fusion proteins or viral particles, or compositions comprising the same, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as cancer or an eye disease). This delay can be of varying lengths of time, depending on the history of the disease and/or individual being treated. As is evident to one skilled in the art, a sufficient or significant delay can, in effect, encompass prevention, in that the individual does not develop the disease or disorder.

In other embodiments, the method or the use provided herein prevents a disease or disorder. In some embodiments, the disease or disorder is associated with VEGF and/or angiopoietin. In some embodiments, the disease or disorder is associated with angiogenesis.

The diseases or disorders herein include, but not limited to, inflammatory disease, ocular disease, autoimmune disease, or cancer. In some embodiments, the disease or disorder is selected from a group consisting of rheumatoid arthritis, inflammatory arthritis, osteoarthritis, cancer, age-related macular degeneration (AMD) (such as wet AMD or dry AMD), ocular disease characterized by neovascularization (such as choroidal neovascularization), uveitis (such as anterior uveitis or posterior uveitis), retinitis pigmentosa, and diabetic retinopathy. In some embodiments, the disease or disorder is age-related macular degeneration (AMD) (such as wet AMD or dry AMD).

In some embodiments, treatment using the fusion protein or the rAAV comprising a nucleic acid encoding the fusion protein leads to lower level of lesion than no treatment or treatment using negative control in an CNV animal model. In some embodiments, the CNV animal model is a mouse model. In some embodiments, the lesion is a Grade 3 lesion whereas the angiograms were graded as follows: score 0, no staining; score 1, slightly stained; score 2, moderately stained; and score 3, strongly stained. In some embodiments, treatment using the fusion protein or the rAAV comprising a nucleic acid encoding the fusion protein leads to lower level of lesion than the treatment using a reference agent in an CNV animal model. In some embodiments, the reference agent is an agent treating AMD. In some embodiments, the reference agent is known drug treating AMD. In some embodiments, treatment using the fusion protein or the rAAV comprising a nucleic acid encoding the fusion protein inhibits late stage CNV lesion.

In some embodiments, the fusion protein provided herein binds to VEGF-A. In some embodiments, the fusion protein provided herein binds to Ang2. In some embodiments, the fusion protein provided herein binds to VEGF-C. In some embodiments, the fusion protein provided herein binds to any two of VEGF-A, Ang2 and VEGF-C. In some embodiments, the fusion protein provided herein binds to all three of VEGF-A, Ang2 and VEGF-C.

In some embodiments, the disease or disorder is an autoimmune disease, such as rheumatoid arthritis, multiple sclerosis, systemic lupus erythematosus, vasculitis (inflammation of the blood vessels), polyneuropathy, cutaneous ulceration, visceral infarction, pleuritis, interstitial fibrosis, Caplan's syndrome, pleuropulmonary nodules, pneumonitis, rheumatoid lung disease or arteritis.

In certain embodiments, the disease or disorder is an inflammatory disease, such as inflammatory arthritis, osteoarthritis, psoriasis, chronic inflammation, irritable bowel disease, lung inflammation or asthma.

In some embodiments, the disease or disorder is cancer including blood cancer and solid tumor cancer. In some embodiments, the cancer is prostate cancer, breast cancer, lung cancer, esophageal cancer, colon cancer, rectal cancer, liver cancer, urinary tract cancer (e.g., bladder cancer), kidney cancer, lung cancer (e.g., non-small cell lung cancer), ovarian cancer, cervical cancer, endometrial cancer, pancreatic cancer, stomach cancer, thyroid cancer, skin cancer (e.g., melanoma), hematopoietic cancers of lymphoid or myeloid lineage, head and neck cancer, nasopharyngeal carcinoma (NPC), glioblastoma, teratocarcinoma, neuroblastoma, adenocarcinoma, cancers of mesenchymal origin such as a fibrosarcoma or rhabdomyosarcoma, soft tissue sarcoma and carcinoma, choriocarcinoma, hepatoblastoma, Karposi's sarcoma or Wilm's tumor.

Other diseases that are associated with angiogenesis can be treated with the methods and compositions disclosed herein, including but not limited to, atherosclerosis, retrolentral fibroplasia, thyroid hyperplasias (including grave's disease), nephrotic syndrome, preclampasia, ascites, pericardial effusion (such as associated with pericarditis) and pleural effusion.

In some embodiments, the methods and compositions provided herein can be used to treat an ocular or eye disease or disorder. In some embodiments, the eye disease is uveitis, retinitis pigmentosa, neovascular glaucoma, diabetic retinopathy (DR) (including proliferative diabetic retinopathy), ischemic retinopathy, intraocular neovascularization, age-related macular degeneration (AMD) including wet AMD and dry AMD, retinal neovascularization, diabetic macular edema (DME), diabetic retina ischemia, diabetic retinal edema, retinal vein occlusion (including central retinal vein occlusion and branched retinal vein occlusion), or macular edema, macular edema following retinal vein occlusion (RVO). In some embodiments, the compositions or methods provide herein treat or prevent one or more symptoms of an eye disease including, but not limited to, formation of ocular drusen, inflammation in the eye or eye tissue and loss of vision.

In certain specific embodiments, the methods and compositions provided herein are used to treat AMD. AMD is characterized by progressive loss of central vision which occurs as a result of damage to the photoreceptor cells in an area of the retina called the macula. AMD has been broadly classified into two clinical states: a wet form and a dry form. It is generally accepted that the wet form of AMD is preceded by and arises from the dry form. Dry AMD is characterized by the formation of macular drusen, tiny yellow or white accumulations of extracellular material that builds up between Bruch's membrane and the retinal pigment epithelium of the eye. Wet AMD, which accounts for most of serious vision loss, is associated with neovascularization, wherein blood vessels grow up from the choroid beneath the retina, and with the leakage of these new vessels. The accumulation of blood and fluid can cause retinal detachment followed by rapid photoreceptor degeneration and loss of vision in either form of AMD.

The fusion protein can be delivered to a subject in a composition. The fusion protein can also be delivered to a subject by a rAAV comprising a nucleic acid encoding the fusion protein. Pharmaceutical compositions comprising the fusion protein or the rAAV comprising a nucleic acid encoding the fusion protein are provided herein and described in more detail above.

The pharmaceutical compositions described herein can be administered to an individual by any route, e.g., intravascularly (e.g., intravenously (IV) or intraarterially), directly into arteries, systemically (for example by intravenous injection), or locally (for example by intraarterial or intraocular injection). Non-limiting exemplary administration methods include intravenous (e.g., by infusion pumps), intraperitoneal, intraocular, intra-arterial, intrapulmonary, oral, inhalation, intravesicular, intramuscular, intra-tracheal, subcutaneous, intraocular, intrathecal, transdermal, transpleural, intraarterial, topical, inhalational (e.g., as mists of sprays), mucosal (such as via nasal mucosa), subcutaneous, transdermal, gastrointestinal, intraarticular, intracisternal, intraventricular, intracranial, intraurethral, intrahepatic, intratumoral, intravitreal and subretinal injection.

In some embodiments, the compositions are administered directly to the eye or the eye tissue, e.g., via intravitreal or subretinal injection. In some embodiments, the compositions are administered topically to the eye, for example, in eye drops. In some embodiments, the compositions are administered by injection to the eye (intraocular injection) or to the tissues associated with the eye. The compositions can be administered, e.g., by intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, or posterior juxtascleral delivery.

The compositions may be administered, e.g., to the vitreous, aqueous humor, sclera, conjunctiva, the area between the sclera and conjunctiva, the retina choroids tissues, macula, or other area in or proximate to the eye of an individual. The compositions can also be administered to the individual as an implant, such as biocompatible and/or biodegradable sustained release formulations which gradually release the compounds over a period of time. The compositions can also be administered to the individual using iontophoresis.

The effective amount of the compositions can be determined empirically by those skilled in the art and depends on the type and severity of the disease, route of administration, disease progression and health, etc. For example, when administered intraocularly, a rAAV comprising a nucleic acid encoding a fusion protein described herein can be administered to a subject at a dose of 1×10⁸ to 1×10¹⁵ vector genomes (vg), such as 1×10⁹ to 1×10¹¹ vector genomes (vg) or 1×10¹¹ to 1×10¹³ vector genomes (vg), and including, e.g., 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴ vector genomes (vg).

In some embodiments, an unit dose comprises a volume that is not more than 1 mL. In some embodiments, an unit dose comprises a volume that is not more than 0.5-1.0 mL. In some embodiments, an unit dose comprises a volume that is not more than 0.5 mL. In some embodiments, an unit dose comprises a volume that is not more than 500 μL.

Pharmaceutical compositions comprising a fusion protein may be administered in a single daily dose, or the daily dose may be administered in divided dosages of two, three, or four times daily. Compositions comprising a fusion protein can also be administered multiple times (e.g., twice, three times, four times, or five times) within a time period (e.g., 1 week, 2 weeks, 3 weeks, 1 month, 2 months, 3 months, 4 months, 5 months, 6 months, 7 months, 8 months, 9 months, 10 months, 11 months, 1 year, 2 years, or 3 years).

Pharmaceutical compositions comprising a rAAV comprising a nucleic acid encoding a fusion protein can be administered less frequently, e.g., once every three months, every four months, once every five months, once every six months, once every seven months, once every eight months, once every nine months, once every ten months, once every eleven months, once every year, once every two years, once every three years, once every 4 years, once every 5 years, or even less frequent. In some embodiments, a single dose of a composition comprising a rAAV comprising a nucleic acid encoding a fusion protein described herein is administered.

In some embodiments, the pharmaceutical composition provided herein is a suspension, e.g., a refrigerated suspension. In some embodiments, the method further comprises agitating the suspension to ensure even distribution of the suspension prior to the administration step. In some embodiments, the method further comprises warming the pharmaceutical composition to room temperature prior to the administration step.

The compositions may also be administered in a sustained release formulation. The sustained release devices (such as pellets, nanoparticles, microparticles, nanospheres, microspheres, and the like) may be administered by injection or surgical implanted in various locations in the eye or tissue associated with the eye, such as intraocular, intravitreal, subretinal, periocular, subconjunctival, or sub-tenons.

The fusion protein or the rAAV comprising a nucleic acid encoding the fusion protein or pharmaceutical composition comprising same can be used either alone or in combination with one or more additional therapeutic agents or other therapies. Exemplary additional therapeutic agents include complement inhibitors, anti-angiogenics, and anti-VEGF agents, such as Lucentis®, Avastin®, trebananib, and conbercept. In some embodiments, a combination is provided as a simultaneous administration, wherein a fusion protein or a rAAV comprising a nucleic acid encoding a fusion protein and at least one therapeutic agent is administered together in the same composition or administered simultaneously in different compositions. In some embodiments, a combination is provided as a separate administration, wherein the administration of a fusion protein or a rAAV comprising a nucleic acid encoding a fusion protein can occur prior to, simultaneously, and/or following administration of at least one therapeutic agent.

### 5.8. Kits and Articles of Manufacture

Further provided are kits, unit dosages, and articles of manufacture comprising any of the compositions described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as an eye disease or disorder) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 6. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 6.1. Example 1—Construction of Fusion Proteins Capable of Binding VEGF and an Angiopoietin

Exemplary fusion proteins according to the present disclosure were constructed. FIG. 1 illustrates these exemplary constructs, i.e., exemplary polypeptide 1, exemplary polypeptide 2, exemplary polypeptide 3, and exemplary polypeptide 4, from top to bottom.

Specifically, these fusion proteins comprise three binding domains-one derived from VEGFR-1 (i.e., the IgG-like domain 2 of VEGFR-1), one derived from VEGFR-2 (i.e., the IgG like domain 3 of VEGFR-2), and one domain capable of binding to an angiopoietin (e.g., angiopoietin 1 and angiopoietin 2). An Flt-1 signal domain was also included at the N-terminus in these exemplary constructs. In certain constructs, the fusion protein further comprises an Fc fragment of human IgG. These sequences for these exemplary domains and exemplary fusion proteins are provided in the table below.

TABLE 4
Exemplary Fusion Proteins and Domains Thereof

| Regions | Sequences | SEQ ID NOS |
|---|---|---|
| Exemplary first | SDTGRPFVEMYSEIPEIIHMTEGRELVIPCRVTSPNI | SEQ ID NO: 1 |
| domain derived | TVTLKKFPLDTLIPDGKRIIWDSRKGFIISNATYKEI | |
| from VEGFR-1 | GLLTCEATVNGHLYKTNYLTHRQTNTIID | |
| (D2) | | |
| Exemplary second | VVLSPSHGIELSVGEKLVLNCTARTELNVGIDENW | SEQ ID NO: 2 |
| domain derived | EYPSSKHQHKKLVNRDLKTQSGSEMKKFLSTLTID | |
| from VEGFR-2 | GVTRSDQGLYTCAASSGLMTKKNSTFVRVHE | |
| (D3) | | |
| 14 amino acid | QEECEWDPWTCEHM | SEQ ID NO: 3 |
| sequence in | | |
| exemplary third | | |
| domain (ABD) | | |
| Exemplary third | GGGGGAQQEECEWDPWTCEHMGSGSATGGSGST | SEQ ID NO: 4 |
| domain (ABD) | ASSGSGSATHQEECEWDPWTCEHMLE | |
| Exemplary IgG-Fc | DKTHTCPPCPAPELLGGPSVFLFPPKPKDTLMISRT | SEQ ID NO: 5 |
| (1) | PEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSRDEL | |
| | TKNQVSLTCLVKGFYPSDIAVEWESNGQPENNYK | |
| | TTPPVLDSDGSFFLYSKLTVDKSRWQQGNVFSCSV | |
| | MHEALHNHYTQKSLSLSPGK | |
| Exemplary IgG-Fc | KDKTHTCPPCPAPELLGGPSVFLFPPKPKDTLMISR | SEQ ID NO: 50 |
| (2) | TPEVTCVVVDVSHEDPEVKFNWYVDGVEVHNAK | |
| | TKPREEQYNSTYRVVSVLTVLHQDWLNGKEYKC | |
| | KVSNKALPAPIEKTISKAKGQPREPQVYTLPPSRD | |
| | ELTKNQVSLTCLVKGFYPSDIAVEWESNGQPENNY | |
| | KTTPPVLDSDGSFFLYSKLTVDKSRWQQGNVFSCS | |
| | VMHEALHNHYTQKSLSLSPGK | |
| Flt-1 Signal | MVSYWDTGVLLCALLSCLLLTGSSSG | SEQ ID NO: 6 |
| | MVSYWDTGVLLCALLSCLLLTGSSSGSDTGRPFV | |
| | EMYSEIPEIIHMTEGRELVIPCRVTSPNITVTLKKFP | |
| | LDTLIPDGKRIIWDSRKGFIISNATYKEIGLLTCEAT | |
| | VNGHLYKTNYLTHRQTNTIIDVVLSPSHGIELSVG | |
| | EKLVLNCTARTELNVGIDENWEYPSSKHQHKKLV | |
| | NRDLKTQSGSEMKKFLSTLTIDGVTRSDQGLYTC | |
| | AASSGLMTKKNSTFVRVHEKDKTHTCPPCPAPEL | |
| Exemplary | LGGPSVFLFPPKPKDTLMISRTPEVTCVVVDVSHE | SEQ ID NO: 7 |
| polypeptide 1 | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSRDELTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPGKGGGGGAQQEECEWDPWTCEHMGSGS | |
| | ATGGSGSTASSGSGSATHQEECEWDPWTCEHMLE | |
| Exemplary | MVSYWDTGVLLCALLSCLLLTGSSSGSDTGRPFV | SEQ ID NO: 8 |
| polypeptide 2 | EMYSEIPEIIHMTEGRELVIPCRVTSPNITVTLKKFP | |
| | LDTLIPDGKRIIWDSRKGFIISNATYKEIGLLTCEAT | |
| | VNGHLYKTNYLTHRQTNTIIDVVLSPSHGIELSVG | |
| | EKLVLNCTARTELNVGIDENWEYPSSKHQHKKLV | |
| | NRDLKTQSGSEMKKFLSTLTIDGVTRSDQGLYTC | |
| | AASSGLMTKKNSTFVRVHEKGGGGGSGGGGGAQ | |
| | QEECEWDPWTCEHMGSGSATGGSGSTASSGSGS | |
| | ATHQEECEWDPWTCEHMLEDKTHTCPPCPAPELL | |
| | GGPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTIS | |
| | KAKGQPREPQVYTLPPSRDELTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPGK | |
| Exemplary | MVSYWDTGVLLCALLSCLLLTGSSSGGGGGGSG | SEQ ID NO: 9 |
| polypeptide 3 | GGGGAQQEECEWDPWTCEHMGSGSATGGSGSTA | |
| | SSGSGSATHQEECEWDPWTCEHMLEGGGGGSSD | |
| | TGRPFVEMYSEIPEIIHMTEGRELVIPCRVTSPNITV | |
| | TLKKFPLDTLIPDGKRIIWDSRKGFIISNATYKEIGL | |
| | LTCEATVNGHLYKTNYLTHRQTNTIIDVVLSPSHG | |
| | IELSVGEKLVLNCTARTELNVGIDENWEYPSSKHQ | |
| | HKKLVNRDLKTQSGSEMKKFLSTLTIDGVTRSDQ | |
| | GLYTCAASSGLMTKKNSTFVRVHEKDKTHTCPPC | |
| | PAPELLGGPSVFLFPPKPKDTLMISRTPEVTCVVV | |
| | DVSHEDPEVKFNWYVDGVEVHNAKTKPREEQYN | |
| | STYRVVSVLTVLHQDWLNGKEYKCKVSNKALPA | |
| | PIEKTISKAKGQPREPQVYTLPPSRDELTKNQVSLT | |
| | CLVKGFYPSDIAVEWESNGQPENNYKTTPPVLDS | |
| | DGSFFLYSKLTVDKSRWQQGNVFSCSVMHEALH | |
| | NHYTQKSLSLSPGK | |
| Exemplary | MVSYWDTGVLLCALLSCLLLTGSSSGSDTGRPFV | SEQ ID NO: 10 |
| polypeptide 4 | EMYSEIPEIIHMTEGRELVIPCRVTSPNITVTLKKFP | |
| | LDTLIPDGKRIIWDSRKGFIISNATYKEIGLLTCEAT | |
| | VNGHLYKTNYLTHRQTNTIIDVVLSPSHGIELSVG | |
| | EKLVLNCTARTELNVGIDENWEYPSSKHQHKKLV | |
| | NRDLKTQSGSEMKKFLSTLTIDGVTRSDQGLYTC | |
| | AASSGLMTKKNSTFVRVHEKDKTHTCPPCPAPEL | |
| | LGGPSVGGGGGSGGGGGAQQEECEWDPWTCEH | |
| | MGSGSATGGSGSTASSGSGSATHQEECEWDPWTC | |
| | EHMLE | |

### 6.2. Example 2—Constructions of AAV Vectors Comprising the Nucleic Acid Encoding the Fusion Proteins

Nucleic acid sequences encoding the exemplary fusion proteins described in Section 6.1 were introduced into exemplary rAAV vectors derived from rAAV2 in this example to generate rAAV vectors EXG102-02, EXG102-03-01, EXG102-03-02, EXG102-04, EXG102-05, EXG102-06, EXG102-07, EXG102-08, EXG102-09, EXG102-10, EXG102-11, EXG102-12, and EXG102-13.

Specifically, exemplary polypeptide 1 is the gene of interest in EXG102-04, EXG102-07, and EXG102-08; exemplary polypeptide 2 is the gene of interest in EXG102-09, EXG102-10; exemplary polypeptide 3 is the gene of interest in EXG102-11, EXG102-12. Exemplary polypeptide 4 is the gene of interest in EXG102-13; and a control polypeptide without an Ang BD was constructed into EXG102-02, EXG102-03-1, EXG102-03-2, EXG102-05, and EXG102-06.

These vectors are illustrated in FIG. 2, in which TR represents AAV2 inverted terminal repeats, CBA is the 1.68 kb chicken beta-actin promoter, CB is the 0.78 kb small chicken beta-actin promoter, D2 represents the IgG-like domain 2 of VEGFR-1, D3 represents the IgG like domain 3 of VEGFR-2, ABD (or Ang BD) represents the angiopoietin binding domain, IgG Fc is Fc fragment of human IgG, Fc-Hinger is the N-terminal 21 amino acid of the Fc plus a 6 amino acid GS linker, WPRE represents woodchuck hepatitis virus posttranscriptional regulatory element (600 bp), mWPRE is the mini WPRE (240 bp), SV40pA is simian virus 40 polyadenylation signal, and bGHpA is the bovine growth hormone polyadenylation signal. In these constructs, the small CB promoter are only associated with self-complementary AAV (scAAV).

The nucleic acid sequences of these exemplary rAAV vectors are shown in the table below.

TABLE 5
Exemplary rAAV Vectors

| Name | Sequence | SEQ ID NO |
|---|---|---|
| EXG102-02 | TTGGCCACTCCCTCTCTGCGCGCTCGCTCGCTC | SEQ ID NO: 11 |
| | ACTGAGGCCGGGCGACCAAAGGTCGCCCGAC | |
| | GCCCGGGCTTTGCCCGGGCGGCCTCAGTGAGC | |
| | GAGCGAGCGCGCAGAGAGGGAGTGGCCAACT | |
| | CCATCACTAGGGGTTCCTCAGATCTGAATTCGG | |
| | TACCTAGTTATTAATAGTAATCAATTACGGGGTC | |
| | ATTAGTTCATAGCCCATATATGGAGTTCCGCGTT | |
| | ACATAACTTACGGTAAATGGCCCGCCTGGCTG | |
| | ACCGCCCAACGACCCCCGCCCATTGACGTCAA | |
| | TAATGACGTATGTTCCCATAGTAACGCCAATAG | |
| | GGACTTTCCATTGACGTCAATGGGTGGAGTATT | |
| | TACGGTAAACTGCCCACTTGGCAGTACATCAA | |
| | GTGTATCATATGCCAAGTACGCCCCCTATTGAC | |
| | GTCAATGACGGTAAATGGCCCGCCTGGCATTAT | |
| | GCCCAGTACATGACCTTATGGGACTTTCCTACT | |
| | TGGCAGTACATCTACGTATTAGTCATCGCTATTA | |
| | CCATGGTCGAGGTGAGCCCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | TCGCTGCGCGCTGCCTTCGCCCCGTGCCCCGC | |
| | TCCGCCGCCGCCTCGCGCCGCCCGCCCCGGCT | |
| | CTGACTGACCGCGTTACTCCCACAGGTGAGCG | |
| | GGCGGGACGGCCCTTCTCCTCCGGGCTGTAAT | |
| | TAGCGCTTGGTTTAATGACGGCTTGTTTCTTTT | |
| | CTGTGGCTGCGTGAAAGCCTTGAGGGGCTCCG | |
| | GGAGGGCCCTTTGTGCGGGGGGAGCGGCTCG | |
| | GGGGGTGCGTGCGTGTGTGTGTGCGTGGGGA | |
| | GCGCCGCGTGCGGCTCCGCGCTGCCCGGCGGC | |
| | TGTGAGCGCTGCGGGCGCGGCGCGGGGCTTT | |
| | GTGCGCTCCGCAGTGTGCGCGAGGGGAGCGC | |
| | GGCCGGGGGCGGTGCCCCGCGGTGCGGGGGG | |
| | GGCTGCGAGGGGAACAAAGGCTGCGTGCGGG | |
| | GTGTGTGCGTGGGGGGGTGAGCAGGGGGTGT | |
| | GGGCGCGTCGGTCGGGCTGCAACCCCCCCTGC | |
| | ACCCCCCTCCCCGAGTTGCTGAGCACGGCCCG | |
| | GCTTCGGGTGCGGGGCTCCGTACGGGGCGTGG | |
| | CGCGGGGCTCGCCGTGCCGGGCGGGGGGTGG | |
| | CGGCAGGTGGGGGTGCCGGGCGGGGCGGGGC | |
| | CGCCTCGGGCCGGGGAGGGCTCGGGGGAGGG | |
| | GCGCGGCGGCCCCCGGAGCGCCGGCGGCTGT | |
| | CGAGGCGCGGCGAGCCGCAGCCATTGCCTTTT | |
| | ATGGTAATCGTGCGAGAGGGCGCAGGGACTTC | |
| | CTTTGTCCCAAATCTGTGCGGAGCCGAAATCT | |
| | GGGAGGCGCCGCCGCACCCCCTCTAGCGGGC | |
| | GCGGGGCGAAGCGGTGCGGCGCCGGCAGGAA | |
| | GGAAATGGGCGGGGAGGGCCTTCGTGCGTCG | |
| | CCGCGCCGCCGTCCCCTTCTCCCTCTCCAGCCT | |
| | CGGGGCTGTCCGCGGGGGGACGGCTGCCTTC | |
| | GGGGGGGACGGGGCAGGGCGGGGTTCGGCTT | |
| | CTGGCGTGTGACCGGCGGCTCTAGAGCCTCTG | |
| | CTAACCATGTTCATGCCTTCTTCTTTTTCCTACA | |
| | GCTCCTGGGCAACGTGCTGGTTATTGTGCTGTC | |
| | TCATCATTTTGGCAAAGAATTCCTCGAAGATCT | |
| | AGGCAACGCGTCTCGAGGCGGCCGCCACCAT | |
| | GGTGTCTTATTGGGATACTGGCGTGCTGCTCTG | |
| | TGCCCTCCTGAGTTGCCTGCTCCTGACTGGTTC | |
| | TTCTTCTGGGTCCGATACTGGGCGCCCCTTCGT | |
| | GGAGATGTACTCCGAGATCCCTGAAATCATTC | |
| | ACATGACTGAGGGTCGGGAACTGGTCATCCCA | |
| | TGCCGCGTGACCTCTCCCAACATTACTGTGAC | |
| | CCTGAAGAAATTCCCTCTGGACACCCTCATCC | |
| | CAGATGGGAAGAGGATCATTTGGGACTCAAGA | |
| | AAGGGTTTTATCATCAGCAACGCTACATACAA | |
| | GGAGATTGGCCTGCTCACCTGCGAAGCAACAG | |
| | TGAACGGACACCTGTACAAGACTAATTATCTC | |
| | ACCCATAGACAGACAAACACTATCATTGATGT | |
| | GGTCCTGTCACCAAGCCACGGCATCGAGCTCA | |
| | GCGTCGGTGAAAAGCTGGTGCTCAATTGTACA | |
| | GCCCGGACTGAGCTGAACGTGGGCATTGACTT | |
| | CAATTGGGAATACCCCAGCTCCAAGCACCAGC | |
| | ATAAGAAACTGGTGAACCGCGATCTCAAAACC | |
| | CAGTCCGGATCTGAGATGAAGAAATTTCTGAG | |
| | CACCCTCACAATCGACGGCGTGACACGATCCG | |
| | ATCAGGGACTGTATACTTGCGCCGCTTCTAGTG | |
| | GCCTGATGACCAAGAAAAATAGCACATTCGTC | |
| | AGGGTGCACGAAAAGGACAAAACTCATACCT | |
| | GCCCACCTTGTCCAGCACCAGAGCTGCTCGGA | |
| | GGACCATCCGTGTTCCTGTTTCCACCCAAGCC | |
| | CAAAGATACTCTGATGATTTCACGCACACCCG | |
| | AAGTCACTTGCGTGGTCGTGGACGTGTCCCAC | |
| | GAGGACCCCGAAGTCAAGTTTAACTGGTACGT | |
| | GGACGGCGTCGAGGTGCATAATGCTAAGACAA | |
| | AACCCCGAGAGGAACAGTACAACTCTACCTAT | |
| | AGGGTCGTGAGTGTCCTGACAGTGCTCCACCA | |
| | GGATTGGCTGAACGGAAAGGAGTATAAGTGCA | |
| | AAGTGTCTAATAAGGCACTGCCTGCCCCAATC | |
| | GAGAAAACAATTAGTAAGGCCAAAGGGCAGC | |
| | CCAGAGAACCTCAGGTGTACACTCTGCCTCCA | |
| | TCTCGGGACGAGCTCACTAAGAACCAGGTCA | |
| | GTCTGACCTGTCTCGTGAAAGGGTTCTATCCTA | |
| | GTGATATCGCTGTGGAGTGGGAATCAAATGGT | |
| | CAGCCAGAGAACAATTACAAGACCACACCCC | |
| | CTGTCCTGGACAGCGATGGCTCCTTCTTTCTGT | |
| | ATTCCAAGCTCACCGTGGACAAATCTCGATGG | |
| | CAGCAGGGAAACGTCTTTAGTTGTTCAGTGAT | |
| | GCACGAAGCCCTCCATAACCACTACACTCAGA | |
| | AAAGCCTCAGCCTCAGCCCTGGGAAATGATAA | |
| | GCGGCCGCGCGGATCCAGACATGATAAGATAC | |
| | ATTGATGAGTTTGGACAAACCACAACTAGAAT | |
| | GCAGTGAAAAAAATGCTTTATTTGTGAAATTTG | |
| | TGATGCTATTGCTTTATTTGTAACCATTATAAGC | |
| | TGCAATAAACAAGTTAACAACAACAATTGCAT | |
| | TCATTTTATGTTTCAGGTTCAGGGGGAGGTGTG | |
| | GGAGGTTTTTTAGTCGACTGGGGAGAGATCTG | |
| | AGGAACCCCTAGTGATGGAGTTGGCCACTCCC | |
| | TCTCTGCGCGCTCGCTCGCTCACTGAGGCCGC | |
| | CCGGGCAAAGCCCGGGCGTCGGGCGACCTTT | |
| | GGTCGCCCGGCCTCAGTGAGCGAGCGAGCGC | |
| | GCAGAGAGGGAGTGGCCAAC | |
| EXG102-03-01 | TTGGCCACTCCCTCTCTGCGCGCTCGCTCGCTC | SEQ ID NO: 12 |
| | ACTGAGGCCGGGCGACCAAAGGTCGCCCGAC | |
| | GCCCGGGCTTTGCCCGGGCGGCCTCAGTGAGC | |
| | GAGCGAGCGCGCAGAGAGGGAGTGGCCAACT | |
| | CCATCACTAGGGGTTCCTCAGATCTGAATTCGG | |
| | TACCTAGTTATTAATAGTAATCAATTACGGGGTC | |
| | ATTAGTTCATAGCCCATATATGGAGTTCCGCGTT | |
| | ACATAACTTACGGTAAATGGCCCGCCTGGCTG | |
| | ACCGCCCAACGACCCCCGCCCATTGACGTCAA | |
| | TAATGACGTATGTTCCCATAGTAACGCCAATAG | |
| | GGACTTTCCATTGACGTCAATGGGTGGAGTATT | |
| | TACGGTAAACTGCCCACTTGGCAGTACATCAA | |
| | GTGTATCATATGCCAAGTACGCCCCCTATTGAC | |
| | GTCAATGACGGTAAATGGCCCGCCTGGCATTAT | |
| | GCCCAGTACATGACCTTATGGGACTTTCCTACT | |
| | TGGCAGTACATCTACGTATTAGTCATCGCTATTA | |
| | CCATGGTCGAGGTGAGCCCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGGGGAG | |
| | TCGCTGCGCGCTGCCTTCGCCCCGTGCCCCGC | |
| | TCCGCCGCCGCCTCGCGCCGCCCGCCCCGGCT | |
| | CTGACTGACCGCGTTACTCCCACAGGTGAGCG | |
| | GGCGGGACGGCCCTTCTCCTCCGGGCTGTAAT | |
| | TAGCGCTTGGTTTAATGACGGCTTGTTTCTTTT | |
| | CTGTGGCTGCGTGAAAGCCTTGAGGGGCTCCG | |
| | GGAGGGCCCTTTGTGCGGGGGGAGCGGCTCG | |
| | GGGGGTGCGTGCGTGTGTGTGTGCGTGGGGA | |
| | GCGCCGCGTGCGGCTCCGCGCTGCCCGGCGGC | |
| | TGTGAGCGCTGCGGGCGCGGCGCGGGGCTTT | |
| | GTGCGCTCCGCAGTGTGCGCGAGGGGAGCGC | |
| | GGCCGGGGGCGGTGCCCCGCGGTGCGGGGGG | |
| | GGCTGCGAGGGGAACAAAGGCTGCGTGCGGG | |
| | GTGTGTGCGTGGGGGGGTGAGCAGGGGGTGT | |
| | GGGCGCGTCGGTCGGGCTGCAACCCCCCCTGC | |
| | ACCCCCCTCCCCGAGTTGCTGAGCACGGCCCG | |
| | GCTTCGGGTGCGGGGCTCCGTACGGGGCGTGG | |
| | CGCGGGGCTCGCCGTGCCGGGCGGGGGGTGG | |
| | CGGCAGGTGGGGGTGCCGGGCGGGGCGGGGC | |
| | CGCCTCGGGCCGGGGAGGGCTCGGGGGAGGG | |
| | GCGCGGCGGCCCCCGGAGCGCCGGCGGCTGT | |
| | CGAGGCGCGGCGAGCCGCAGCCATTGCCTTTT | |
| | ATGGTAATCGTGCGAGAGGGCGCAGGGACTTC | |
| | CTTTGTCCCAAATCTGTGCGGAGCCGAAATCT | |
| | GGGAGGCGCCGCCGCACCCCCTCTAGCGGGC | |
| | GCGGGGCGAAGCGGTGCGGCGCCGGCAGGAA | |
| | GGAAATGGGCGGGGAGGGCCTTCGTGCGTCG | |
| | CCGCGCCGCCGTCCCCTTCTCCCTCTCCAGCCT | |
| | CGGGGCTGTCCGCGGGGGGACGGCTGCCTTC | |
| | GGGGGGGACGGGGCAGGGCGGGGTTCGGCTT | |
| | CTGGCGTGTGACCGGCGGCTCTAGAGCCTCTG | |
| | CTAACCATGTTCATGCCTTCTTCTTTTTCCTACA | |
| | GCTCCTGGGCAACGTGCTGGTTATTGTGCTGTC | |
| | TCATCATTTTGGCAAAGAATTCCTCGAAGATCT | |
| | AGGCAACGCGTCTCGAGAGAATTCGCCACCAT | |
| | GGTGAGCTATTGGGATACAGGCGTGCTGCTGT | |
| | GCGCCCTGCTGAGCTGCCTGCTGCTGACCGGC | |
| | AGCAGCAGCGGCAGCGACACCGGCAGACCTT | |
| | TCGTGGAGATGTACAGCGAGATCCCTGAGATC | |
| | ATCCACATGACCGAGGGCAGAGAGCTGGTGAT | |
| | CCCTTGCAGAGTGACCAGCCCTAATATCACCG | |
| | TGACCCTCAAGAAGTTCCCTCTGGATACCCTG | |
| | ATCCCTGACGGCAAGAGAATCATCTGGGACAG | |
| | CAGAAAGGGCTTCATCATCAGCAATGCCACCT | |
| | ACAAGGAGATCGGCCTGCTGACCTGCGAGGC | |
| | CACCGTGAATGGCCACCTGTACAAGACCAATT | |
| | ACCTGACCCACAGACAGACCAATACCATCATC | |
| | GACGTGGTGCTGAGCCCTAGCCACGGCATCGA | |
| | GCTGAGCGTGGGCGAGAAGCTGGTGCTGAATT | |
| | GCACCGCCAGAACCGAGCTGAATGTGGGCATC | |
| | GACTTCAATTGGGAGTACCCTAGCAGCAAGCA | |
| | CCAGCACAAGAAGCTGGTGAATAGAGACCTG | |
| | AAGACCCAGAGCGGCAGCGAGATGAAGAAAT | |
| | TCCTGAGCACCCTGACCATCGACGGCGTGACC | |
| | AGAAGCGACCAGGGCCTGTACACCTGCGCTG | |
| | CCAGCAGCGGCCTGATGACCAAGAAGAATAG | |
| | CACCTTCGTGAGAGTGCACGAGAAGGACAAG | |
| | ACCCACACCTGCCCTCCTTGCCCTGCCCCTGA | |
| | GCTGCTGGGCGGCCCTAGCGTGTTCCTGTTCC | |
| | CTCCTAAGCCTAAGGACACCCTCATGATCAGC | |
| | AGAACCCCTGAGGTGACCTGCGTGGTGGTGG | |
| | ACGTGAGCCACGAGGACCCTGAGGTGAAGTT | |
| | CAATTGGTACGTGGACGGCGTGGAGGTGCACA | |
| | ATGCCAAGACCAAGCCTAGAGAGGAGCAGTA | |
| | CAATAGCACCTACAGAGTGGTGAGCGTGCTGA | |
| | CCGTGCTGCACCAGGACTGGCTGAATGGCAAG | |
| | GAGTACAAGTGCAAGGTGAGCAATAAGGCCCT | |
| | GCCTGCCCCTATCGAGAAGACCATCAGCAAGG | |
| | CCAAGGGCCAGCCTAGAGAGCCTCAGGTGTAC | |
| | ACCCTGCCTCCTAGCAGAGACGAGCTGACCAA | |
| | GAATCAGGTGAGCCTGACCTGCCTGGTGAAGG | |
| | GCTTCTACCCTAGCGACATCGCCGTGGAGTGG | |
| | GAGAGCAATGGCCAGCCTGAGAATAATTACAA | |
| | GACCACCCCTCCTGTGCTGGACAGCGACGGCA | |
| | GCTTCTTCCTGTACAGCAAGCTGACCGTGGAC | |
| | AAGAGCAGATGGCAGCAGGGCAATGTGTTCA | |
| | GCTGCAGCGTGATGCACGAGGCCCTGCACAAT | |
| | CACTACACACAGAAGAGCCTGAGCCTGAGCC | |
| | CTGGCAAGTGATAAGGATATCAAGATCTTGCG | |
| | GCCGCTCGATAATCAACCTCTGGATTACAAAAT | |
| | TTGTGAAAGATTGACTGGTATTCTTAACTATGT | |
| | TGCTCCTTTTACGCTATGTGGATACGCTGCTTT | |
| | AATGCCTTTGTATCATGCTATTGCTTCCCGTATG | |
| | GCTTTCATTTTCTCCTCCTTGTATAAATCCTGGT | |
| | TGCTGTCTCTTTATGAGGAGTTGTGGCCCGTTG | |
| | TCAGGCAACGTGGCGTGGTGTGCACTGTGTTT | |
| | GCTGACGCAACCCCCACTGGTTGGGGCATTGC | |
| | CACCACCTGTCAGCTCCTTTCCGGGACTTTCG | |
| | CTTTCCCCCTCCCTATTGCCACGGCGGAACTCA | |
| | TCGCCGCCTGCCTTGCCCGCTGCTGGACAGGG | |
| | GCTCGGCTGTTGGGCACTGACAATTCCGTGGT | |
| | GTTGTCGGGGAAATCATCGTCCTTTCCTTGGCT | |
| | GCTCGCCTGTGTTGCCACCTGGATTCTGCGCG | |
| | GGACGTCCTTCTGCTACGTCCCTTCGGCCCTC | |
| | AATCCAGCGGACCTTCCTTCCCGCGGCCTGCT | |
| | GCCGGCTCTGCGGCCTCTTCCGCGTCTTCGCC | |
| | TTCGCCCTCAGACGAGTCGGATCTCCCTTTGG | |
| | GCCGCCTCCCCGCATCGATACCGTCGAGGAAG | |
| | CTTAAGCTAGAGCTCGCTGATCAGCCTCGACT | |
| | GTGCCTTCTAGTTGCCAGCCATCTGTTGTTTGC | |
| | CCCTCCCCCGTGCCTTCCTTGACCCTGGAAGG | |
| | TGCCACTCCCACTGTCCTTTCCTAATAAAATGA | |
| | GGAAATTGCATCGCATTGTCTGAGTAGGTGTC | |
| | ATTCTATTCTGGGGGGTGGGGTGGGGCAGGAC | |
| | AGCAAGGGGGAGGATTGGGAAGACAATAGCA | |
| | GGCATGCTGGGGAGAGTCTAGAGTCGACTGGG | |
| | GAGAGATCTGAGGAACCCCTAGTGATGGAGTT | |
| | GGCCACTCCCTCTCTGCGCGCTCGCTCGCTCA | |
| | CTGAGGCCGCCCGGGCAAAGCCCGGGCGTCG | |
| | GGCGACCTTTGGTCGCCCGGCCTCAGTGAGCG | |
| | AGCGAGCGCGCAGAGAGGGAGTGGCCAAC | |
| EXG102-03-2 | TTGGCCACTCCCTCTCTGCGCGCTCGCTCGCTC | SEQ ID NO: 13 |
| | ACTGAGGCCGGGCGACCAAAGGTCGCCCGAC | |
| | GCCCGGGCTTTGCCCGGGCGGCCTCAGTGAGC | |
| | GAGCGAGCGCGCAGAGAGGGAGTGGCCAACT | |
| | CCATCACTAGGGGTTCCTCAGATCTGAATTCGG | |
| | TACCTAGTTATTAATAGTAATCAATTACGGGGTC | |
| | ATTAGTTCATAGCCCATATATGGAGTTCCGCGTT | |
| | ACATAACTTACGGTAAATGGCCCGCCTGGCTG | |
| | ACCGCCCAACGACCCCCGCCCATTGACGTCAA | |
| | TAATGACGTATGTTCCCATAGTAACGCCAATAG | |
| | GGACTTTCCATTGACGTCAATGGGTGGAGTATT | |
| | TACGGTAAACTGCCCACTTGGCAGTACATCAA | |
| | GTGTATCATATGCCAAGTACGCCCCCTATTGAC | |
| | GTCAATGACGGTAAATGGCCCGCCTGGCATTAT | |
| | GCCCAGTACATGACCTTATGGGACTTTCCTACT | |
| | TGGCAGTACATCTACGTATTAGTCATCGCTATTA | |
| | CCATGGTCGAGGTGAGCCCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | TCGCTGCGCGCTGCCTTCGCCCCGTGCCCCGC | |
| | TCCGCCGCCGCCTCGCGCCGCCCGCCCCGGCT | |
| | CTGACTGACCGCGTTACTCCCACAGGTGAGCG | |
| | GGCGGGACGGCCCTTCTCCTCCGGGCTGTAAT | |
| | TAGCGCTTGGTTTAATGACGGCTTGTTTCTTTT | |
| | CTGTGGCTGCGTGAAAGCCTTGAGGGGCTCCG | |
| | GGAGGGCCCTTTGTGCGGGGGGAGCGGCTCG | |
| | GGGGGTGCGTGCGTGTGTGTGTGCGTGGGGA | |
| | GCGCCGCGTGCGGCTCCGCGCTGCCCGGCGGC | |
| | TGTGAGCGCTGCGGGCGCGGCGCGGGGCTTT | |
| | GTGCGCTCCGCAGTGTGCGCGAGGGGAGCGC | |
| | GGCCGGGGGCGGTGCCCCGCGGTGCGGGGGG | |
| | GGCTGCGAGGGGAACAAAGGCTGCGTGCGGG | |
| | GTGTGTGCGTGGGGGGGTGAGCAGGGGGTGT | |
| | GGGCGCGTCGGTCGGGCTGCAACCCCCCCTGC | |
| | ACCCCCCTCCCCGAGTTGCTGAGCACGGCCCG | |
| | GCTTCGGGTGCGGGGCTCCGTACGGGGCGTGG | |
| | CGCGGGGCTCGCCGTGCCGGGCGGGGGGTGG | |
| | CGGCAGGTGGGGGTGCCGGGCGGGGCGGGGC | |
| | CGCCTCGGGCCGGGGAGGGCTCGGGGGAGGG | |
| | GCGCGGCGGCCCCCGGAGCGCCGGCGGCTGT | |
| | CGAGGCGCGGCGAGCCGCAGCCATTGCCTTTT | |
| | ATGGTAATCGTGCGAGAGGGCGCAGGGACTTC | |
| | CTTTGTCCCAAATCTGTGCGGAGCCGAAATCT | |
| | GGGAGGCGCCGCCGCACCCCCTCTAGCGGGC | |
| | GCGGGGCGAAGCGGTGCGGCGCCGGCAGGAA | |
| | GGAAATGGGCGGGGAGGGCCTTCGTGCGTCG | |
| | CCGCGCCGCCGTCCCCTTCTCCCTCTCCAGCCT | |
| | CGGGGCTGTCCGCGGGGGGACGGCTGCCTTC | |
| | GGGGGGGACGGGGCAGGGCGGGGTTCGGCTT | |
| | CTGGCGTGTGACCGGCGGCTCTAGAGCCTCTG | |
| | CTAACCATGTTCATGCCTTCTTCTTTTTCCTACA | |
| | GCTCCTGGGCAACGTGCTGGTTATTGTGCTGTC | |
| | TCATCATTTTGGCAAAGAATTCCTCGAAGATCT | |
| | AGGCAACGCGTCTCGAGAGAATTCGCCACCAT | |
| | GGTGAGCTATTGGGATACAGGCGTGCTGCTGT | |
| | GCGCCCTGCTGAGCTGCCTGCTGCTGACCGGC | |
| | AGCAGCAGCGGCAGCGACACCGGCAGACCTT | |
| | TCGTGGAGATGTACAGCGAGATCCCTGAGATC | |
| | ATCCACATGACCGAGGGCAGAGAGCTGGTGAT | |
| | CCCTTGCAGAGTGACCAGCCCTAATATCACCG | |
| | TGACCCTCAAGAAGTTCCCTCTGGATACCCTG | |
| | ATCCCTGACGGCAAGAGAATCATCTGGGACAG | |
| | CAGAAAGGGCTTCATCATCAGCAATGCCACCT | |
| | ACAAGGAGATCGGCCTGCTGACCTGCGAGGC | |
| | CACCGTGAATGGCCACCTGTACAAGACCAATT | |
| | ACCTGACCCACAGACAGACCAATACCATCATC | |
| | GACGTGGTGCTGAGCCCTAGCCACGGCATCGA | |
| | GCTGAGCGTGGGCGAGAAGCTGGTGCTGAATT | |
| | GCACCGCCAGAACCGAGCTGAATGTGGGCATC | |
| | GACTTCAATTGGGAGTACCCTAGCAGCAAGCA | |
| | CCAGCACAAGAAGCTGGTGAATAGAGACCTG | |
| | AAGACCCAGAGCGGCAGCGAGATGAAGAAAT | |
| | TCCTGAGCACCCTGACCATCGACGGCGTGACC | |
| | AGAAGCGACCAGGGCCTGTACACCTGCGCTG | |
| | CCAGCAGCGGCCTGATGACCAAGAAGAATAG | |
| | CACCTTCGTGAGAGTGCACGAGAAGGACAAG | |
| | ACCCACACCTGCCCTCCTTGCCCTGCCCCTGA | |
| | GCTGCTGGGCGGCCCTAGCGTGTTCCTGTTCC | |
| | CTCCTAAGCCTAAGGACACCCTCATGATCAGC | |
| | AGAACCCCTGAGGTGACCTGCGTGGTGGTGG | |
| | ACGTGAGCCACGAGGACCCTGAGGTGAAGTT | |
| | CAATTGGTACGTGGACGGCGTGGAGGTGCACA | |
| | ATGCCAAGACCAAGCCTAGAGAGGAGCAGTA | |
| | CAATAGCACCTACAGAGTGGTGAGCGTGCTGA | |
| | CCGTGCTGCACCAGGACTGGCTGAATGGCAAG | |
| | GAGTACAAGTGCAAGGTGAGCAATAAGGCCCT | |
| | GCCTGCCCCTATCGAGAAGACCATCAGCAAGG | |
| | CCAAGGGCCAGCCTAGAGAGCCTCAGGTGTAC | |
| | ACCCTGCCTCCTAGCAGAGACGAGCTGACCAA | |
| | GAATCAGGTGAGCCTGACCTGCCTGGTGAAGG | |
| | GCTTCTACCCTAGCGACATCGCCGTGGAGTGG | |
| | GAGAGCAATGGCCAGCCTGAGAATAATTACAA | |
| | GACCACCCCTCCTGTGCTGGACAGCGACGGCA | |
| | GCTTCTTCCTGTACAGCAAGCTGACCGTGGAC | |
| | AAGAGCAGATGGCAGCAGGGCAATGTGTTCA | |
| | GCTGCAGCGTGATGCACGAGGCCCTGCACAAT | |
| | CACTACACACAGAAGAGCCTGAGCCTGAGCC | |
| | CTGGCAAGTGATAAGGATATCAAGATCTTGCG | |
| | GCCGCTCGATAATCAACCTCTGGATTACAAAAT | |
| | TTGTGAAAGATTGACTGGTATTCTTAACTATGT | |
| | TGCTCCTTTTACGCTATGTGGATACGCTGCTTT | |
| | AATGCCTTTGTATCATGCTATTGCTTCCCGTATG | |
| | GCTTTCATTTTCTCCTCCTTGTATAAATCCTGGT | |
| | TGCTGTCTCTTTATGAGGAGTTGTGGCCCGTTG | |
| | TCAGGCAACGTGGCGTGGTGTGCACTGTGTTT | |
| | GCTGACGCAACCCCCACTGGTTGGGGCATTGC | |
| | CACCACCTGTCAGCTCCTTTCCGGGACTTTCG | |
| | CTTTCCCCCTCCCTATTGCCACGGCGGAACTCA | |
| | TCGCCGCCTGCCTTGCCCGCTGCTGGACAGGG | |
| | GCTCGGCTGTTGGGCACTGACAATTCCGTGGT | |
| | GTTGTCGGGGAAATCATCGTCCTTTCCTTGGCT | |
| | GCTCGCCTGTGTTGCCACCTGGATTCTGCGCG | |
| | GGACGTCCTTCTGCTACGTCCCTTCGGCCCTC | |
| | AATCCAGCGGACCTTCCTTCCCGCGGCCTGCT | |
| | GCCGGCTCTGCGGCCTCTTCCGCGTCTTCGCC | |
| | TTCGCCCTCAGACGAGTCGGATCTCCCTTTGG | |
| | GCCGCCTCCCCGCATCGATACCGTCGAGGAAG | |
| | CTTAAGCTAGAGCTCGCTGATCAGCCTCGACT | |
| | GTGCCTTCTAGTTGCCAGCCATCTGTTGTTTGC | |
| | CCCTCCCCCGTGCCTTCCTTGACCCTGGAAGG | |
| | TGCCACTCCCACTGTCCTTTCCTAATAAAATGA | |
| | GGAAATTGCATCGCATTGTCTGAGTAGGTGTC | |
| | ATTCTATTCTGGGGGGTGGGGGGGGCAGGAC | |
| | AGCAAGGGGGAGGATTGGGAAGACAATAGCA | |
| | GGCATGCTGGGGAGAGTCTAGAGTCGACTGGG | |
| | GAGAGATCTGAGGAACCCCTAGTGATGGAGTT | |
| | GGCCACTCCCTCTCTGCGCGCTCGCTCGCTCA | |
| | CTGAGGCCGCCCGGGCAAAGCCCGGGCGTCG | |
| | GGCGACCTTTGGTCGCCCGGCCTCAGTGAGCG | |
| | AGCGAGCGCGCAGAGAGGGAGTGGCCAAC | |
| EXG102-04 | CTGCGCGCTCGCTCGCTCACTGAGGCCGCCCG | SEQ ID NO: 14 |
| | GGCAAAGCCCGGGCGTCGGGCGACCTTTGGT | |
| | CGCCCGGCCTCAGTGAGCGAGCGAGCGCGCA | |
| | GAGAGGGAGTGGAATGCACGCGTGGATCTGA | |
| | GTTCAATTCACGCGTGGTACCTCTGGTCGTTAC | |
| | ATAACTTACGGTAAATGGCCCGCCTGGCTGAC | |
| | CGCCCAACGACCCCCGCCCATTGACGTCAATA | |
| | ATGACGTATGTTCCCATAGTAACGCCAATAGGG | |
| | ACTTTCCATTGACGTCAATGGGTGGAGTATTTA | |
| | CGGTAAACTGCCCACTTGGCAGTACATCAAGT | |
| | GTATCATATGCCAAGTACGCCCCCTATTGACGT | |
| | CAATGACGGTAAATGGCCCGCCTGGCATTATGC | |
| | CCAGTACATGACCTTATGGGACTTTCCTACTTG | |
| | GCAGTACATCTACTCGAGGCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | CGGGATCAGCCACCGCGGTGGCGGCCTAGAGT | |
| | CGACGAGGAACTGAAAAACCAGAAAGTTAAC | |
| | TGGTAAGTTTAGTCTTTTTGTCTTTTATTTCAGG | |
| | TCCCGGATCCGGTGGTGGTGCAAATCAAAGAA | |
| | CTGCTCCTCAGTGGATGTTGCCTTTACTTCTAG | |
| | GCCTGTACGGAAGTGTTACTTCTGCTCTAAAA | |
| | GCTGCGGAATTGTACCCGCGGCCGATCCACCG | |
| | GTCCGGAATTCGCCACCATGGTGTCTTATTGGG | |
| | ATACTGGCGTGCTGCTCTGTGCCCTCCTGAGTT | |
| | GCCTGCTCCTGACTGGTTCTTCTTCTGGGTCCG | |
| | ATACTGGGCGCCCCTTCGTGGAGATGTACTCC | |
| | GAGATCCCTGAAATCATTCACATGACTGAGGG | |
| | TCGGGAACTGGTCATCCCATGCCGCGTGACCT | |
| | CTCCCAACATTACTGTGACCCTGAAGAAATTC | |
| | CCTCTGGACACCCTCATCCCAGATGGGAAGAG | |
| | GATCATTTGGGACTCAAGAAAGGGTTTTATCAT | |
| | CAGCAACGCTACATACAAGGAGATTGGCCTGC | |
| | TCACCTGCGAAGCAACAGTGAACGGACACCT | |
| | GTACAAGACTAATTATCTCACCCATAGACAGAC | |
| | AAACACTATCATTGATGTGGTCCTGTCACCAA | |
| | GCCACGGCATCGAGCTCAGCGTCGGTGAAAA | |
| | GCTGGTGCTCAATTGTACAGCCCGGACTGAGC | |
| | TGAACGTGGGCATTGACTTCAATTGGGAATAC | |
| | CCCAGCTCCAAGCACCAGCATAAGAAACTGGT | |
| | GAACCGCGATCTCAAAACCCAGTCCGGATCTG | |
| | AGATGAAGAAATTTCTGAGCACCCTCACAATC | |
| | GACGGCGTGACACGATCCGATCAGGGACTGTA | |
| | TACTTGCGCCGCTTCTAGTGGCCTGATGACCA | |
| | AGAAAAATAGCACATTCGTCAGGGTGCACGAA | |
| | AAGGACAAAACTCATACCTGCCCACCTTGTCC | |
| | AGCACCAGAGCTGCTCGGAGGACCATCCGTGT | |
| | TCCTGTTTCCACCCAAGCCCAAAGATACTCTG | |
| | ATGATTTCACGCACACCCGAAGTCACTTGCGT | |
| | GGTCGTGGACGTGTCCCACGAGGACCCCGAA | |
| | GTCAAGTTTAACTGGTACGTGGACGGCGTCGA | |
| | GGTGCATAATGCTAAGACAAAACCCCGAGAGG | |
| | AACAGTACAACTCTACCTATAGGGTCGTGAGT | |
| | GTCCTGACAGTGCTCCACCAGGATTGGCTGAA | |
| | CGGAAAGGAGTATAAGTGCAAAGTGTCTAATA | |
| | AGGCACTGCCTGCCCCAATCGAGAAAACAATT | |
| | AGTAAGGCCAAAGGGCAGCCCAGAGAACCTC | |
| | AGGTGTACACTCTGCCTCCATCTCGGGACGAG | |
| | CTCACTAAGAACCAGGTCAGTCTGACCTGTCT | |
| | CGTGAAAGGGTTCTATCCTAGTGATATCGCTGT | |
| | GGAGTGGGAATCAAATGGTCAGCCAGAGAAC | |
| | AATTACAAGACCACACCCCCTGTCCTGGACAG | |
| | CGATGGCTCCTTCTTTCTGTATTCCAAGCTCAC | |
| | CGTGGACAAATCTCGATGGCAGCAGGGAAAC | |
| | GTCTTTAGTTGTTCAGTGATGCACGAAGCCCTC | |
| | CATAACCACTACACTCAGAAAAGCCTCAGCCT | |
| | CAGCCCTGGGAAATGATAAGGATATCAAGATC | |
| | TACAAAGCTTATCGATACCGTCGACTAGAGCTC | |
| | GCTGATCAGCCTCGACTGTGCCTTCTAGTTGCC | |
| | AGCCATCTGTTGTTTGCCCCTCCCCCGTGCCTT | |
| | CCTTGACCCTGGAAGGTGCCACTCCCACTGTC | |
| | CTTTCCTAATAAAATGAGGAAATTGCATCGCAT | |
| | TGTCTGAGTAGGTGTCATTCTATTCTGGGGGGT | |
| | GGGGTGGGGCAGGACAGCAAGGGGGAGGATT | |
| | GGGAAGTCTAGAGCAGGCATGCTGGGGAGAG | |
| | ATCGATCTGAGGAACCCCTAGTGATGGAGTTG | |
| | GCCACTCCCTCTCTGCGCGCTCGCTCGCTCAC | |
| | TGAGGCCGGGCGACCAAAGGTCGCCCGACGC | |
| | CCGGGCTTTGCCCGGGCGGCCTCAGTGAGCGA | |
| | GCGAGCGCGCAGAGAGGGAGTGGCC | |
| EXG102-05 | CTGCGCGCTCGCTCGCTCACTGAGGCCGCCCG | SEQ ID NO: 15 |
| | GGCAAAGCCCGGGCGTCGGGCGACCTTTGGT | |
| | CGCCCGGCCTCAGTGAGCGAGCGAGCGCGCA | |
| | GAGAGGGAGTGGAATGCACGCGTGGATCTGA | |
| | GTTCAATTCACGCGTGGTACCTCTGGTCGTTAC | |
| | ATAACTTACGGTAAATGGCCCGCCTGGCTGAC | |
| | CGCCCAACGACCCCCGCCCATTGACGTCAATA | |
| | ATGACGTATGTTCCCATAGTAACGCCAATAGGG | |
| | ACTTTCCATTGACGTCAATGGGTGGAGTATTTA | |
| | CGGTAAACTGCCCACTTGGCAGTACATCAAGT | |
| | GTATCATATGCCAAGTACGCCCCCTATTGACGT | |
| | CAATGACGGTAAATGGCCCGCCTGGCATTATGC | |
| | CCAGTACATGACCTTATGGGACTTTCCTACTTG | |
| | GCAGTACATCTACTCGAGGCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | CGGGATCAGCCACCGCGGTGGCGGCCTAGAGT | |
| | CGACGAGGAACTGAAAAACCAGAAAGTTAAC | |
| | TGGTAAGTTTAGTCTTTTTGTCTTTTATTTCAGG | |
| | TCCCGGATCCGGTGGTGGTGCAAATCAAAGAA | |
| | CTGCTCCTCAGTGGATGTTGCCTTTACTTCTAG | |
| | GCCTGTACGGAAGTGTTACTTCTGCTCTAAAA | |
| | GCTGCGGAATTGTACCCGCGGCCGATCCACCG | |
| | GTCCGGAATTCGCCACCATGGTGTCTTATTGGG | |
| | ATACTGGCGTGCTGCTCTGTGCCCTCCTGAGTT | |
| | GCCTGCTCCTGACTGGTTCTTCTTCTGGGTCCG | |
| | ATACTGGGCGCCCCTTCGTGGAGATGTACTCC | |
| | GAGATCCCTGAAATCATTCACATGACTGAGGG | |
| | TCGGGAACTGGTCATCCCATGCCGCGTGACCT | |
| | CTCCCAACATTACTGTGACCCTGAAGAAATTC | |
| | CCTCTGGACACCCTCATCCCAGATGGGAAGAG | |
| | GATCATTTGGGACTCAAGAAAGGGTTTTATCAT | |
| | CAGCAACGCTACATACAAGGAGATTGGCCTGC | |
| | TCACCTGCGAAGCAACAGTGAACGGACACCT | |
| | GTACAAGACTAATTATCTCACCCATAGACAGAC | |
| | AAACACTATCATTGATGTGGTCCTGTCACCAA | |
| | GCCACGGCATCGAGCTCAGCGTCGGTGAAAA | |
| | GCTGGTGCTCAATTGTACAGCCCGGACTGAGC | |
| | TGAACGTGGGCATTGACTTCAATTGGGAATAC | |
| | CCCAGCTCCAAGCACCAGCATAAGAAACTGGT | |
| | GAACCGCGATCTCAAAACCCAGTCCGGATCTG | |
| | AGATGAAGAAATTTCTGAGCACCCTCACAATC | |
| | GACGGCGTGACACGATCCGATCAGGGACTGTA | |
| | TACTTGCGCCGCTTCTAGTGGCCTGATGACCA | |
| | AGAAAAATAGCACATTCGTCAGGGTGCACGAA | |
| | AAGGACAAAACTCATACCTGCCCACCTTGTCC | |
| | AGCACCAGAGCTGCTCGGAGGACCATCCGTGT | |
| | TCCTGTTTCCACCCAAGCCCAAAGATACTCTG | |
| | ATGATTTCACGCACACCCGAAGTCACTTGCGT | |
| | GGTCGTGGACGTGTCCCACGAGGACCCCGAA | |
| | GTCAAGTTTAACTGGTACGTGGACGGCGTCGA | |
| | GGTGCATAATGCTAAGACAAAACCCCGAGAGG | |
| | AACAGTACAACTCTACCTATAGGGTCGTGAGT | |
| | GTCCTGACAGTGCTCCACCAGGATTGGCTGAA | |
| | CGGAAAGGAGTATAAGTGCAAAGTGTCTAATA | |
| | AGGCACTGCCTGCCCCAATCGAGAAAACAATT | |
| | AGTAAGGCCAAAGGGCAGCCCAGAGAACCTC | |
| | AGGTGTACACTCTGCCTCCATCTCGGGACGAG | |
| | CTCACTAAGAACCAGGTCAGTCTGACCTGTCT | |
| | CGTGAAAGGGTTCTATCCTAGTGATATCGCTGT | |
| | GGAGTGGGAATCAAATGGTCAGCCAGAGAAC | |
| | AATTACAAGACCACACCCCCTGTCCTGGACAG | |
| | CGATGGCTCCTTCTTTCTGTATTCCAAGCTCAC | |
| | CGTGGACAAATCTCGATGGCAGCAGGGAAAC | |
| | GTCTTTAGTTGTTCAGTGATGCACGAAGCCCTC | |
| | CATAACCACTACACTCAGAAAAGCCTCAGCCT | |
| | CAGCCCTGGGAAATGATAAGGATATCAAGATC | |
| | TACAAAGCTTATCGATACCGTCGACTAGAGCTC | |
| | GCTGATCAGCCTCGACTGTGCCTTCTAGTTGCC | |
| | AGCCATCTGTTGTTTGCCCCTCCCCCGTGCCTT | |
| | CCTTGACCCTGGAAGGTGCCACTCCCACTGTC | |
| | CTTTCCTAATAAAATGAGGAAATTGCATCGCAT | |
| | TGTCTGAGTAGGTGTCATTCTATTCTGGGGGGT | |
| | GGGGTGGGGCAGGACAGCAAGGGGGAGGATT | |
| | GGGAAGTCTAGAGCAGGCATGCTGGGGAGAG | |
| | ATCGATCTGAGGAACCCCTAGTGATGGAGTTG | |
| | GCCACTCCCTCTCTGCGCGCTCGCTCGCTCAC | |
| | TGAGGCCGGGCGACCAAAGGTCGCCCGACGC | |
| | CCGGGCTTTGCCCGGGCGGCCTCAGTGAGCGA | |
| | GCGAGCGCGCAGAGAGGGAGTGGCC | |
| EXG102-06 | CTGCGCGCTCGCTCGCTCACTGAGGCCGCCCG | SEQ ID NO: 16 |
| | GGCAAAGCCCGGGCGTCGGGCGACCTTTGGT | |
| | CGCCCGGCCTCAGTGAGCGAGCGAGCGCGCA | |
| | GAGAGGGAGTGGAATGCACGCGTGGATCTGA | |
| | GTTCAATTCACGCGTGGTACCTCTGGTCGTTAC | |
| | ATAACTTACGGTAAATGGCCCGCCTGGCTGAC | |
| | CGCCCAACGACCCCCGCCCATTGACGTCAATA | |
| | ATGACGTATGTTCCCATAGTAACGCCAATAGGG | |
| | ACTTTCCATTGACGTCAATGGGTGGAGTATTTA | |
| | CGGTAAACTGCCCACTTGGCAGTACATCAAGT | |
| | GTATCATATGCCAAGTACGCCCCCTATTGACGT | |
| | CAATGACGGTAAATGGCCCGCCTGGCATTATGC | |
| | CCAGTACATGACCTTATGGGACTTTCCTACTTG | |
| | GCAGTACATCTACTCGAGGCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | CGGGATCAGCCACCGCGGTGGCGGCCTAGAGT | |
| | CGACGAGGAACTGAAAAACCAGAAAGTTAAC | |
| | TGGTAAGTTTAGTCTTTTTGTCTTTTATTTCAGG | |
| | TCCCGGATCCGGTGGTGGTGCAAATCAAAGAA | |
| | CTGCTCCTCAGTGGATGTTGCCTTTACTTCTAG | |
| | GCCTGTACGGAAGTGTTACTTCTGCTCTAAAA | |
| | GCTGCGGAATTGTACCCGCGGCCGATCCACCG | |
| | GTCCGGAATTCGCCACCATGGTGTCTTATTGGG | |
| | ATACTGGCGTGCTGCTCTGTGCCCTCCTGAGTT | |
| | GCCTGCTCCTGACTGGTTCTTCTTCTGGGTCCG | |
| | ATACTGGGCGCCCCTTCGTGGAGATGTACTCC | |
| | GAGATCCCTGAAATCATTCACATGACTGAGGG | |
| | TCGGGAACTGGTCATCCCATGCCGCGTGACCT | |
| | CTCCCAACATTACTGTGACCCTGAAGAAATTC | |
| | CCTCTGGACACCCTCATCCCAGATGGGAAGAG | |
| | GATCATTTGGGACTCAAGAAAGGGTTTTATCAT | |
| | CAGCAACGCTACATACAAGGAGATTGGCCTGC | |
| | TCACCTGCGAAGCAACAGTGAACGGACACCT | |
| | GTACAAGACTAATTATCTCACCCATAGACAGAC | |
| | AAACACTATCATTGATGTGGTCCTGTCACCAA | |
| | GCCACGGCATCGAGCTCAGCGTCGGTGAAAA | |
| | GCTGGTGCTCAATTGTACAGCCCGGACTGAGC | |
| | TGAACGTGGGCATTGACTTCAATTGGGAATAC | |
| | CCCAGCTCCAAGCACCAGCATAAGAAACTGGT | |
| | GAACCGCGATCTCAAAACCCAGTCCGGATCTG | |
| | AGATGAAGAAATTTCTGAGCACCCTCACAATC | |
| | GACGGCGTGACACGATCCGATCAGGGACTGTA | |
| | TACTTGCGCCGCTTCTAGTGGCCTGATGACCA | |
| | AGAAAAATAGCACATTCGTCAGGGTGCACGAA | |
| | AAGGACAAAACTCATACCTGCCCACCTTGTCC | |
| | AGCACCAGAGCTGCTCGGAGGACCATCCGTGT | |
| | TCCTGTTTCCACCCAAGCCCAAAGATACTCTG | |
| | ATGATTTCACGCACACCCGAAGTCACTTGCGT | |
| | GGTCGTGGACGTGTCCCACGAGGACCCCGAA | |
| | GTCAAGTTTAACTGGTACGTGGACGGCGTCGA | |
| | GGTGCATAATGCTAAGACAAAACCCCGAGAGG | |
| | AACAGTACAACTCTACCTATAGGGTCGTGAGT | |
| | GTCCTGACAGTGCTCCACCAGGATTGGCTGAA | |
| | CGGAAAGGAGTATAAGTGCAAAGTGTCTAATA | |
| | AGGCACTGCCTGCCCCAATCGAGAAAACAATT | |
| | AGTAAGGCCAAAGGGCAGCCCAGAGAACCTC | |
| | AGGTGTACACTCTGCCTCCATCTCGGGACGAG | |
| | CTCACTAAGAACCAGGTCAGTCTGACCTGTCT | |
| | CGTGAAAGGGTTCTATCCTAGTGATATCGCTGT | |
| | GGAGTGGGAATCAAATGGTCAGCCAGAGAAC | |
| | AATTACAAGACCACACCCCCTGTCCTGGACAG | |
| | CGATGGCTCCTTCTTTCTGTATTCCAAGCTCAC | |
| | CGTGGACAAATCTCGATGGCAGCAGGGAAAC | |
| | GTCTTTAGTTGTTCAGTGATGCACGAAGCCCTC | |
| | CATAACCACTACACTCAGAAAAGCCTCAGCCT | |
| | CAGCCCTGGGAAATGATAAGGATATCAAGATC | |
| | TATAATCAACCTCTGGATTACAAAATTTGTGAA | |
| | AGATTGACTGGTATTCTTAACTATGTTGCTCCT | |
| | TTTACGCTATGTGGATACGCTGCTTTAATGCCTT | |
| | TGTATCATGCTATTGCTTCCCGTATGGCTTTCAT | |
| | TTTCTCCTCCTTGTATAAATCCTGGTTAGTTCTT | |
| | GCCACGGCGGAACTCATCGCCGCCTGCCTTGC | |
| | CCGCTGCTGGACAGGGGCTCGGCTGTTGGGCA | |
| | CTGACAATTCCGTGGTGTTAAGCTTATCGATAC | |
| | CGTCGACTAGAGCTCGCTGATCAGCCTCGACT | |
| | GTGCCTTCTAGTTGCCAGCCATCTGTTGTTTGC | |
| | CCCTCCCCCGTGCCTTCCTTGACCCTGGAAGG | |
| | TGCCACTCCCACTGTCCTTTCCTAATAAAATGA | |
| | GGAAATTGCATCGCATTGTCTGAGTAGGTGTC | |
| | ATTCTATTCTGGGGGGTGGGGTGGGGCAGGAC | |
| | AGCAAGGGGGAGGATTGGGAAGTCTAGAGCA | |
| | GGCATGCTGGGGAGAGATCGATCTGAGGAACC | |
| | CCTAGTGATGGAGTTGGCCACTCCCTCTCTGC | |
| | GCGCTCGCTCGCTCACTGAGGCCGGGCGACCA | |
| | AAGGTCGCCCGACGCCCGGGCTTTGCCCGGGC | |
| | GGCCTCAGTGAGCGAGCGAGCGCGCAGAGAG | |
| | GGAGTGGCC | |
| EXG102-07 | CTGCGCGCTCGCTCGCTCACTGAGGCCGCCCG | SEQ ID NO: 17 |
| | GGCAAAGCCCGGGCGTCGGGCGACCTTTGGT | |
| | CGCCCGGCCTCAGTGAGCGAGCGAGCGCGCA | |
| | GAGAGGGAGTGGAATGCACGCGTGGATCTGA | |
| | GTTCAATTCACGCGTGGTACCTCTGGTCGTTAC | |
| | ATAACTTACGGTAAATGGCCCGCCTGGCTGAC | |
| | CGCCCAACGACCCCCGCCCATTGACGTCAATA | |
| | ATGACGTATGTTCCCATAGTAACGCCAATAGGG | |
| | ACTTTCCATTGACGTCAATGGGTGGAGTATTTA | |
| | CGGTAAACTGCCCACTTGGCAGTACATCAAGT | |
| | GTATCATATGCCAAGTACGCCCCCTATTGACGT | |
| | CAATGACGGTAAATGGCCCGCCTGGCATTATGC | |
| | CCAGTACATGACCTTATGGGACTTTCCTACTTG | |
| | GCAGTACATCTACTCGAGGCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | CGGGATCAGCCACCGCGGTGGCGGCCTAGAGT | |
| | CGACGAGGAACTGAAAAACCAGAAAGTTAAC | |
| | TGGTAAGTTTAGTCTTTTTGTCTTTTATTTCAGG | |
| | TCCCGGATCCGGTGGTGGTGCAAATCAAAGAA | |
| | CTGCTCCTCAGTGGATGTTGCCTTTACTTCTAG | |
| | GCCTGTACGGAAGTGTTACTTCTGCTCTAAAA | |
| | GCTGCGGAATTGTACCCGCGGCCGATCCACCG | |
| | GTCCGGAATTCGCCACCATGGTGTCTTATTGGG | |
| | ATACTGGCGTGCTGCTCTGTGCCCTCCTGAGTT | |
| | GCCTGCTCCTGACTGGTTCTTCTTCTGGGTCCG | |
| | ATACTGGGCGCCCCTTCGTGGAGATGTACTCC | |
| | GAGATCCCTGAAATCATTCACATGACTGAGGG | |
| | TCGGGAACTGGTCATCCCATGCCGCGTGACCT | |
| | CTCCCAACATTACTGTGACCCTGAAGAAATTC | |
| | CCTCTGGACACCCTCATCCCAGATGGGAAGAG | |
| | GATCATTTGGGACTCAAGAAAGGGTTTTATCAT | |
| | CAGCAACGCTACATACAAGGAGATTGGCCTGC | |
| | TCACCTGCGAAGCAACAGTGAACGGACACCT | |
| | GTACAAGACTAATTATCTCACCCATAGACAGAC | |
| | AAACACTATCATTGATGTGGTCCTGTCACCAA | |
| | GCCACGGCATCGAGCTCAGCGTCGGTGAAAA | |
| | GCTGGTGCTCAATTGTACAGCCCGGACTGAGC | |
| | TGAACGTGGGCATTGACTTCAATTGGGAATAC | |
| | CCCAGCTCCAAGCACCAGCATAAGAAACTGGT | |
| | GAACCGCGATCTCAAAACCCAGTCCGGATCTG | |
| | AGATGAAGAAATTTCTGAGCACCCTCACAATC | |
| | GACGGCGTGACACGATCCGATCAGGGACTGTA | |
| | TACTTGCGCCGCTTCTAGTGGCCTGATGACCA | |
| | AGAAAAATAGCACATTCGTCAGGGTGCACGAA | |
| | AAGGACAAAACTCATACCTGCCCACCTTGTCC | |
| | AGCACCAGAGCTGCTCGGAGGACCATCCGTGT | |
| | TCCTGTTTCCACCCAAGCCCAAAGATACTCTG | |
| | ATGATTTCACGCACACCCGAAGTCACTTGCGT | |
| | GGTCGTGGACGTGTCCCACGAGGACCCCGAA | |
| | GTCAAGTTTAACTGGTACGTGGACGGCGTCGA | |
| | GGTGCATAATGCTAAGACAAAACCCCGAGAGG | |
| | AACAGTACAACTCTACCTATAGGGTCGTGAGT | |
| | GTCCTGACAGTGCTCCACCAGGATTGGCTGAA | |
| | CGGAAAGGAGTATAAGTGCAAAGTGTCTAATA | |
| | AGGCACTGCCTGCCCCAATCGAGAAAACAATT | |
| | AGTAAGGCCAAAGGGCAGCCCAGAGAACCTC | |
| | AGGTGTACACTCTGCCTCCATCTCGGGACGAG | |
| | CTCACTAAGAACCAGGTCAGTCTGACCTGTCT | |
| | CGTGAAAGGGTTCTATCCTAGTGATATCGCTGT | |
| | GGAGTGGGAATCAAATGGTCAGCCAGAGAAC | |
| | AATTACAAGACCACACCCCCTGTCCTGGACAG | |
| | CGATGGCTCCTTCTTTCTGTATTCCAAGCTCAC | |
| | CGTGGACAAATCTCGATGGCAGCAGGGAAAC | |
| | GTCTTTAGTTGTTCAGTGATGCACGAAGCCCTC | |
| | CATAACCACTACACTCAGAAAAGCCTCAGCCT | |
| | CAGCCCTGGGAAAGGCGGCGGCGGAGGCGCC | |
| | CAGCAAGAAGAGTGCGAGTGGGATCCCTGGA | |
| | CCTGCGAGCACATGGGATCCGGCAGCGCCACC | |
| | GGAGGATCCGGAAGCACCGCCTCCAGCGGCT | |
| | CCGGCAGCGCCACCCACCAGGAGGAGTGTGA | |
| | GTGGGACCCCTGGACCTGCGAACACATGCTGG | |
| | AGTGATAAGGATATCAAGATCTATAATCAACCT | |
| | CTGGATTACAAAATTTGTGAAAGATTGACTGG | |
| | TATTCTTAACTATGTTGCTCCTTTTACGCTATGT | |
| | GGATACGCTGCTTTAATGCCTTTGTATCATGCTA | |
| | TTGCTTCCCGTATGGCTTTCATTTTCTCCTCCTT | |
| | GTATAAATCCTGGTTAGTTCTTGCCACGGCGGA | |
| | ACTCATCGCCGCCTGCCTTGCCCGCTGCTGGA | |
| | CAGGGGCTCGGCTGTTGGGCACTGACAATTCC | |
| | GTGGTGTTAAGCTTATCGATACCGTCGACTAGA | |
| | GCTCGCTGATCAGCCTCGACTGTGCCTTCTAGT | |
| | TGCCAGCCATCTGTTGTTTGCCCCTCCCCCGTG | |
| | CCTTCCTTGACCCTGGAAGGTGCCACTCCCAC | |
| | TGTCCTTTCCTAATAAAATGAGGAAATTGCATC | |
| | GCATTGTCTGAGTAGGTGTCATTCTATTCTGGG | |
| | GGGTGGGGTGGGGCAGGACAGCAAGGGGGA | |
| | GGATTGGGAAGTCTAGAGCAGGCATGCTGGGG | |
| | AGAGATCGATCTGAGGAACCCCTAGTGATGGA | |
| | GTTGGCCACTCCCTCTCTGCGCGCTCGCTCGC | |
| | TCACTGAGGCCGGGCGACCAAAGGTCGCCCG | |
| | ACGCCCGGGCTTTGCCCGGGCGGCCTCAGTGA | |
| | GCGAGCGAGCGCGCAGAGAGGGAGTGGCC | |
| EXG102-08 | CTGCGCGCTCGCTCGCTCACTGAGGCCGCCCG | SEQ ID NO: 18 |
| | GGCAAAGCCCGGGCGTCGGGCGACCTTTGGT | |
| | CGCCCGGCCTCAGTGAGCGAGCGAGCGCGCA | |
| | GAGAGGGAGTGGAATGCACGCGTGGATCTGA | |
| | GTTCAATTCACGCGTGGTACCTCTGGTCGTTAC | |
| | ATAACTTACGGTAAATGGCCCGCCTGGCTGAC | |
| | CGCCCAACGACCCCCGCCCATTGACGTCAATA | |
| | ATGACGTATGTTCCCATAGTAACGCCAATAGGG | |
| | ACTTTCCATTGACGTCAATGGGTGGAGTATTTA | |
| | CGGTAAACTGCCCACTTGGCAGTACATCAAGT | |
| | GTATCATATGCCAAGTACGCCCCCTATTGACGT | |
| | CAATGACGGTAAATGGCCCGCCTGGCATTATGC | |
| | CCAGTACATGACCTTATGGGACTTTCCTACTTG | |
| | GCAGTACATCTACTCGAGGCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | CGGGATCAGCCACCGCGGTGGCGGCCTAGAGT | |
| | CGACGAGGAACTGAAAAACCAGAAAGTTAAC | |
| | TGGTAAGTTTAGTCTTTTTGTCTTTTATTTCAGG | |
| | TCCCGGATCCGGTGGTGGTGCAAATCAAAGAA | |
| | CTGCTCCTCAGTGGATGTTGCCTTTACTTCTAG | |
| | GCCTGTACGGAAGTGTTACTTCTGCTCTAAAA | |
| | GCTGCGGAATTGTACCCGCGGCCGATCCACCG | |
| | GTCCGGAATTCGCCACCATGGTGTCTTATTGGG | |
| | ATACTGGCGTGCTGCTCTGTGCCCTCCTGAGTT | |
| | GCCTGCTCCTGACTGGTTCTTCTTCTGGGTCCG | |
| | ATACTGGGCGCCCCTTCGTGGAGATGTACTCC | |
| | GAGATCCCTGAAATCATTCACATGACTGAGGG | |
| | TCGGGAACTGGTCATCCCATGCCGCGTGACCT | |
| | CTCCCAACATTACTGTGACCCTGAAGAAATTC | |
| | CCTCTGGACACCCTCATCCCAGATGGGAAGAG | |
| | GATCATTTGGGACTCAAGAAAGGGTTTTATCAT | |
| | CAGCAACGCTACATACAAGGAGATTGGCCTGC | |
| | TCACCTGCGAAGCAACAGTGAACGGACACCT | |
| | GTACAAGACTAATTATCTCACCCATAGACAGAC | |
| | AAACACTATCATTGATGTGGTCCTGTCACCAA | |
| | GCCACGGCATCGAGCTCAGCGTCGGTGAAAA | |
| | GCTGGTGCTCAATTGTACAGCCCGGACTGAGC | |
| | TGAACGTGGGCATTGACTTCAATTGGGAATAC | |
| | CCCAGCTCCAAGCACCAGCATAAGAAACTGGT | |
| | GAACCGCGATCTCAAAACCCAGTCCGGATCTG | |
| | AGATGAAGAAATTTCTGAGCACCCTCACAATC | |
| | GACGGCGTGACACGATCCGATCAGGGACTGTA | |
| | TACTTGCGCCGCTTCTAGTGGCCTGATGACCA | |
| | AGAAAAATAGCACATTCGTCAGGGTGCACGAA | |
| | AAGGACAAAACTCATACCTGCCCACCTTGTCC | |
| | AGCACCAGAGCTGCTCGGAGGACCATCCGTGT | |
| | TCCTGTTTCCACCCAAGCCCAAAGATACTCTG | |
| | ATGATTTCACGCACACCCGAAGTCACTTGCGT | |
| | GGTCGTGGACGTGTCCCACGAGGACCCCGAA | |
| | GTCAAGTTTAACTGGTACGTGGACGGCGTCGA | |
| | GGTGCATAATGCTAAGACAAAACCCCGAGAGG | |
| | AACAGTACAACTCTACCTATAGGGTCGTGAGT | |
| | GTCCTGACAGTGCTCCACCAGGATTGGCTGAA | |
| | CGGAAAGGAGTATAAGTGCAAAGTGTCTAATA | |
| | AGGCACTGCCTGCCCCAATCGAGAAAACAATT | |
| | AGTAAGGCCAAAGGGCAGCCCAGAGAACCTC | |
| | AGGTGTACACTCTGCCTCCATCTCGGGACGAG | |
| | CTCACTAAGAACCAGGTCAGTCTGACCTGTCT | |
| | CGTGAAAGGGTTCTATCCTAGTGATATCGCTGT | |
| | GGAGTGGGAATCAAATGGTCAGCCAGAGAAC | |
| | AATTACAAGACCACACCCCCTGTCCTGGACAG | |
| | CGATGGCTCCTTCTTTCTGTATTCCAAGCTCAC | |
| | CGTGGACAAATCTCGATGGCAGCAGGGAAAC | |
| | GTCTTTAGTTGTTCAGTGATGCACGAAGCCCTC | |
| | CATAACCACTACACTCAGAAAAGCCTCAGCCT | |
| | CAGCCCTGGGAAAGGCGGCGGCGGAGGCGCC | |
| | CAGCAAGAAGAGTGCGAGTGGGATCCCTGGA | |
| | CCTGCGAGCACATGGGATCCGGCAGCGCCACC | |
| | GGAGGATCCGGAAGCACCGCCTCCAGCGGCT | |
| | CCGGCAGCGCCACCCACCAGGAGGAGTGTGA | |
| | GTGGGACCCCTGGACCTGCGAACACATGCTGG | |
| | AGTGATAAGGATATCAAGATCTACAAAGCTTAT | |
| | CGATACCGTCGACTAGAGCTCGCTGATCAGCC | |
| | TCGACTGTGCCTTCTAGTTGCCAGCCATCTGTT | |
| | GTTTGCCCCTCCCCCGTGCCTTCCTTGACCCTG | |
| | GAAGGTGCCACTCCCACTGTCCTTTCCTAATAA | |
| | AATGAGGAAATTGCATCGCATTGTCTGAGTAG | |
| | GTGTCATTCTATTCTGGGGGGTGGGGTGGGGC | |
| | AGGACAGCAAGGGGGAGGATTGGGAAGTCTA | |
| | GAGCAGGCATGCTGGGGAGAGATCGATCTGAG | |
| | GAACCCCTAGTGATGGAGTTGGCCACTCCCTC | |
| | TCTGCGCGCTCGCTCGCTCACTGAGGCCGGGC | |
| | GACCAAAGGTCGCCCGACGCCCGGGCTTTGCC | |
| | CGGGCGGCCTCAGTGAGCGAGCGAGCGCGCA | |
| | GAGAGGGAGTGGCC | |
| EXG102-09 | TTGGCCACTCCCTCTCTGCGCGCTCGCTCGCTC | SEQ ID NO: 19 |
| | ACTGAGGCCGGGCGACCAAAGGTCGCCCGAC | |
| | GCCCGGGCTTTGCCCGGGCGGCCTCAGTGAGC | |
| | GAGCGAGCGCGCAGAGAGGGAGTGGCCAACT | |
| | CCATCACTAGGGGTTCCTCAGATCTGAATTCGG | |
| | TACCTAGTTATTAATAGTAATCAATTACGGGGTC | |
| | ATTAGTTCATAGCCCATATATGGAGTTCCGCGTT | |
| | ACATAACTTACGGTAAATGGCCCGCCTGGCTG | |
| | ACCGCCCAACGACCCCCGCCCATTGACGTCAA | |
| | TAATGACGTATGTTCCCATAGTAACGCCAATAG | |
| | GGACTTTCCATTGACGTCAATGGGTGGAGTATT | |
| | TACGGTAAACTGCCCACTTGGCAGTACATCAA | |
| | GTGTATCATATGCCAAGTACGCCCCCTATTGAC | |
| | GTCAATGACGGTAAATGGCCCGCCTGGCATTAT | |
| | GCCCAGTACATGACCTTATGGGACTTTCCTACT | |
| | TGGCAGTACATCTACGTATTAGTCATCGCTATTA | |
| | CCATGGTCGAGGTGAGCCCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | TCGCTGCGCGCTGCCTTCGCCCCGTGCCCCGC | |
| | TCCGCCGCCGCCTCGCGCCGCCCGCCCCGGCT | |
| | CTGACTGACCGCGTTACTCCCACAGGTGAGCG | |
| | GGCGGGACGGCCCTTCTCCTCCGGGCTGTAAT | |
| | TAGCGCTTGGTTTAATGACGGCTTGTTTCTTTT | |
| | CTGTGGCTGCGTGAAAGCCTTGAGGGGCTCCG | |
| | GGAGGGCCCTTTGTGCGGGGGGAGCGGCTCG | |
| | GGGGGTGCGTGCGTGTGTGTGTGCGTGGGGA | |
| | GCGCCGCGTGCGGCTCCGCGCTGCCCGGCGGC | |
| | TGTGAGCGCTGCGGGCGCGGCGCGGGGCTTT | |
| | GTGCGCTCCGCAGTGTGCGCGAGGGGAGCGC | |
| | GGCCGGGGGCGGTGCCCCGCGGTGCGGGGGG | |
| | GGCTGCGAGGGGAACAAAGGCTGCGTGCGGG | |
| | GTGTGTGCGTGGGGGGGTGAGCAGGGGGTGT | |
| | GGGCGCGTCGGTCGGGCTGCAACCCCCCCTGC | |
| | ACCCCCCTCCCCGAGTTGCTGAGCACGGCCCG | |
| | GCTTCGGGTGCGGGGCTCCGTACGGGGCGTGG | |
| | CGCGGGGCTCGCCGTGCCGGGCGGGGGGTGG | |
| | CGGCAGGTGGGGGTGCCGGGCGGGGCGGGGC | |
| | CGCCTCGGGCCGGGGAGGGCTCGGGGGAGGG | |
| | GCGCGGCGGCCCCCGGAGCGCCGGCGGCTGT | |
| | CGAGGCGCGGCGAGCCGCAGCCATTGCCTTTT | |
| | ATGGTAATCGTGCGAGAGGGCGCAGGGACTTC | |
| | CTTTGTCCCAAATCTGTGCGGAGCCGAAATCT | |
| | GGGAGGCGCCGCCGCACCCCCTCTAGCGGGC | |
| | GCGGGGCGAAGCGGTGCGGCGCCGGCAGGAA | |
| | GGAAATGGGCGGGGAGGGCCTTCGTGCGTCG | |
| | CCGCGCCGCCGTCCCCTTCTCCCTCTCCAGCCT | |
| | CGGGGCTGTCCGCGGGGGGACGGCTGCCTTC | |
| | GGGGGGGACGGGGCAGGGCGGGGTTCGGCTT | |
| | CTGGCGTGTGACCGGCGGCTCTAGAGCCTCTG | |
| | CTAACCATGTTCATGCCTTCTTCTTTTTCCTACA | |
| | GCTCCTGGGCAACGTGCTGGTTATTGTGCTGTC | |
| | TCATCATTTTGGCAAAGAATTCCTCGAAGATCT | |
| | AGGCAACGCGTCTCGAACGCGTCTCGAGAGA | |
| | ATTCGCCACCATGGTGTCTTATTGGGATACTGG | |
| | CGTGCTGCTCTGTGCCCTCCTGAGTTGCCTGCT | |
| | CCTGACTGGTTCTTCTTCTGGGTCCGATACTGG | |
| | GCGCCCCTTCGTGGAGATGTACTCCGAGATCC | |
| | CTGAAATCATTCACATGACTGAGGGTCGGGAA | |
| | CTGGTCATCCCATGCCGCGTGACCTCTCCCAA | |
| | CATTACTGTGACCCTGAAGAAATTCCCTCTGG | |
| | ACACCCTCATCCCAGATGGGAAGAGGATCATT | |
| | TGGGACTCAAGAAAGGGTTTTATCATCAGCAA | |
| | CGCTACATACAAGGAGATTGGCCTGCTCACCT | |
| | GCGAAGCAACAGTGAACGGACACCTGTACAA | |
| | GACTAATTATCTCACCCATAGACAGACAAACA | |
| | CTATCATTGATGTGGTCCTGTCACCAAGCCACG | |
| | GCATCGAGCTCAGCGTCGGTGAAAAGCTGGTG | |
| | CTCAATTGTACAGCCCGGACTGAGCTGAACGT | |
| | GGGCATTGACTTCAATTGGGAATACCCCAGCT | |
| | CCAAGCACCAGCATAAGAAACTGGTGAACCG | |
| | CGATCTCAAAACCCAGTCCGGATCTGAGATGA | |
| | AGAAATTTCTGAGCACCCTCACAATCGACGGC | |
| | GTGACACGATCCGATCAGGGACTGTATACTTG | |
| | CGCCGCTTCTAGTGGCCTGATGACCAAGAAAA | |
| | ATAGCACATTCGTCAGGGTGCACGAAAAGGGC | |
| | GGCGGCGGAGGCAGCGGCGGCGGCGGAGGCG | |
| | CCCAGCAAGAAGAGTGCGAGTGGGATCCCTG | |
| | GACCTGCGAGCACATGGGATCCGGCAGCGCCA | |
| | CCGGAGGATCCGGAAGCACCGCCTCCAGCGG | |
| | CTCCGGCAGCGCCACCCACCAGGAGGAGTGT | |
| | GAGTGGGACCCCTGGACCTGCGAACACATGCT | |
| | GGAGGACAAAACTCATACCTGCCCACCTTGTC | |
| | CAGCACCAGAGCTGCTCGGAGGACCATCCGTG | |
| | TTCCTGTTTCCACCCAAGCCCAAAGATACTCT | |
| | GATGATTTCACGCACACCCGAAGTCACTTGCG | |
| | TGGTCGTGGACGTGTCCCACGAGGACCCCGA | |
| | AGTCAAGTTTAACTGGTACGTGGACGGCGTCG | |
| | AGGTGCATAATGCTAAGACAAAACCCCGAGAG | |
| | GAACAGTACAACTCTACCTATAGGGTCGTGAG | |
| | TGTCCTGACAGTGCTCCACCAGGATTGGCTGA | |
| | ACGGAAAGGAGTATAAGTGCAAAGTGTCTAAT | |
| | AAGGCACTGCCTGCCCCAATCGAGAAAACAAT | |
| | TAGTAAGGCCAAAGGGCAGCCCAGAGAACCT | |
| | CAGGTGTACACTCTGCCTCCATCTCGGGACGA | |
| | GCTCACTAAGAACCAGGTCAGTCTGACCTGTC | |
| | TCGTGAAAGGGTTCTATCCTAGTGATATCGCTG | |
| | TGGAGTGGGAATCAAATGGTCAGCCAGAGAA | |
| | CAATTACAAGACCACACCCCCTGTCCTGGACA | |
| | GCGATGGCTCCTTCTTTCTGTATTCCAAGCTCA | |
| | CCGTGGACAAATCTCGATGGCAGCAGGGAAA | |
| | CGTCTTTAGTTGTTCAGTGATGCACGAAGCCCT | |
| | CCATAACCACTACACTCAGAAAAGCCTCAGCC | |
| | TCAGCCCTGGGAAATGATAAGCGGCCGCAAGC | |
| | TTAAGAGCTTAAGCTAGAGCTCGCTGATCAGC | |
| | CTCGACTGTGCCTTCTAGTTGCCAGCCATCTGT | |
| | TGTTTGCCCCTCCCCCGTGCCTTCCTTGACCCT | |
| | GGAAGGTGCCACTCCCACTGTCCTTTCCTAATA | |
| | AAATGAGGAAATTGCATCGCATTGTCTGAGTA | |
| | GGTGTCATTCTATTCTGGGGGGTGGGGTGGGG | |
| | CAGGACAGCAAGGGGGAGGATTGGGAAGACA | |
| | ATAGCAGGCATGCTGGGGAGAGTCTAGAGTCG | |
| | ACTGGGGAGAGATCTGAGGAACCCCTAGTGAT | |
| | GGAGTTGGCCACTCCCTCTCTGCGCGCTCGCT | |
| | CGCTCACTGAGGCCGCCCGGGCAAAGCCCGG | |
| | GCGTCGGGCGACCTTTGGTCGCCCGGCCTCAG | |
| | TGAGCGAGCGAGCGCGCAGAGAGGGAGTGGC | |
| | CAAC | |
| EXG102-10 | CTGCGCGCTCGCTCGCTCACTGAGGCCGCCCG | SEQ ID NO: 20 |
| | GGCAAAGCCCGGGCGTCGGGCGACCTTTGGT | |
| | CGCCCGGCCTCAGTGAGCGAGCGAGCGCGCA | |
| | GAGAGGGAGTGGAATGCACGCGTGGATCTGA | |
| | GTTCAATTCACGCGTGGTACCTCTGGTCGTTAC | |
| | ATAACTTACGGTAAATGGCCCGCCTGGCTGAC | |
| | CGCCCAACGACCCCCGCCCATTGACGTCAATA | |
| | ATGACGTATGTTCCCATAGTAACGCCAATAGGG | |
| | ACTTTCCATTGACGTCAATGGGTGGAGTATTTA | |
| | CGGTAAACTGCCCACTTGGCAGTACATCAAGT | |
| | GTATCATATGCCAAGTACGCCCCCTATTGACGT | |
| | CAATGACGGTAAATGGCCCGCCTGGCATTATGC | |
| | CCAGTACATGACCTTATGGGACTTTCCTACTTG | |
| | GCAGTACATCTACTCGAGGCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | CGGGATCAGCCACCGCGGTGGCGGCCTAGAGT | |
| | CGACGAGGAACTGAAAAACCAGAAAGTTAAC | |
| | TGGTAAGTTTAGTCTTTTTGTCTTTTATTTCAGG | |
| | TCCCGGATCCGGTGGTGGTGCAAATCAAAGAA | |
| | CTGCTCCTCAGTGGATGTTGCCTTTACTTCTAG | |
| | GCCTGTACGGAAGTGTTACTTCTGCTCTAAAA | |
| | GCTGCGGAATTGTACCCGCGGCCGATCCACCG | |
| | GTCCGGAATTACGCGTCTCGAGAGAATTCGCC | |
| | ACCATGGTGTCTTATTGGGATACTGGCGTGCTG | |
| | CTCTGTGCCCTCCTGAGTTGCCTGCTCCTGACT | |
| | GGTTCTTCTTCTGGGTCCGATACTGGGCGCCCC | |
| | TTCGTGGAGATGTACTCCGAGATCCCTGAAAT | |
| | CATTCACATGACTGAGGGTCGGGAACTGGTCA | |
| | TCCCATGCCGCGTGACCTCTCCCAACATTACTG | |
| | TGACCCTGAAGAAATTCCCTCTGGACACCCTC | |
| | ATCCCAGATGGGAAGAGGATCATTTGGGACTC | |
| | AAGAAAGGGTTTTATCATCAGCAACGCTACAT | |
| | ACAAGGAGATTGGCCTGCTCACCTGCGAAGCA | |
| | ACAGTGAACGGACACCTGTACAAGACTAATTA | |
| | TCTCACCCATAGACAGACAAACACTATCATTG | |
| | ATGTGGTCCTGTCACCAAGCCACGGCATCGAG | |
| | CTCAGCGTCGGTGAAAAGCTGGTGCTCAATTG | |
| | TACAGCCCGGACTGAGCTGAACGTGGGCATTG | |
| | ACTTCAATTGGGAATACCCCAGCTCCAAGCAC | |
| | CAGCATAAGAAACTGGTGAACCGCGATCTCAA | |
| | AACCCAGTCCGGATCTGAGATGAAGAAATTTC | |
| | TGAGCACCCTCACAATCGACGGCGTGACACGA | |
| | TCCGATCAGGGACTGTATACTTGCGCCGCTTCT | |
| | AGTGGCCTGATGACCAAGAAAAATAGCACATT | |
| | CGTCAGGGTGCACGAAAAGGGCGGCGGCGGA | |
| | GGCAGCGGCGGCGGCGGAGGCGCCCAGCAAG | |
| | AAGAGTGCGAGTGGGATCCCTGGACCTGCGA | |
| | GCACATGGGATCCGGCAGCGCCACCGGAGGAT | |
| | CCGGAAGCACCGCCTCCAGCGGCTCCGGCAG | |
| | CGCCACCCACCAGGAGGAGTGTGAGTGGGAC | |
| | CCCTGGACCTGCGAACACATGCTGGAGGACA | |
| | AAACTCATACCTGCCCACCTTGTCCAGCACCA | |
| | GAGCTGCTCGGAGGACCATCCGTGTTCCTGTT | |
| | TCCACCCAAGCCCAAAGATACTCTGATGATTTC | |
| | ACGCACACCCGAAGTCACTTGCGTGGTCGTGG | |
| | ACGTGTCCCACGAGGACCCCGAAGTCAAGTTT | |
| | AACTGGTACGTGGACGGCGTCGAGGTGCATAA | |
| | TGCTAAGACAAAACCCCGAGAGGAACAGTAC | |
| | AACTCTACCTATAGGGTCGTGAGTGTCCTGAC | |
| | AGTGCTCCACCAGGATTGGCTGAACGGAAAG | |
| | GAGTATAAGTGCAAAGTGTCTAATAAGGCACT | |
| | GCCTGCCCCAATCGAGAAAACAATTAGTAAGG | |
| | CCAAAGGGCAGCCCAGAGAACCTCAGGTGTA | |
| | CACTCTGCCTCCATCTCGGGACGAGCTCACTA | |
| | AGAACCAGGTCAGTCTGACCTGTCTCGTGAAA | |
| | GGGTTCTATCCTAGTGATATCGCTGTGGAGTGG | |
| | GAATCAAATGGTCAGCCAGAGAACAATTACAA | |
| | GACCACACCCCCTGTCCTGGACAGCGATGGCT | |
| | CCTTCTTTCTGTATTCCAAGCTCACCGTGGACA | |
| | AATCTCGATGGCAGCAGGGAAACGTCTTTAGT | |
| | TGTTCAGTGATGCACGAAGCCCTCCATAACCA | |
| | CTACACTCAGAAAAGCCTCAGCCTCAGCCCTG | |
| | GGAAATGATAAGCGGCCGCAAGCTTAAGAGAT | |
| | CTATAATCAACCTCTGGATTACAAAATTTGTGA | |
| | AAGATTGACTGGTATTCTTAACTATGTTGCTCC | |
| | TTTTACGCTATGTGGATACGCTGCTTTAATGCCT | |
| | TTGTATCATGCTATTGCTTCCCGTATGGCTTTCA | |
| | TTTTCTCCTCCTTGTATAAATCCTGGTTAGTTCT | |
| | TGCCACGGCGGAACTCATCGCCGCCTGCCTTG | |
| | CCCGCTGCTGGACAGGGGCTCGGCTGTTGGGC | |
| | ACTGACAATTCCGTGGTGTTAAGCTTATCGATA | |
| | CCGTCGACTAGAGCTCGCTGATCAGCCTCGAC | |
| | TGTGCCTTCTAGTTGCCAGCCATCTGTTGTTTG | |
| | CCCCTCCCCCGTGCCTTCCTTGACCCTGGAAG | |
| | GTGCCACTCCCACTGTCCTTTCCTAATAAAATG | |
| | AGGAAATTGCATCGCATTGTCTGAGTAGGTGT | |
| | CATTCTATTCTGGGGGGTGGGGTGGGGCAGGA | |
| | CAGCAAGGGGGAGGATTGGGAAGTCTAGAGC | |
| | AGGCATGCTGGGGAGAGATCGATCTGAGGAAC | |
| | CCCTAGTGATGGAGTTGGCCACTCCCTCTCTG | |
| | CGCGCTCGCTCGCTCACTGAGGCCGGGCGACC | |
| | AAAGGTCGCCCGACGCCCGGGCTTTGCCCGG | |
| | GCGGCCTCAGTGAGCGAGCGAGCGCGCAGAG | |
| | AGGGAGTGGCC | |
| EXG102-11 | TTGGCCACTCCCTCTCTGCGCGCTCGCTCGCTC | SEQ ID NO: 21 |
| | ACTGAGGCCGGGCGACCAAAGGTCGCCCGAC | |
| | GCCCGGGCTTTGCCCGGGCGGCCTCAGTGAGC | |
| | GAGCGAGCGCGCAGAGAGGGAGTGGCCAACT | |
| | CCATCACTAGGGGTTCCTCAGATCTGAATTCGG | |
| | TACCTAGTTATTAATAGTAATCAATTACGGGGTC | |
| | ATTAGTTCATAGCCCATATATGGAGTTCCGCGTT | |
| | ACATAACTTACGGTAAATGGCCCGCCTGGCTG | |
| | ACCGCCCAACGACCCCCGCCCATTGACGTCAA | |
| | TAATGACGTATGTTCCCATAGTAACGCCAATAG | |
| | GGACTTTCCATTGACGTCAATGGGTGGAGTATT | |
| | TACGGTAAACTGCCCACTTGGCAGTACATCAA | |
| | GTGTATCATATGCCAAGTACGCCCCCTATTGAC | |
| | GTCAATGACGGTAAATGGCCCGCCTGGCATTAT | |
| | GCCCAGTACATGACCTTATGGGACTTTCCTACT | |
| | TGGCAGTACATCTACGTATTAGTCATCGCTATTA | |
| | CCATGGTCGAGGTGAGCCCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | TCGCTGCGCGCTGCCTTCGCCCCGTGCCCCGC | |
| | TCCGCCGCCGCCTCGCGCCGCCCGCCCCGGCT | |
| | CTGACTGACCGCGTTACTCCCACAGGTGAGCG | |
| | GGCGGGACGGCCCTTCTCCTCCGGGCTGTAAT | |
| | TAGCGCTTGGTTTAATGACGGCTTGTTTCTTTT | |
| | CTGTGGCTGCGTGAAAGCCTTGAGGGGCTCCG | |
| | GGAGGGCCCTTTGTGCGGGGGGAGCGGCTCG | |
| | GGGGGTGCGTGCGTGTGTGTGTGCGTGGGGA | |
| | GCGCCGCGTGCGGCTCCGCGCTGCCCGGCGGC | |
| | TGTGAGCGCTGCGGGCGCGGCGCGGGGCTTT | |
| | GTGCGCTCCGCAGTGTGCGCGAGGGGAGCGC | |
| | GGCCGGGGGCGGTGCCCCGCGGTGCGGGGGG | |
| | GGCTGCGAGGGGAACAAAGGCTGCGTGCGGG | |
| | GTGTGTGCGTGGGGGGGTGAGCAGGGGGTGT | |
| | GGGCGCGTCGGTCGGGCTGCAACCCCCCCTGC | |
| | ACCCCCCTCCCCGAGTTGCTGAGCACGGCCCG | |
| | GCTTCGGGTGCGGGGCTCCGTACGGGGCGTGG | |
| | CGCGGGGCTCGCCGTGCCGGGCGGGGGGTGG | |
| | CGGCAGGTGGGGGTGCCGGGCGGGGCGGGGC | |
| | CGCCTCGGGCCGGGGAGGGCTCGGGGGAGGG | |
| | GCGCGGCGGCCCCCGGAGCGCCGGCGGCTGT | |
| | CGAGGCGCGGCGAGCCGCAGCCATTGCCTTTT | |
| | ATGGTAATCGTGCGAGAGGGCGCAGGGACTTC | |
| | CTTTGTCCCAAATCTGTGCGGAGCCGAAATCT | |
| | GGGAGGCGCCGCCGCACCCCCTCTAGCGGGC | |
| | GCGGGGCGAAGCGGTGCGGCGCCGGCAGGAA | |
| | GGAAATGGGCGGGGAGGGCCTTCGTGCGTCG | |
| | CCGCGCCGCCGTCCCCTTCTCCCTCTCCAGCCT | |
| | CGGGGCTGTCCGCGGGGGGACGGCTGCCTTC | |
| | GGGGGGGACGGGGCAGGGCGGGGTTCGGCTT | |
| | CTGGCGTGTGACCGGCGGCTCTAGAGCCTCTG | |
| | CTAACCATGTTCATGCCTTCTTCTTTTTCCTACA | |
| | GCTCCTGGGCAACGTGCTGGTTATTGTGCTGTC | |
| | TCATCATTTTGGCAAAGAATTCCTCGAAGATCT | |
| | AGGCAACGCGTCTCGAACGCGTCTCGAGAGA | |
| | ATTCGCCACCATGGTGTCTTATTGGGATACTGG | |
| | CGTGCTGCTCTGTGCCCTCCTGAGTTGCCTGCT | |
| | CCTGACTGGTTCTTCTTCTGGGGGCGGCGGCG | |
| | GAGGCGCCCAGCAAGAAGAGTGCGAGTGGGA | |
| | TCCCTGGACCTGCGAGCACATGGGATCCGGCA | |
| | GCGCCACCGGAGGATCCGGAAGCACCGCCTC | |
| | CAGCGGCTCCGGCAGCGCCACCCACCAGGAG | |
| | GAGTGTGAGTGGGACCCCTGGACCTGCGAAC | |
| | ACATGCTGGAGGGCGGCGGCGGAGGCAGCTC | |
| | CGATACTGGGCGCCCCTTCGTGGAGATGTACT | |
| | CCGAGATCCCTGAAATCATTCACATGACTGAG | |
| | GGTCGGGAACTGGTCATCCCATGCCGCGTGAC | |
| | CTCTCCCAACATTACTGTGACCCTGAAGAAAT | |
| | TCCCTCTGGACACCCTCATCCCAGATGGGAAG | |
| | AGGATCATTTGGGACTCAAGAAAGGGTTTTAT | |
| | CATCAGCAACGCTACATACAAGGAGATTGGCC | |
| | TGCTCACCTGCGAAGCAACAGTGAACGGACA | |
| | CCTGTACAAGACTAATTATCTCACCCATAGACA | |
| | GACAAACACTATCATTGATGTGGTCCTGTCACC | |
| | AAGCCACGGCATCGAGCTCAGCGTCGGTGAA | |
| | AAGCTGGTGCTCAATTGTACAGCCCGGACTGA | |
| | GCTGAACGTGGGCATTGACTTCAATTGGGAAT | |
| | ACCCCAGCTCCAAGCACCAGCATAAGAAACTG | |
| | GTGAACCGCGATCTCAAAACCCAGTCCGGATC | |
| | TGAGATGAAGAAATTTCTGAGCACCCTCACAA | |
| | TCGACGGCGTGACACGATCCGATCAGGGACTG | |
| | TATACTTGCGCCGCTTCTAGTGGCCTGATGACC | |
| | AAGAAAAATAGCACATTCGTCAGGGTGCACGA | |
| | AAAGGACAAAACTCATACCTGCCCACCTTGTC | |
| | CAGCACCAGAGCTGCTCGGAGGACCATCCGTG | |
| | TTCCTGTTTCCACCCAAGCCCAAAGATACTCT | |
| | GATGATTTCACGCACACCCGAAGTCACTTGCG | |
| | TGGTCGTGGACGTGTCCCACGAGGACCCCGA | |
| | AGTCAAGTTTAACTGGTACGTGGACGGCGTCG | |
| | AGGTGCATAATGCTAAGACAAAACCCCGAGAG | |
| | GAACAGTACAACTCTACCTATAGGGTCGTGAG | |
| | TGTCCTGACAGTGCTCCACCAGGATTGGCTGA | |
| | ACGGAAAGGAGTATAAGTGCAAAGTGTCTAAT | |
| | AAGGCACTGCCTGCCCCAATCGAGAAAACAAT | |
| | TAGTAAGGCCAAAGGGCAGCCCAGAGAACCT | |
| | CAGGTGTACACTCTGCCTCCATCTCGGGACGA | |
| | GCTCACTAAGAACCAGGTCAGTCTGACCTGTC | |
| | TCGTGAAAGGGTTCTATCCTAGTGATATCGCTG | |
| | TGGAGTGGGAATCAAATGGTCAGCCAGAGAA | |
| | CAATTACAAGACCACACCCCCTGTCCTGGACA | |
| | GCGATGGCTCCTTCTTTCTGTATTCCAAGCTCA | |
| | CCGTGGACAAATCTCGATGGCAGCAGGGAAA | |
| | CGTCTTTAGTTGTTCAGTGATGCACGAAGCCCT | |
| | CCATAACCACTACACTCAGAAAAGCCTCAGCC | |
| | TCAGCCCTGGGAAATGATAAGCGGCCGCAAGC | |
| | TTAAGAGCTTAAGCTAGAGCTCGCTGATCAGC | |
| | CTCGACTGTGCCTTCTAGTTGCCAGCCATCTGT | |
| | TGTTTGCCCCTCCCCCGTGCCTTCCTTGACCCT | |
| | GGAAGGTGCCACTCCCACTGTCCTTTCCTAATA | |
| | AAATGAGGAAATTGCATCGCATTGTCTGAGTA | |
| | GGTGTCATTCTATTCTGGGGGGTGGGGTGGGG | |
| | CAGGACAGCAAGGGGGAGGATTGGGAAGACA | |
| | ATAGCAGGCATGCTGGGGAGAGTCTAGAGTCG | |
| | ACTGGGGAGAGATCTGAGGAACCCCTAGTGAT | |
| | GGAGTTGGCCACTCCCTCTCTGCGCGCTCGCT | |
| | CGCTCACTGAGGCCGCCCGGGCAAAGCCCGG | |
| | GCGTCGGGCGACCTTTGGTCGCCCGGCCTCAG | |
| | TGAGCGAGCGAGCGCGCAGAGAGGGAGTGGC | |
| | CAAC | |
| EXG102-12 | CTGCGCGCTCGCTCGCTCACTGAGGCCGCCCG | SEQ ID NO: 22 |
| | GGCAAAGCCCGGGCGTCGGGCGACCTTTGGT | |
| | CGCCCGGCCTCAGTGAGCGAGCGAGCGCGCA | |
| | GAGAGGGAGTGGAATGCACGCGTGGATCTGA | |
| | GTTCAATTCACGCGTGGTACCTCTGGTCGTTAC | |
| | ATAACTTACGGTAAATGGCCCGCCTGGCTGAC | |
| | CGCCCAACGACCCCCGCCCATTGACGTCAATA | |
| | ATGACGTATGTTCCCATAGTAACGCCAATAGGG | |
| | ACTTTCCATTGACGTCAATGGGTGGAGTATTTA | |
| | CGGTAAACTGCCCACTTGGCAGTACATCAAGT | |
| | GTATCATATGCCAAGTACGCCCCCTATTGACGT | |
| | CAATGACGGTAAATGGCCCGCCTGGCATTATGC | |
| | CCAGTACATGACCTTATGGGACTTTCCTACTTG | |
| | GCAGTACATCTACTCGAGGCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | CGGGATCAGCCACCGCGGTGGCGGCCTAGAGT | |
| | CGACGAGGAACTGAAAAACCAGAAAGTTAAC | |
| | TGGTAAGTTTAGTCTTTTTGTCTTTTATTTCAGG | |
| | TCCCGGATCCGGTGGTGGTGCAAATCAAAGAA | |
| | CTGCTCCTCAGTGGATGTTGCCTTTACTTCTAG | |
| | GCCTGTACGGAAGTGTTACTTCTGCTCTAAAA | |
| | GCTGCGGAATTGTACCCGCGGCCGATCCACCG | |
| | GTCCGGAATTCGCCACCATGGTGTCTTATTGGG | |
| | ATACTGGCGTGCTGCTCTGTGCCCTCCTGAGTT | |
| | GCCTGCTCCTGACTGGTTCTTCTTCTGGGGGC | |
| | GGCGGCGGAGGCGCCCAGCAAGAAGAGTGCG | |
| | AGTGGGATCCCTGGACCTGCGAGCACATGGGA | |
| | TCCGGCAGCGCCACCGGAGGATCCGGAAGCA | |
| | CCGCCTCCAGCGGCTCCGGCAGCGCCACCCAC | |
| | CAGGAGGAGTGTGAGTGGGACCCCTGGACCT | |
| | GCGAACACATGCTGGAGGGCGGCGGCGGAGG | |
| | CAGCTCCGATACTGGGCGCCCCTTCGTGGAGA | |
| | TGTACTCCGAGATCCCTGAAATCATTCACATGA | |
| | CTGAGGGTCGGGAACTGGTCATCCCATGCCGC | |
| | GTGACCTCTCCCAACATTACTGTGACCCTGAA | |
| | GAAATTCCCTCTGGACACCCTCATCCCAGATG | |
| | GGAAGAGGATCATTTGGGACTCAAGAAAGGG | |
| | TTTTATCATCAGCAACGCTACATACAAGGAGAT | |
| | TGGCCTGCTCACCTGCGAAGCAACAGTGAAC | |
| | GGACACCTGTACAAGACTAATTATCTCACCCAT | |
| | AGACAGACAAACACTATCATTGATGTGGTCCT | |
| | GTCACCAAGCCACGGCATCGAGCTCAGCGTCG | |
| | GTGAAAAGCTGGTGCTCAATTGTACAGCCCGG | |
| | ACTGAGCTGAACGTGGGCATTGACTTCAATTG | |
| | GGAATACCCCAGCTCCAAGCACCAGCATAAGA | |
| | AACTGGTGAACCGCGATCTCAAAACCCAGTCC | |
| | GGATCTGAGATGAAGAAATTTCTGAGCACCCT | |
| | CACAATCGACGGCGTGACACGATCCGATCAGG | |
| | GACTGTATACTTGCGCCGCTTCTAGTGGCCTGA | |
| | TGACCAAGAAAAATAGCACATTCGTCAGGGTG | |
| | CACGAAAAGGACAAAACTCATACCTGCCCACC | |
| | TTGTCCAGCACCAGAGCTGCTCGGAGGACCAT | |
| | CCGTGTTCCTGTTTCCACCCAAGCCCAAAGAT | |
| | ACTCTGATGATTTCACGCACACCCGAAGTCAC | |
| | TTGCGTGGTCGTGGACGTGTCCCACGAGGACC | |
| | CCGAAGTCAAGTTTAACTGGTACGTGGACGGC | |
| | GTCGAGGTGCATAATGCTAAGACAAAACCCCG | |
| | AGAGGAACAGTACAACTCTACCTATAGGGTCG | |
| | TGAGTGTCCTGACAGTGCTCCACCAGGATTGG | |
| | CTGAACGGAAAGGAGTATAAGTGCAAAGTGTC | |
| | TAATAAGGCACTGCCTGCCCCAATCGAGAAAA | |
| | CAATTAGTAAGGCCAAAGGGCAGCCCAGAGA | |
| | ACCTCAGGTGTACACTCTGCCTCCATCTCGGG | |
| | ACGAGCTCACTAAGAACCAGGTCAGTCTGACC | |
| | TGTCTCGTGAAAGGGTTCTATCCTAGTGATATC | |
| | GCTGTGGAGTGGGAATCAAATGGTCAGCCAGA | |
| | GAACAATTACAAGACCACACCCCCTGTCCTGG | |
| | ACAGCGATGGCTCCTTCTTTCTGTATTCCAAGC | |
| | TCACCGTGGACAAATCTCGATGGCAGCAGGGA | |
| | AACGTCTTTAGTTGTTCAGTGATGCACGAAGC | |
| | CCTCCATAACCACTACACTCAGAAAAGCCTCA | |
| | GCCTCAGCCCTGGGAAATGATAAGCGGCCGCA | |
| | AGCTTATCGATACCGTCGACTAGAGCTCGCTG | |
| | ATCAGCCTCGACTGTGCCTTCTAGTTGCCAGC | |
| | CATCTGTTGTTTGCCCCTCCCCCGTGCCTTCCT | |
| | TGACCCTGGAAGGTGCCACTCCCACTGTCCTT | |
| | TCCTAATAAAATGAGGAAATTGCATCGCATTGT | |
| | CTGAGTAGGTGTCATTCTATTCTGGGGGGTGG | |
| | GGTGGGGCAGGACAGCAAGGGGGAGGATTGG | |
| | GAAGTCTAGAGCAGGCATGCTGGGGAGAGAT | |
| | CGATCTGAGGAACCCCTAGTGATGGAGTTGGC | |
| | CACTCCCTCTCTGCGCGCTCGCTCGCTCACTG | |
| | AGGCCGGGCGACCAAAGGTCGCCCGACGCCC | |
| | GGGCTTTGCCCGGGCGGCCTCAGTGAGCGAG | |
| | CGAGCGCGCAGAGAGGGAGTGGCC | |
| EXG102-13 | CTGCGCGCTCGCTCGCTCACTGAGGCCGCCCG | SEQ ID NO: 23 |
| | GGCAAAGCCCGGGCGTCGGGCGACCTTTGGT | |
| | CGCCCGGCCTCAGTGAGCGAGCGAGCGCGCA | |
| | GAGAGGGAGTGGAATGCACGCGTGGATCTGA | |
| | GTTCAATTCACGCGTGGTACCTCTGGTCGTTAC | |
| | ATAACTTACGGTAAATGGCCCGCCTGGCTGAC | |
| | CGCCCAACGACCCCCGCCCATTGACGTCAATA | |
| | ATGACGTATGTTCCCATAGTAACGCCAATAGGG | |
| | ACTTTCCATTGACGTCAATGGGTGGAGTATTTA | |
| | CGGTAAACTGCCCACTTGGCAGTACATCAAGT | |
| | GTATCATATGCCAAGTACGCCCCCTATTGACGT | |
| | CAATGACGGTAAATGGCCCGCCTGGCATTATGC | |
| | CCAGTACATGACCTTATGGGACTTTCCTACTTG | |
| | GCAGTACATCTACTCGAGGCCACGTTCTGCTTC | |
| | ACTCTCCCCATCTCCCCCCCCTCCCCACCCCCA | |
| | ATTTTGTATTTATTTATTTTTTAATTATTTTGTGC | |
| | AGCGATGGGGGCGGGGGGGGGGGGGGGGCGC | |
| | GCGCCAGGCGGGGCGGGGCGGGGCGAGGGGC | |
| | GGGGCGGGGCGAGGCGGAGAGGTGCGGCGGC | |
| | AGCCAATCAGAGCGGCGCGCTCCGAAAGTTTC | |
| | CTTTTATGGCGAGGCGGCGGCGGCGGCGGCCC | |
| | TATAAAAAGCGAAGCGCGCGGCGGGCGGGAG | |
| | CGGGATCAGCCACCGCGGTGGCGGCCTAGAGT | |
| | CGACGAGGAACTGAAAAACCAGAAAGTTAAC | |
| | TGGTAAGTTTAGTCTTTTTGTCTTTTATTTCAGG | |
| | TCCCGGATCCGGTGGTGGTGCAAATCAAAGAA | |
| | CTGCTCCTCAGTGGATGTTGCCTTTACTTCTAG | |
| | GCCTGTACGGAAGTGTTACTTCTGCTCTAAAA | |
| | GCTGCGGAATTGTACCCGCGGCCGATCCACCG | |
| | GTCCGGAATTACGCGTCTCGAGAGAATTCGCC | |
| | ACCATGGTGTCTTATTGGGATACTGGCGTGCTG | |
| | CTCTGTGCCCTCCTGAGTTGCCTGCTCCTGACT | |
| | GGTTCTTCTTCTGGGTCCGATACTGGGCGCCCC | |
| | TTCGTGGAGATGTACTCCGAGATCCCTGAAAT | |
| | CATTCACATGACTGAGGGTCGGGAACTGGTCA | |
| | TCCCATGCCGCGTGACCTCTCCCAACATTACTG | |
| | TGACCCTGAAGAAATTCCCTCTGGACACCCTC | |
| | ATCCCAGATGGGAAGAGGATCATTTGGGACTC | |
| | AAGAAAGGGTTTTATCATCAGCAACGCTACAT | |
| | ACAAGGAGATTGGCCTGCTCACCTGCGAAGCA | |
| | ACAGTGAACGGACACCTGTACAAGACTAATTA | |
| | TCTCACCCATAGACAGACAAACACTATCATTG | |
| | ATGTGGTCCTGTCACCAAGCCACGGCATCGAG | |
| | CTCAGCGTCGGTGAAAAGCTGGTGCTCAATTG | |
| | TACAGCCCGGACTGAGCTGAACGTGGGCATTG | |
| | ACTTCAATTGGGAATACCCCAGCTCCAAGCAC | |
| | CAGCATAAGAAACTGGTGAACCGCGATCTCAA | |
| | AACCCAGTCCGGATCTGAGATGAAGAAATTTC | |
| | TGAGCACCCTCACAATCGACGGCGTGACACGA | |
| | TCCGATCAGGGACTGTATACTTGCGCCGCTTCT | |
| | AGTGGCCTGATGACCAAGAAAAATAGCACATT | |
| | CGTCAGGGTGCACGAAAAGGACAAAACTCAT | |
| | ACCTGCCCACCTTGTCCAGCACCAGAGCTGCT | |
| | CGGAGGACCATCCGTGGGCGGCGGCGGAGGC | |
| | AGCGGCGGCGGCGGAGGCGCCCAGCAAGAAG | |
| | AGTGCGAGTGGGATCCCTGGACCTGCGAGCAC | |
| | ATGGGATCCGGCAGCGCCACCGGAGGATCCGG | |
| | AAGCACCGCCTCCAGCGGCTCCGGCAGCGCC | |
| | ACCCACCAGGAGGAGTGTGAGTGGGACCCCT | |
| | GGACCTGCGAACACATGCTGGAGTGATAAGCG | |
| | GCCGCAAGCTTAAGAGATCTATAATCAACCTCT | |
| | GGATTACAAAATTTGTGAAAGATTGACTGGTAT | |
| | TCTTAACTATGTTGCTCCTTTTACGCTATGTGGA | |
| | TACGCTGCTTTAATGCCTTTGTATCATGCTATTG | |
| | CTTCCCGTATGGCTTTCATTTTCTCCTCCTTGTA | |
| | TAAATCCTGGTTAGTTCTTGCCACGGCGGAAC | |
| | TCATCGCCGCCTGCCTTGCCCGCTGCTGGACA | |
| | GGGGCTCGGCTGTTGGGCACTGACAATTCCGT | |
| | GGTGTTAAGCTTATCGATACCGTCGACTAGAGC | |
| | TCGCTGATCAGCCTCGACTGTGCCTTCTAGTTG | |
| | CCAGCCATCTGTTGTTTGCCCCTCCCCCGTGCC | |
| | TTCCTTGACCCTGGAAGGTGCCACTCCCACTG | |
| | TCCTTTCCTAATAAAATGAGGAAATTGCATCGC | |
| | ATTGTCTGAGTAGGTGTCATTCTATTCTGGGGG | |
| | GTGGGGTGGGGCAGGACAGCAAGGGGGAGG | |
| | ATTGGGAAGTCTAGAGCAGGCATGCTGGGGAG | |
| | AGATCGATCTGAGGAACCCCTAGTGATGGAGT | |
| | TGGCCACTCCCTCTCTGCGCGCTCGCTCGCTC | |
| | ACTGAGGCCGGGCGACCAAAGGTCGCCCGAC | |
| | GCCCGGGCTTTGCCCGGGCGGCCTCAGTGAGC | |
| | GAGCGAGCGCGCAGAGAGGGAGTGGCC | |

### 6.3. Example 3—Protein Expressions and Ligand Binding Assays

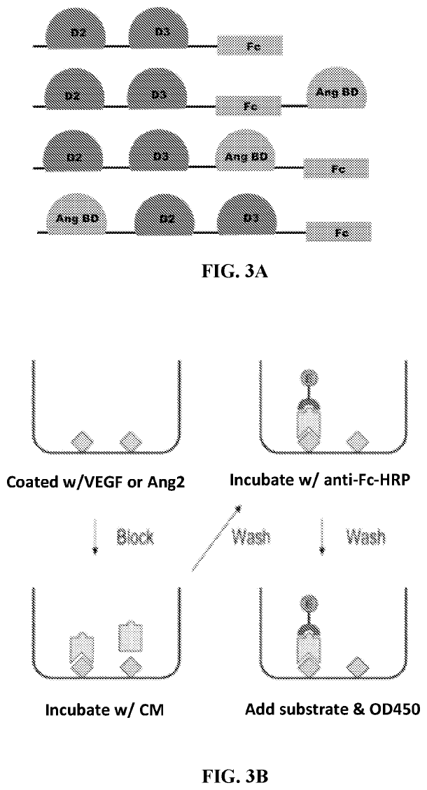

HEK293 cells were transiently transfected with plasmid constructs or infected with AAV vectors expressing D2D3 or D2D3/ABD (see FIG. 3A). Cultured medium containing D2D3 or D2D3/ABD were added to the wells coated with VEGF or Ang that were capable of capturing D2D3 or ABD, respectively. Captured D2D3 or D2D3/ABD were then incubated with anti-Fc-HRP and quantitated by reading OD450 (see FIG. 3B).

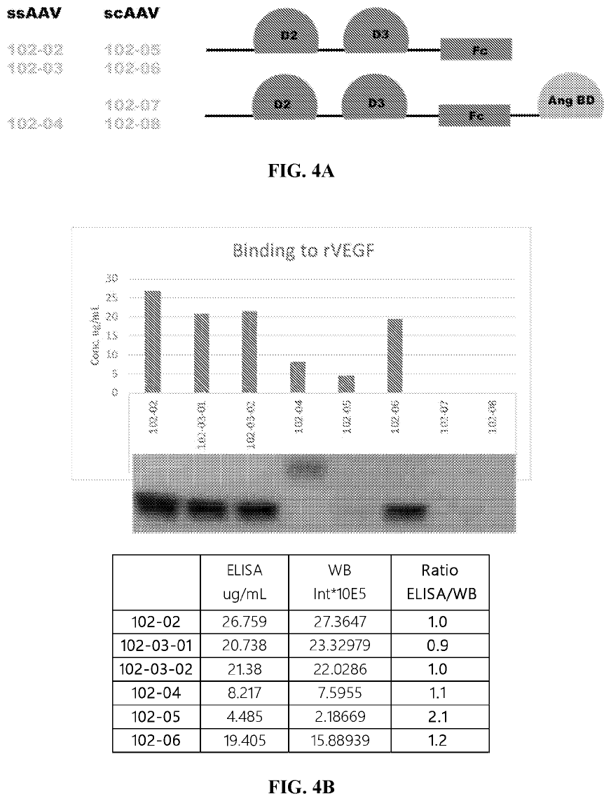

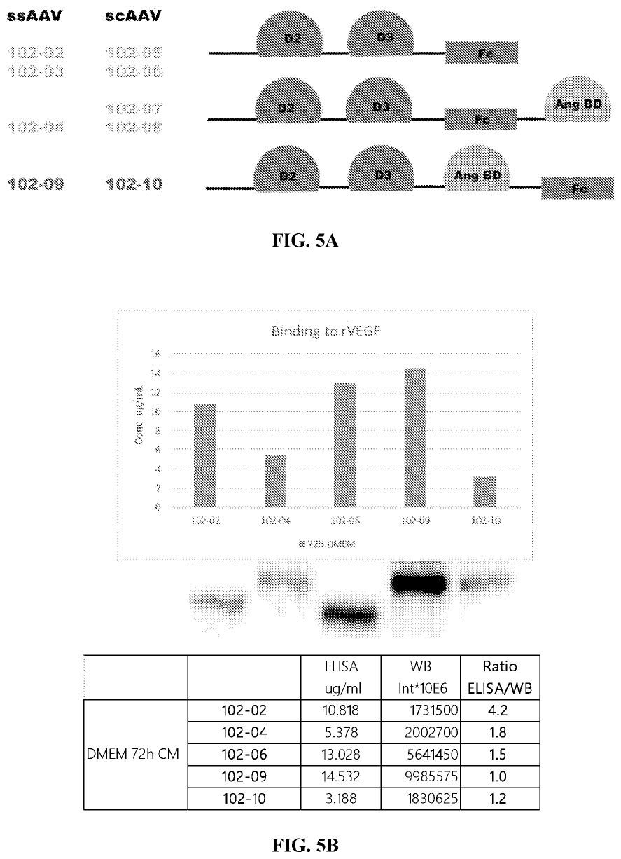

The results show that transgene expression from 3 codon optimized version EXG102-02, EXG102-03-01, and EXG102-03-02 were comparable with scAAV102-06. Position of Ang BD in C-terminus did not affect the binding ability of D2D3 to rVEGF, however it severely reduced the transgene expression levels (see EXG102-04, EXG102-07 and EXG102-08) as shown in FIGS. 4A-4B and 5A-5B.

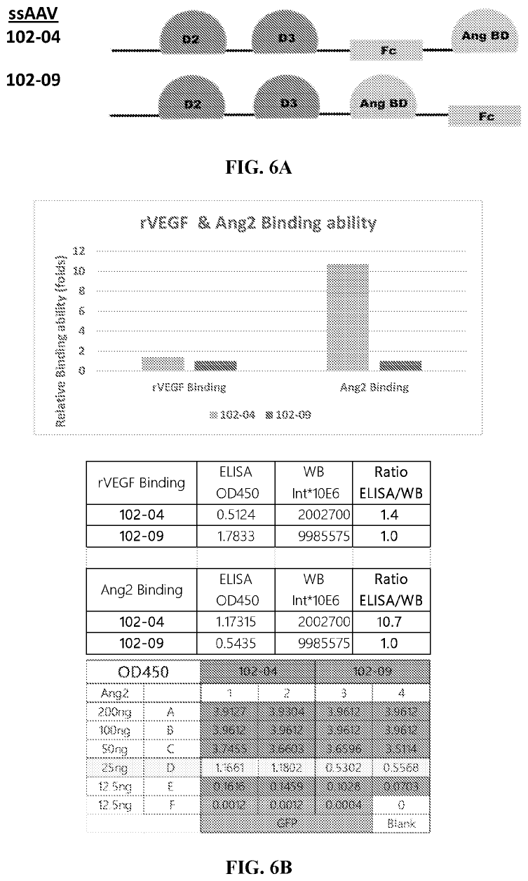

In contrast, position of Ang BD in the middle between D2D3 and Fc did not affect the binding ability to rVEGF and transgene expression (see EXG102-09) as shown in FIGS. 5A-5B. However, position of Ang BD in the middle inhibited the binding ability to Ang2 as shown in FIGS. 6A-6B.

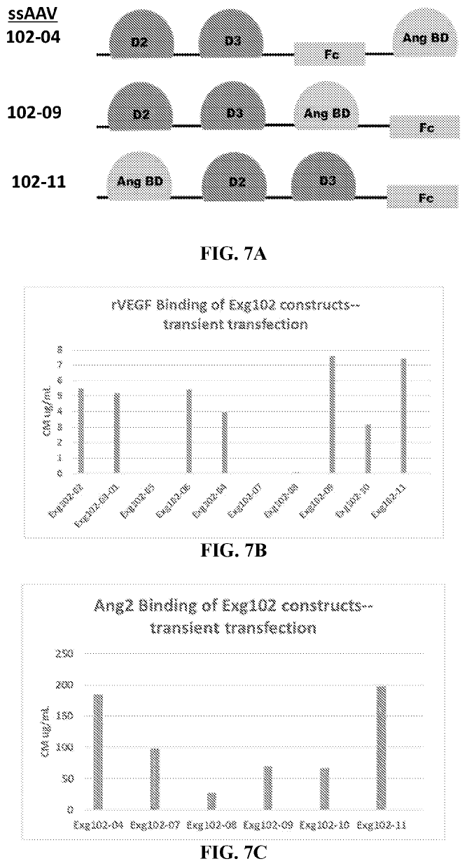

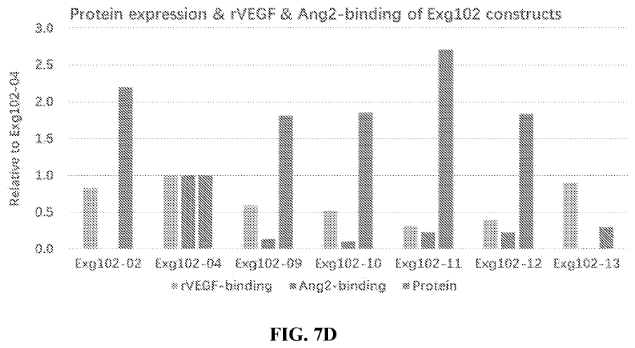

As shown in FIGS. 7A-7C, the constructs that have Ang BD at the N-terminus (e.g., EXG102-11) could bind VEGF as well as construct EXG102-09 with equal CM from transient transfection; in addition, EXG102-04 and EXG102-11 showed strongest binding to Ang2 with equal CM from transient transfection. In addition, EXG102-11 did not reduce transgene expression (see FIG. 7D). Taken together, EXG102-11 was shown to bind strongly to both VEGF and Ang2, while maintaining high transgene expression level.

### 6.4. Example 4—Construction of Fusion Proteins Comprising an Additional VEGFC Binding Domain (Trap C)

Additional exemplary fusion proteins according to the present disclosure were constructed as shown in FIG. 8. These additional constructs comprise an additional VEGF binding domain, i.e., a VEGFC binding domain (Trap C). FIGS. 9A-9I illustrate exemplary polypeptide constructs provided herein as encoded by the nucleic acid (transgene) in EXG102-24, EXG102-25, EXG102-26, EXG102-27, EXG102-28, and EXG102-29, respectively. Signal peptide, ABD, D2, D3, Trap C, and Fc constructs are indicated in these figures. Specifically, these exemplary fusion proteins comprise four binding domains-one derived from VEGFR-1 (i.e., the IgG-like domain 2 of VEGFR-1), one derived from VEGFR-2 (i.e., the IgG like domain 3 of VEGFR-2), one VEGFC binding domain, and one domain capable of binding to an angiopoietin (e.g., angiopoietin 1 and angiopoietin 2). These fusion proteins further comprise an Fc fragment of human IgG. An Flt-1 signal domain was also included at the N-terminus in these exemplary constructs.

In some of the present constructs, the ABD comprises one or more repeat(s) of amino acid sequence having SEQ ID NO: 3 or SEQ ID NO: 51. In some constructs, the ABD provided herein comprises one repeat of SEQ ID NO: 3. In some constructs, the ABD provided herein comprises two repeats of SEQ ID NO: 3. In some constructs, the ABD provided herein comprises one repeat of SEQ ID NO: 51. In some constructs, the ABD provided herein comprises two repeats of SEQ ID NO: 51. In some constructs, the ABD provided herein comprises SEQ ID NO: 4. In some constructs, the ABD provided herein comprises SEQ ID NO: 52. In some constructs, the ABD provided herein comprises SEQ ID NO: 53. In some constructs, the ABD provided herein comprises SEQ ID NO: 54.

Exemplary Trap C sequences that can included in the present constructs are provided below. In some constructs, the Trap C comprises an amino acid sequence of SEQ ID NO: 55. In some constructs, the Trap C comprises an amino acid sequence of SEQ ID NO: 56. In some constructs, the Trap C comprises an amino acid sequence of SEQ ID NO: 57.

These sequences for these exemplary domains and exemplary fusion proteins are provided in the table below.

TABLE 6
Sequences for Exemplary Domains and Exemplary Fusion Proteins
Comprising a Trap C

| Region | Sequence | SEQ ID NO |
|---|---|---|
| Exemplary first | SDTGRPFVEMYSEIPEIIHMTEGRELVIPCRVTSP | SEQ ID NO: 1 |
| domain derived | NITVTLKKFPLDTLIPDGKRIIWDSRKGFIISNATY | |
| from VEGFR-1 | KEIGLLTCEATVNGHLYKTNYLTHRQTNTIID | |
| (D2) | | |
| Exemplary | VVLSPSHGIELSVGEKLVLNCTARTELNVGIDEN | SEQ ID NO: 2 |
| second domain | WEYPSSKHQHKKLVNRDLKTQSGSEMKKFLST | |
| derived from | LTIDGVTRSDQGLYTCAASSGLMTKKNSTFVRV | |
| VEGFR-2 (D3) | HE | |
| 14 amino acid | QEECEWDPWTCEHM | SEQ ID NO: 3 |
| sequence in | | |
| exemplary ABD | | |
| (Con4) | | |
| Exemplary | GGGGGAQQEECEWDPWTCEHMGSGSATGGSG | SEQ ID NO: 4 |
| ABD (1) | STASSGSGSATHQEECEWDPWTCEHMLE | |
| (2xCon4) | | |
| L1 | KFNPLDELEETLYEQFTFQQ | SEQ ID NO: 51 |
| Exemlary | GGGGGAQKFNPLDELEETLYEQFTFQQGGGGG | SEQ ID NO: 52 |
| ABD (2) (2xL1) | GGGKFNPLDELEETLYEQFTFQQLE | |
| Exemplary | GGGGGAQQEECEWDPWTCEHMGGGGGGGGK | SEQ ID NO: 53 |
| ABD (3) (Con4- | FNPLDELEETLYEQFTFQQLE | |
| L1) | | |
| Exemplary | GGGGGAQGSGSATGGSGSTASSGSGSATHKFNP | SEQ ID NO: 54 |
| ABD (4) (L1- | LDELEETLYEQFTFQQGGGGGQEECEWDPWTC | |
| Con4) | EHMLE | |
| Exemplary | VYVQDYRSPFIASVSDQHGVVYITENKNKTVVI | SEQ ID NO: 55 |
| Trap C (1) | PCLGSISNLNVSLCARYPEKRFVPDGNRISWDSK | |
| derived from | KGFTIPSYMISYAGMVFCEAKINDESYQSIMYIV | |
| VEGFR-2 | VVVG | |
| Exemplary | YSMTPPTLNITEESHVIDTGDSLSISCRGQHPLEW | SEQ ID NO: 56 |
| Trap C (2) | AWPGAQEAPATGDKDSEDTGVVRDCEGTDARP | |
| derived from | YCKVLLLHEVHAQDTGSYVCYYKYIKARIEGTT | |
| VEGFR-3 | AASSYVFVRDFEQPFINKPDTLLVNRKDAMWVP | |
| | CLVSIPGLNVTLRSQSSVLWPDGQEVVWDDRRG | |
| | MLVSTPLLHDALYLQCETTWGDQDFLSNPFLVH | |
| | ITG | |
| Exemplary | YSMTPPTLNITEESHVIDTGDSLSISCRGQHPLEW | SEQ ID NO: 57 |
| Trap C (3) | AWPGAQEAPATGDKDSEDTGVVRDCEGTDARP | |
| derived from | YCKVLLLHEVHANDTGSYVCYYKYIKARIEGTT | |
| VEGFR-3 | AASSYVFVRDFEQPFINKPDTLLVNRKDAMWVP | |
| | CLVSIPGLNVTLRSQSSVLWPDGQEVVWDDRRG | |
| | MLVSTPLLHDALYLQCETTWGDQDFLSNPFLVH | |
| | ITG | |
| Exemplary IgG- | DKTHTCPPCPAPELLGGPSVFLFPPKPKDTLMISR | SEQ ID NO: 5 |
| Fc (1) | TPEVTCVVVDVSHEDPEVKFNWYVDGVEVHNA | |
| | KTKPREEQYNSTYRVVSVLTVLHQDWLNGKEY | |
| | KCKVSNKALPAPIEKTISKAKGQPREPQVYTLPP | |
| | SRDELTKNQVSLTCLVKGFYPSDIAVEWESNGQP | |
| | ENNYKTTPPVLDSDGSFFLYSKLTVDKSRWQQG | |
| | NVFSCSVMHEALHNHYTQKSLSLSPGK | |
| Exemplary IgG- | KDKTHTCPPCPAPELLGGPSVFLFPPKPKDTLMIS | SEQ ID NO: 50 |
| Fc (2) | RTPEVTCVVVDVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYTLP | |
| | PSRDELTKNQVSLTCLVKGFYPSDIAVEWESNGQ | |
| | PENNYKTTPPVLDSDGSFFLYSKLTVDKSRWQQ | |
| | GNVFSCSVMHEALHNHYTQKSLSLSPGK | |
| Flt-1 Signal | MVSYWDTGVLLCALLSCLLLTGSSSG | SEQ ID NO: 6 |
| Exemplary | MVSYWDTGVLLCALLSCLLLTGSSSGGGGGGA | SEQ ID NO: 58 |
| Polypeptide 5 (as | QQEECEWDPWTCEHMGSGSATGGSGSTASSGS | |
| in EXG102-24) | GSATHQEECEWDPWTCEHMLEGGGGGSSDTGR | |
| | PFVEMYSEIPEIIHMTEGRELVIPCRVTSPNITVTL | |
| | KKFPLDTLIPDGKRIIWDSRKGFIISNATYKEIGL | |
| | LTCEATVNGHLYKTNYLTHRQTNTIIDVVLSPSH | |
| | GIELSVGEKLVLNCTARTELNVGIDENWEYPSSK | |
| | HQHKKLVNRDLKTQSGSEMKKFLSTLTIDGVTR | |
| | SDQGLYTCAASSGLMTKKNSTFVRVHEKGGGG | |
| | GSVYVQDYRSPFIASVSDQHGVVYITENKNKTV | |
| | VIPCLGSISNLNVSLCARYPEKRFVPDGNRISWD | |
| | SKKGFTIPSYMISYAGMVFCEAKINDESYQSIMY | |
| | IVVVVGDKTHTCPPCPAPELLGGPSVFLFPPKPK | |
| | DTLMISRTPEVTCVVVDVSHEDPEVKFNWYVD | |
| | GVEVHNAKTKPREEQYNSTYRVVSVLTVLHQD | |
| | WLNGKEYKCKVSNKALPAPIEKTISKAKGQPRE | |
| | PQVYTLPPSRDELTKNQVSLTCLVKGFYPSDIAV | |
| | EWESNGQPENNYKTTPPVLDSDGSFFLYSKLTV | |
| | DKSRWQQGNVFSCSVMHEALHNHYTQKSLSLS | |
| | PGK | |
| Exemplary | MVSYWDTGVLLCALLSCLLLTGSSSGSDTGRPF | SEQ ID NO: 59 |
| Polypeptide 6 (as | VEMYSEIPEIIHMTEGRELVIPCRVTSPNITVTLK | |
| in EXG102-25) | KFPLDTLIPDGKRIIWDSRKGFIISNATYKEIGLLT | |
| | CEATVNGHLYKTNYLTHRQTNTIIDVVLSPSHGI | |
| | ELSVGEKLVLNCTARTELNVGIDENWEYPSSKH | |
| | QHKKLVNRDLKTQSGSEMKKFLSTLTIDGVTRS | |
| | DQGLYTCAASSGLMTKKNSTFVRVHEKGGGGG | |
| | SVYVQDYRSPFIASVSDQHGVVYITENKNKTVV | |
| | IPCLGSISNLNVSLCARYPEKRFVPDGNRISWDS | |
| | KKGFTIPSYMISYAGMVFCEAKINDESYQSIMYI | |
| | VVVVGDKTHTCPPCPAPELLGGPSVFLFPPKPKD | |
| | TLMISRTPEVTCVVVDVSHEDPEVKFNWYVDG | |
| | VEVHNAKTKPREEQYNSTYRVVSVLTV | |
| | LHQDWLNGKEYKCKVSNKALPAPIEKTISKAKG | |
| | QPREPQVYTLPPSRDELTKNQVSLTCLVKGFYPS | |
| | DIAVEWESNGQPENNYKTTPPVLDSDGSFFLYS | |
| | KLTVDKSRWQQGNVFSCSVMHEALHNHYTQK | |
| | SLSLSPGKGGGGGAQQEECEWDPWTCEHMGSG | |
| | SATGGSGSTASSGSGSATHQEECEWDPWTCEH | |
| | MLE | |
| Exemplary | MVSYWDTGVLLCALLSCLLLTGSSSGSDTGRPF | SEQ ID NO: 60 |
| Polypeptide 7 (as | VEMYSEIPEIIHMTEGRELVIPCRVTSPNITVTLK | |
| in EXG102-26) | KFPLDTLIPDGKRIIWDSRKGFIISNATYKEIGLLT | |
| | CEATVNGHLYKTNYLTHRQTNTIIDVVLSPSHGI | |
| | ELSVGEKLVLNCTARTELNVGIDENWEYPSSKH | |
| | QHKKLVNRDLKTQSGSEMKKFLSTLTIDGVTRS | |
| | DQGLYTCAASSGLMTKKNSTFVRVHEKGGGGG | |
| | SVYVQDYRSPFIASVSDQHGVVYITENKNKTVV | |
| | IPCLGSISNLNVSLCARYPEKRFVPDGNRISWDS | |
| | KKGFTIPSYMISYAGMVFCEAKINDESYQSIMYI | |
| | VVVVGDKTHTCPPCPAPELLGGPSVFLFPPKPKD | |
| | TLMISRTPEVTCVVVDVSHEDPEVKFNWYVDG | |
| | VEVHNAKTKPREEQYNSTYRVVSVLTVLHQDW | |
| | LNGKEYKCKVSNKALPAPIEKTISKAKGQPREP | |
| | QVYTLPPSRDELTKNQVSLTCLVKGFYPSDIAVE | |
| | WESNGQPENNYKTTPPVLDSDGSFFLYSKLTVD | |
| | KSRWQQGNVFSCSVMHEALHNHYTQKSLSLSP | |
| | GKGGGGGAQGSGSATGGSGSTASSGSGSATHK | |
| | FNPLDELEETLYEQFTFQQGGGGGQEECEWDP | |
| | WTCEHMLE | |
| Exemplary | MVSYWDTGVLLCALLSCLLLTGSSSGGGGGGA | SEQ ID NO: 61 |
| Polypeptide 8 (as | QQEECEWDPWTCEHMGSGSATGGSGSTASSGS | |
| in EXG102-27) | GSATHQEECEWDPWTCEHMLEGGGGGSSDTGR | |
| | PFVEMYSEIPEIIHMTEGRELVIPCRVTSPNITVTL | |
| | KKFPLDTLIPDGKRIIWDSRKGFIISNATYKEIGL | |
| | LTCEATVNGHLYKTNYLTHRQTNTIIDVVLSPSH | |
| | GIELSVGEKLVLNCTARTELNVGIDENWEYPSSK | |
| | HQHKKLVNRDLKTQSGSEMKKFLSTLTIDGVTR | |
| | SDQGLYTCAASSGLMTKKNSTFVRVHEKGGGG | |
| | GSYSMTPPTLNITEESHVIDTGDSLSISCRGQHPL | |
| | EWAWPGAQEAPATGDKDSEDTGVVRDCEGTD | |
| | ARPYCKVLLLHEVHAQDTGSYVCYYKYIKARIE | |
| | GTTAASSYVFVRDFEQPFINKPDTLLVNRKDAM | |
| | WVPCLVSIPGLNVTLRSQSSVLWPDGQEVVWD | |
| | DRRGMLVSTPLLHDALYLQCETTWGDQDFLSN | |
| | PFLVHITGDKTHTCPPCPAPELLGGPSVFLFPPK | |
| | PKDTLMISRTPEVTCVVVDVSHEDPEVKFNWYV | |
| | DGVEVHNAKTKPREEQYNSTYRVVSVLTVLHQ | |
| | DWLNGKEYKCKVSNKALPAPIEKTISKAKGQPR | |
| | EPQVYTLPPSRDELTKNQVSLTCLVKGFYPSDIA | |
| | VEWESNGQPENNYKTTPPVLDSDGSFFLYSKLT | |
| | VDKSRWQQGNVFSCSVMHEALHNHYTQKSLSL | |
| | SPGK | |
| Exemplary | MVSYWDTGVLLCALLSCLLLTGSSSGSDTGRPF | SEQ ID NO: 62 |
| polypeptide 9 (as | VEMYSEIPEIIHMTEGRELVIPCRVTSPNITVTLK | |
| in EXG102-28) | KFPLDTLIPDGKRIIWDSRKGFIISNATYKEIGLLT | |
| | CEATVNGHLYKTNYLTHRQTNTIIDVVLSPSHGI | |
| | ELSVGEKLVLNCTARTELNVGIDENWEYPSSKH | |
| | QHKKLVNRDLKTQSGSEMKKFLSTLTIDGVTRS | |
| | DQGLYTCAASSGLMTKKNSTFVRVHEKGGGGG | |
| | SYSMTPPTLNITEESHVIDTGDSLSISCRGQHPLE | |
| | WAWPGAQEAPATGDKDSEDTGVVRDCEGTDAR | |
| | PYCKVLLLHEVHAQDTGSYVCYYKYIKARIEGT | |
| | TAASSYVFVRDFEQPFINKPDTLLVNRKDAMWV | |
| | PCLVSIPGLNVTLRSQSSVLWPDGQEVVWDDRR | |
| | GMLVSTPLLHDALYLQCETTWGDQDFLSNPFLV | |
| | HITGDKTHTCPPCPAPELLGGPSVFLFPPKPKDTL | |
| | MISRTPEVTCVVVDVSHEDPEVKFNWYVDGVE | |
| | VHNAKTKPREEQYNSTYRVVSVLTVLHQDWLN | |
| | GKEYKCKVSNKALPAPIEKTISKAKGQPREPQV | |
| | YTLPPSRDELTKNQVSLTCLVKGFYPSDIAVEWE | |
| | SNGQPENNYKTTPPVLDSDGSFFLYSKLTVDKSR | |
| | WQQGNVFSCSVMHEALHNHYTQKSLSLSPGKG | |
| | GGGGAQQEECEWDPWTCEHMGSGSATGGSGST | |
| | ASSGSGSATHQEECEWDPWTCEHMLE | |
| Exemplary | MVSYWDTGVLLCALLSCLLLTGSSSGSDTGRPF | SEQ ID NO: 63 |
| polypeptide 10 | VEMYSEIPEIIHMTEGRELVIPCRVTSPNITVTLK | |
| (as in EXG102- | KFPLDTLIPDGKRIIWDSRKGFIISNATYKEIGLLT | |
| 29) | CEATVNGHLYKTNYLTHRQTNTIIDVVLSPSHGI | |
| | ELSVGEKLVLNCTARTELNVGIDENWEYPSSKH | |
| | QHKKLVNRDLKTQSGSEMKKFLSTLTIDGVTRS | |
| | DQGLYTCAASSGLMTKKNSTFVRVHEKGGGGG | |
| | SYSMTPPTLNITEESHVIDTGDSLSISCRGQHPLE | |
| | WAWPGAQEAPATGDKDSEDTGVVRDCEGTDAR | |
| | PYCKVLLLHEVHAQDTGSYVCYYKYIKARIEGT | |
| | TAASSYVFVRDFEQPFINKPDTLLVNRKDAMWV | |
| | PCLVSIPGLNVTLRSQSSVLWPDGQEVVWDDRR | |
| | GMLVSTPLLHDALYLQCETTWGDQDFLSNPFLV | |
| | HITGDKTHTCPPCPAPELLGGPSVFLFPPKPKDTL | |
| | MISRTPEVTCVVVDVSHEDPEVKFNWYVDGVE | |
| | VHNAKTKPREEQYNSTYRVVSVLTVLHQDWLN | |
| | GKEYKCKVSNKALPAPIEKTISKAKGQPREPQV | |
| | YTLPPSRDELTKNQVSLTCLVKGFYPSDIAVEWE | |
| | SNGQPENNYKTTPPVLDSDGSFFLYSKLTVDKSR | |
| | WQQGNVFSCSVMHEALHNHYTQKSLSLSPGKG | |
| | GGGGAQGSGSATGGSGSTASSGSGSATHKFNPL | |
| | DELEETLYEQFTFQQGGGGGQEECEWDPWTCE | |
| | HMLE | |
| Exemplary | MVSYWDTGVLLCALLSCLLLTGSSSGGGGGGA | SEQ ID NO: 64 |
| polypeptide 11 | QGSGSATGGSGSTASSGSGSATHKFNPLDELEET | |
| (as in EXG102- | LYEQFTFQQGGGGGQEECEWDPWTCEHMLEGG | |
| 30) | GGGSSDTGRPFVEMYSEIPEIIHMTEGRELVIPCR | |
| | VTSPNITVTLKKFPLDTLIPDGKRIIWDSRKGFIIS | |
| | NATYKEIGLLTCEATVNGHLYKTNYLTHRQTNTI | |
| | IDVVLSPSHGIELSVGEKLVLNCTARTELNVGIDF | |
| | NWEYPSSKHQHKKLVNRDLKTQSGSEMKKFLS | |
| | TLTIDGVTRSDQGLYTCAASSGLMTKKNSTFVR | |
| | VHEKDKTHTCPPCPAPELLGGPSVFLFPPKPKDT | |
| | LMISRTPEVTCVVVDVSHEDPEVKFNWYVDGV | |
| | EVHNAKTKPREEQYNSTYRVVSVLTVLHQDWL | |
| | NGKEYKCKVSNKALPAPIEKTISKAKGQPREPQ | |
| | VYTLPPSRDELTKNQVSLTCLVKGFYPSDIAVEW | |
| | ESNGQPENNYKTTPPVLDSDGSFFLYSKLTVDKS | |
| | RWQQGNVFSCSVMHEALHNHYTQKSLSLSPGK | |
| Exemplary | MVSYWDTGVLLCALLSCLLLTGSSSGGGGGGA | SEQ ID NO: 65 |
| polypeptide 12 | QGSGSATGGSGSTASSGSGSATHKFNPLDELEET | |
| (as in EXG102- | LYEQFTFQQGGGGGQEECEWDPWTCEHMLEGG | |
| 31) | GGGSSDTGRPFVEMYSEIPEIIHMTEGRELVIPCR | |
| | VTSPNITVTLKKFPLDTLIPDGKRIIWDSRKGFIIS | |
| | NATYKEIGLLTCEATVNGHLYKTNYLTHRQTNTI | |
| | IDVVLSPSHGIELSVGEKLVLNCTARTELNVGIDF | |
| | NWEYPSSKHQHKKLVNRDLKTQSGSEMKKFLS | |
| | TLTIDGVTRSDQGLYTCAASSGLMTKKNSTFVR | |
| | VHEKGGGGGSYSMTPPTLNITEESHVIDTGDSLS | |
| | ISCRGQHPLEWAWPGAQEAPATGDKDSEDTGVV | |
| | RDCEGTDARPYCKVLLLHEVHANDTGSYVCYY | |
| | KYIKARIEGTTAASSYVFVRDFEQPFINKPDTLLV | |
| | NRKDAMWVPCLVSIPGLNVTLRSQSSVLWPDG | |
| | QEVVWDDRRGMLVSTPLLHDALYLQCETTWGD | |
| | QDFLSNPFLVHITGDKTHTCPPCPAPELLGGPSVF | |
| | LFPPKPKDTLMISRTPEVTCVVVDVSHEDPEVKF | |
| | NWYVDGVEVHNAKTKPREEQYNSTYRVVSVLT | |
| | VLHQDWLNGKEYKCKVSNKALPAPIEKTISKAK | |
| | GQPREPQVYTLPPSRDELTKNQVSLTCLVKGFYP | |
| | SDIAVEWESNGQPENNYKTTPPVLDSDGSFFLYS | |
| | KLTVDKSRWQQGNVFSCSVMHEALHNHYTQKS | |
| | LSLSPGK | |
| Exemplary | MVSYWDTGVLLCALLSCLLLTGSSSGSDTGRPF | SEQ ID NO: 66 |
| polypeptide 13 | VEMYSEIPEIIHMTEGRELVIPCRVTSPNITVTLK | |
| (as in EXG102- | KFPLDTLIPDGKRIIWDSRKGFIISNATYKEIGLLT | |
| 32) | CEATVNGHLYKTNYLTHRQTNTIIDVVLSPSHGI | |
| | ELSVGEKLVLNCTARTELNVGIDENWEYPSSKH | |
| | QHKKLVNRDLKTQSGSEMKKFLSTLTIDGVTRS | |
| | DQGLYTCAASSGLMTKKNSTFVRVHEKGGGGG | |
| | SYSMTPPTLNITEESHVIDTGDSLSISCRGQHPLE | |
| | WAWPGAQEAPATGDKDSEDTGVVRDCEGTDAR | |
| | PYCKVLLLHEVHANDTGSYVCYYKYIKARIEGT | |
| | TAASSYVFVRDFEQPFINKPDTLLVNRKDAMWV | |
| | PCLVSIPGLNVTLRSQSSVLWPDGQEVVWDDRR | |
| | GMLVSTPLLHDALYLQCETTWGDQDFLSNPFLV | |
| | HITGDKTHTCPPCPAPELLGGPSVFLFPPKPKDTL | |
| | MISRTPEVTCVVVDVSHEDPEVKFNWYVDGVE | |
| | VHNAKTKPREEQYNSTYRVVSVLTVLHQDWLN | |
| | GKEYKCKVSNKALPAPIEKTISKAKGQPREPQV | |
| | YTLPPSRDELTKNQVSLTCLVKGFYPSDIAVEWE | |
| | SNGQPENNYKTTPPVLDSDGSFFLYSKLTVDKSR | |
| | WQQGNVFSCSVMHEALHNHYTQKSLSLSPGKG | |
| | GGGGAQGSGSATGGSGSTASSGSGSATHKFNPL | |
| | DELEETLYEQFTFQQGGGGGQEECEWDPWTCE | |
| | HMLE | |

TABLE 7
Nucleic Acid Sequences for the Exemplary Fusion Proteins

| Name | Sequence | SEQ ID NO |
|---|---|---|
| Nucleic Acid of | ATGGTGTCTTATTGGGATACTGGCGTGCTGCT | SEQ ID NO: 67 |
| Exemplary | CTGTGCCCTCCTGAGTTGCCTGCTCCTGACTG | |
| Polypeptide 5 (as | GTTCTTCTTCTGGGGGCGGCGGCGGAGGCGCC | |
| in EXG102-24) | CAGCAAGAAGAGTGCGAGTGGGATCCCTGGA | |
| | CCTGCGAGCACATGGGATCCGGCAGCGCCAC | |
| | CGGAGGATCCGGAAGCACCGCCTCCAGCGGC | |
| | TCCGGCAGCGCCACCCACCAGGAGGAGTGTG | |
| | AGTGGGACCCCTGGACCTGCGAACACATGCT | |
| | GGAGGGCGGCGGCGGAGGCAGCTCCGATACT | |
| | GGGCGCCCCTTCGTGGAGATGTACTCCGAGAT | |
| | CCCTGAAATCATTCACATGACTGAGGGTCGGG | |
| | AACTGGTCATCCCATGCCGCGTGACCTCTCCC | |
| | AACATTACTGTGACCCTGAAGAAATTCCCTCT | |
| | GGACACCCTCATCCCAGATGGGAAGAGGATC | |
| | ATTTGGGACTCAAGAAAGGGTTTTATCATCAG | |
| | CAACGCTACATACAAGGAGATTGGCCTGCTCA | |
| | CCTGCGAAGCAACAGTGAACGGACACCTGTA | |
| | CAAGACTAATTATCTCACCCATAGACAGACAA | |
| | ACACTATCATTGATGTGGTCCTGTCACCAAGC | |
| | CACGGCATCGAGCTCAGCGTCGGTGAAAAGC | |
| | TGGTGCTCAATTGTACAGCCCGGACTGAGCTG | |
| | AACGTGGGCATTGACTTCAATTGGGAATACCC | |
| | CAGCTCCAAGCACCAGCATAAGAAACTGGTG | |
| | AACCGCGATCTCAAAACCCAGTCCGGATCTGA | |
| | GATGAAGAAATTTCTGAGCACCCTCACAATCG | |
| | ACGGCGTGACACGATCCGATCAGGGACTGTA | |
| | TACTTGCGCCGCTTCTAGTGGCCTGATGACCA | |
| | AGAAAAATAGCACATTCGTCAGGGTGCACGA | |
| | AAAGGGCGGCGGCGGAGGCAGCGTGTACGTG | |
| | CAAGACTACAGAAGCCCCTTCATCGCTAGCGT | |
| | GAGCGATCAGCACGGCGTGGTGTACATCACC | |
| | GAGAACAAGAACAAGACCGTGGTGATCCCCT | |
| | GCCTGGGCAGCATCAGCAACCTGAACGTGAG | |
| | CCTGTGCGCTAGATACCCCGAGAAGAGATTCG | |
| | TGCCCGACGGCAACAGAATCAGCTGGGACAG | |
| | CAAGAAGGGCTTCACCATCCCTAGCTACATGA | |
| | TCAGCTACGCCGGCATGGTGTTCTGCGAGGCC | |
| | AAGATCAACGACGAGAGCTATCAGAGCATCA | |
| | TGTACATCGTGGTCGTGGTGGGCGACAAAACT | |
| | CATACCTGCCCACCTTGTCCAGCACCAGAGCT | |
| | GCTCGGAGGACCATCCGTGTTCCTGTTTCCAC | |
| | CCAAGCCCAAAGATACTCTGATGATTTCACGC | |
| | ACACCCGAAGTCACTTGCGTGGTCGTGGACGT | |
| | GTCCCACGAGGACCCCGAAGTCAAGTTTAACT | |
| | GGTACGTGGACGGCGTCGAGGTGCATAATGC | |
| | TAAGACAAAACCCCGAGAGGAACAGTACAAC | |
| | TCTACCTATAGGGTCGTGAGTGTCCTGACAGT | |
| | GCTCCACCAGGATTGGCTGAACGGAAAGGAG | |
| | TATAAGTGCAAAGTGTCTAATAAGGCACTGCC | |
| | TGCCCCAATCGAGAAAACAATTAGTAAGGCC | |
| | AAAGGGCAGCCCAGAGAACCTCAGGTGTACA | |
| | CTCTGCCTCCATCTCGGGACGAGCTCACTAAG | |
| | AACCAGGTCAGTCTGACCTGTCTCGTGAAAGG | |
| | GTTCTATCCTAGTGATATCGCTGTGGAGTGGG | |
| | AATCAAATGGTCAGCCAGAGAACAATTACAA | |
| | GACCACACCCCCTGTCCTGGACAGCGATGGCT | |
| | CCTTCTTTCTGTATTCCAAGCTCACCGTGGAC | |
| | AAATCTCGATGGCAGCAGGGAAACGTCTTTA | |
| | GTTGTTCAGTGATGCACGAAGCCCTCCATAAC | |
| | CACTACACTCAGAAAAGCCTCAGCCTCAGCCC | |
| | TGGGAAATGA | |
| Nucleic Acid of | ATGGTGTCTTATTGGGATACTGGCGTGCTGCT | SEQ ID NO: 68 |
| Exemplary | CTGTGCCCTCCTGAGTTGCCTGCTCCTGACTG | |
| Polypeptide 6 (as | GTTCTTCTTCTGGGTCCGATACTGGGCGCCCC | |
| in EXG102-25) | TTCGTGGAGATGTACTCCGAGATCCCTGAAAT | |
| | CATTCACATGACTGAGGGTCGGGAACTGGTCA | |
| | TCCCATGCCGCGTGACCTCTCCCAACATTACT | |
| | GTGACCCTGAAGAAATTCCCTCTGGACACCCT | |
| | CATCCCAGATGGGAAGAGGATCATTTGGGAC | |
| | TCAAGAAAGGGTTTTATCATCAGCAACGCTAC | |
| | ATACAAGGAGATTGGCCTGCTCACCTGCGAA | |
| | GCAACAGTGAACGGACACCTGTACAAGACTA | |
| | ATTATCTCACCCATAGACAGACAAACACTATC | |
| | ATTGATGTGGTCCTGTCACCAAGCCACGGCAT | |
| | CGAGCTCAGCGTCGGTGAAAAGCTGGTGCTC | |
| | AATTGTACAGCCCGGACTGAGCTGAACGTGG | |
| | GCATTGACTTCAATTGGGAATACCCCAGCTCC | |
| | AAGCACCAGCATAAGAAACTGGTGAACCGCG | |
| | ATCTCAAAACCCAGTCCGGATCTGAGATGAA | |
| | GAAATTTCTGAGCACCCTCACAATCGACGGCG | |
| | TGACACGATCCGATCAGGGACTGTATACTTGC | |
| | GCCGCTTCTAGTGGCCTGATGACCAAGAAAA | |
| | ATAGCACATTCGTCAGGGTGCACGAAAAGGG | |
| | CGGCGGCGGAGGCAGCGTGTACGTGCAAGAC | |
| | TACAGAAGCCCCTTCATCGCTAGCGTGAGCGA | |
| | TCAGCACGGCGTGGTGTACATCACCGAGAAC | |
| | AAGAACAAGACCGTGGTGATCCCCTGCCTGG | |
| | GCAGCATCAGCAACCTGAACGTGAGCCTGTG | |
| | CGCTAGATACCCCGAGAAGAGATTCGTGCCC | |
| | GACGGCAACAGAATCAGCTGGGACAGCAAGA | |
| | AGGGCTTCACCATCCCTAGCTACATGATCAGC | |
| | TACGCCGGCATGGTGTTCTGCGAGGCCAAGAT | |
| | CAACGACGAGAGCTATCAGAGCATCATGTAC | |
| | ATCGTGGTCGTGGTGGGCGACAAAACTCATAC | |
| | CTGCCCACCTTGTCCAGCACCAGAGCTGCTCG | |
| | GAGGACCATCCGTGTTCCTGTTTCCACCCAAG | |
| | CCCAAAGATACTCTGATGATTTCACGCACACC | |
| | CGAAGTCACTTGCGTGGTCGTGGACGTGTCCC | |
| | ACGAGGACCCCGAAGTCAAGTTTAACTGGTA | |
| | CGTGGACGGCGTCGAGGTGCATAATGCTAAG | |
| | ACAAAACCCCGAGAGGAACAGTACAACTCTA | |
| | CCTATAGGGTCGTGAGTGTCCTGACAGTGCTC | |
| | CACCAGGATTGGCTGAACGGAAAGGAGTATA | |
| | AGTGCAAAGTGTCTAATAAGGCACTGCCTGCC | |
| | CCAATCGAGAAAACAATTAGTAAGGCCAAAG | |
| | GGCAGCCCAGAGAACCTCAGGTGTACACTCT | |
| | GCCTCCATCTCGGGACGAGCTCACTAAGAACC | |
| | AGGTCAGTCTGACCTGTCTCGTGAAAGGGTTC | |
| | TATCCTAGTGATATCGCTGTGGAGTGGGAATC | |
| | AAATGGTCAGCCAGAGAACAATTACAAGACC | |
| | ACACCCCCTGTCCTGGACAGCGATGGCTCCTT | |
| | CTTTCTGTATTCCAAGCTCACCGTGGACAAAT | |
| | CTCGATGGCAGCAGGGAAACGTCTTTAGTTGT | |
| | TCAGTGATGCACGAAGCCCTCCATAACCACTA | |
| | CACTCAGAAAAGCCTCAGCCTCAGCCCTGGG | |
| | AAAGGCGGCGGCGGAGGCGCCCAGCAAGAAG | |
| | AGTGCGAGTGGGATCCCTGGACCTGCGAGCA | |
| | CATGGGATCCGGCAGCGCCACCGGAGGATCC | |
| | GGAAGCACCGCCTCCAGCGGCTCCGGCAGCG | |
| | CCACCCACCAGGAGGAGTGTGAGTGGGACCC | |
| | CTGGACCTGCGAGCACATGCTGGAGTGA | |
| Nucleic Acid of | ATGGTGTCTTATTGGGATACTGGCGTGCTGCT | SEQ ID NO: 69 |
| Exemplary | CTGTGCCCTCCTGAGTTGCCTGCTCCTGACTG | |
| Polypeptide 7 (as | GTTCTTCTTCTGGGTCCGATACTGGGCGCCCC | |
| in EXG102-26) | TTCGTGGAGATGTACTCCGAGATCCCTGAAAT | |
| | CATTCACATGACTGAGGGTCGGGAACTGGTCA | |
| | TCCCATGCCGCGTGACCTCTCCCAACATTACT | |
| | GTGACCCTGAAGAAATTCCCTCTGGACACCCT | |
| | CATCCCAGATGGGAAGAGGATCATTTGGGAC | |
| | TCAAGAAAGGGTTTTATCATCAGCAACGCTAC | |
| | ATACAAGGAGATTGGCCTGCTCACCTGCGAA | |
| | GCAACAGTGAACGGACACCTGTACAAGACTA | |
| | ATTATCTCACCCATAGACAGACAAACACTATC | |
| | ATTGATGTGGTCCTGTCACCAAGCCACGGCAT | |
| | CGAGCTCAGCGTCGGTGAAAAGCTGGTGCTC | |
| | AATTGTACAGCCCGGACTGAGCTGAACGTGG | |
| | GCATTGACTTCAATTGGGAATACCCCAGCTCC | |
| | AAGCACCAGCATAAGAAACTGGTGAACCGCG | |
| | ATCTCAAAACCCAGTCCGGATCTGAGATGAA | |
| | GAAATTTCTGAGCACCCTCACAATCGACGGCG | |
| | TGACACGATCCGATCAGGGACTGTATACTTGC | |
| | GCCGCTTCTAGTGGCCTGATGACCAAGAAAA | |
| | ATAGCACATTCGTCAGGGTGCACGAAAAGGG | |
| | CGGCGGCGGAGGCAGCGTGTACGTGCAAGAC | |
| | TACAGAAGCCCCTTCATCGCTAGCGTGAGCGA | |
| | TCAGCACGGCGTGGTGTACATCACCGAGAAC | |
| | AAGAACAAGACCGTGGTGATCCCCTGCCTGG | |
| | GCAGCATCAGCAACCTGAACGTGAGCCTGTG | |
| | CGCTAGATACCCCGAGAAGAGATTCGTGCCC | |
| | GACGGCAACAGAATCAGCTGGGACAGCAAGA | |
| | AGGGCTTCACCATCCCTAGCTACATGATCAGC | |
| | TACGCCGGCATGGTGTTCTGCGAGGCCAAGAT | |
| | CAACGACGAGAGCTATCAGAGCATCATGTAC | |
| | ATCGTGGTCGTGGTGGGCGACAAAACTCATAC | |
| | CTGCCCACCTTGTCCAGCACCAGAGCTGCTCG | |
| | GAGGACCATCCGTGTTCCTGTTTCCACCCAAG | |
| | CCCAAAGATACTCTGATGATTTCACGCACACC | |
| | CGAAGTCACTTGCGTGGTCGTGGACGTGTCCC | |
| | ACGAGGACCCCGAAGTCAAGTTTAACTGGTA | |
| | CGTGGACGGCGTCGAGGTGCATAATGCTAAG | |
| | ACAAAACCCCGAGAGGAACAGTACAACTCTA | |
| | CCTATAGGGTCGTGAGTGTCCTGACAGTGCTC | |
| | CACCAGGATTGGCTGAACGGAAAGGAGTATA | |
| | AGTGCAAAGTGTCTAATAAGGCACTGCCTGCC | |
| | CCAATCGAGAAAACAATTAGTAAGGCCAAAG | |
| | GGCAGCCCAGAGAACCTCAGGTGTACACTCT | |
| | GCCTCCATCTCGGGACGAGCTCACTAAGAACC | |
| | AGGTCAGTCTGACCTGTCTCGTGAAAGGGTTC | |
| | TATCCTAGTGATATCGCTGTGGAGTGGGAATC | |
| | AAATGGTCAGCCAGAGAACAATTACAAGACC | |
| | ACACCCCCTGTCCTGGACAGCGATGGCTCCTT | |
| | CTTTCTGTATTCCAAGCTCACCGTGGACAAAT | |
| | CTCGATGGCAGCAGGGAAACGTCTTTAGTTGT | |
| | TCAGTGATGCACGAAGCCCTCCATAACCACTA | |
| | CACTCAGAAAAGCCTCAGCCTCAGCCCTGGG | |
| | AAAGGGGGCGGGGGCGGCGCTCAAGGCAGCG | |
| | GGAGCGCTACCGGCGGCTCCGGGAGCACAGC | |
| | TAGCAGCGGCAGCGGCTCCGCTACCCACAAG | |
| | TTCAACCCCCTGGACGAGCTGGAGGAGACCCT | |
| | CTACGAGCAGTTCACCTTCCAACAAGGCGGG | |
| | GGCGGGGGCCAAGAGGAGTGCGAGTGGGACC | |
| | CCTGGACCTGCGAGCACATGCTGGAGTGA | |
| Nucleic Acid of | ATGGTGTCTTATTGGGATACTGGCGTGCTGCT | SEQ ID NO: 70 |
| Exemplary | CTGTGCCCTCCTGAGTTGCCTGCTCCTGACTG | |
| Polypeptide 8 (as | GTTCTTCTTCTGGGGGCGGCGGCGGAGGCGCC | |
| in EXG102-27) | CAGCAAGAAGAGTGCGAGTGGGATCCCTGGA | |
| | CCTGCGAGCACATGGGATCCGGCAGCGCCAC | |
| | CGGAGGATCCGGAAGCACCGCCTCCAGCGGC | |
| | TCCGGCAGCGCCACCCACCAGGAGGAGTGTG | |
| | AGTGGGACCCCTGGACCTGCGAACACATGCT | |
| | GGAGGGCGGCGGCGGAGGCAGCTCCGATACT | |
| | GGGCGCCCCTTCGTGGAGATGTACTCCGAGAT | |
| | CCCTGAAATCATTCACATGACTGAGGGTCGGG | |
| | AACTGGTCATCCCATGCCGCGTGACCTCTCCC | |
| | AACATTACTGTGACCCTGAAGAAATTCCCTCT | |
| | GGACACCCTCATCCCAGATGGGAAGAGGATC | |
| | ATTTGGGACTCAAGAAAGGGTTTTATCATCAG | |
| | CAACGCTACATACAAGGAGATTGGCCTGCTCA | |
| | CCTGCGAAGCAACAGTGAACGGACACCTGTA | |
| | CAAGACTAATTATCTCACCCATAGACAGACAA | |
| | ACACTATCATTGATGTGGTCCTGTCACCAAGC | |
| | CACGGCATCGAGCTCAGCGTCGGTGAAAAGC | |
| | TGGTGCTCAATTGTACAGCCCGGACTGAGCTG | |
| | AACGTGGGCATTGACTTCAATTGGGAATACCC | |
| | CAGCTCCAAGCACCAGCATAAGAAACTGGTG | |
| | AACCGCGATCTCAAAACCCAGTCCGGATCTGA | |
| | GATGAAGAAATTTCTGAGCACCCTCACAATCG | |
| | ACGGCGTGACACGATCCGATCAGGGACTGTA | |
| | TACTTGCGCCGCTTCTAGTGGCCTGATGACCA | |
| | AGAAAAATAGCACATTCGTCAGGGTGCACGA | |
| | AAAGGGCGGCGGCGGAGGCAGCTACTCCATG | |
| | ACCCCCCCGACCCTGAACATCACCGAGGAGA | |
| | GCCACGTGATCGACACCGGCGACAGCCTGAG | |
| | CATCAGTTGCCGGGGCCAACACCCCCTGGAAT | |
| | GGGCATGGCCTGGGGCACAAGAGGCCCCCGC | |
| | AACGGGCGACAAGGACAGCGAGGACACCGGC | |
| | GTGGTGAGAGACTGCGAGGGCACCGACGCTA | |
| | GACCCTACTGCAAGGTGCTGCTGCTGCACGAG | |
| | GTGCACGCCCAAGACACCGGTAGCTACGTGT | |
| | GCTACTATAAGTACATCAAGGCTAGAATCGA | |
| | GGGCACCACCGCCGCTAGCAGCTACGTGTTTG | |
| | TGAGAGACTTCGAGCAGCCCTTCATCAACAAG | |
| | CCCGACACCCTGCTGGTGAACAGAAAGGACG | |
| | CCATGTGGGTGCCCTGCCTGGTGAGCATCCCC | |
| | GGCCTGAACGTGACCCTGAGATCTCAGAGCA | |
| | GCGTGCTGTGGCCCGACGGCCAAGAGGTGGT | |
| | GTGGGACGACAGAAGAGGCATGCTGGTGAGC | |
| | ACCCCCCTGCTGCACGACGCCCTGTACCTGCA | |
| | GTGCGAGACCACCTGGGGCGACCAAGACTTC | |
| | CTGAGCAACCCCTTCCTGGTGCACATCACAGG | |
| | CGACAAAACTCATACCTGCCCACCTTGTCCAG | |
| | CACCAGAGCTGCTCGGAGGACCATCCGTGTTC | |
| | CTGTTTCCACCCAAGCCCAAAGATACTCTGAT | |
| | GATTTCACGCACACCCGAAGTCACTTGCGTGG | |
| | TCGTGGACGTGTCCCACGAGGACCCCGAAGTC | |
| | AAGTTTAACTGGTACGTGGACGGCGTCGAGGT | |
| | GCATAATGCTAAGACAAAACCCCGAGAGGAA | |
| | CAGTACAACTCTACCTATAGGGTCGTGAGTGT | |
| | CCTGACAGTGCTCCACCAGGATTGGCTGAACG | |
| | GAAAGGAGTATAAGTGCAAAGTGTCTAATAA | |
| | GGCACTGCCTGCCCCAATCGAGAAAACAATT | |
| | AGTAAGGCCAAAGGGCAGCCCAGAGAACCTC | |
| | AGGTGTACACTCTGCCTCCATCTCGGGACGAG | |
| | CTCACTAAGAACCAGGTCAGTCTGACCTGTCT | |
| | CGTGAAAGGGTTCTATCCTAGTGATATCGCTG | |
| | TGGAGTGGGAATCAAATGGTCAGCCAGAGAA | |
| | CAATTACAAGACCACACCCCCTGTCCTGGACA | |
| | GCGATGGCTCCTTCTTTCTGTATTCCAAGCTCA | |
| | CCGTGGACAAATCTCGATGGCAGCAGGGAAA | |
| | CGTCTTTAGTTGTTCAGTGATGCACGAAGCCC | |
| | TCCATAACCACTACACTCAGAAAAGCCTCAGC | |
| | CTCAGCCCTGGGAAATGA | |
| Nucleic Acid of | ATGGTGTCTTATTGGGATACTGGCGTGCTGCT | SEQ ID NO: 71 |
| Exemplary | CTGTGCCCTCCTGAGTTGCCTGCTCCTGACTG | |
| polypeptide 9 (as | GTTCTTCTTCTGGGTCCGATACTGGGCGCCCC | |
| in EXG102-28) | TTCGTGGAGATGTACTCCGAGATCCCTGAAAT | |
| | CATTCACATGACTGAGGGTCGGGAACTGGTCA | |
| | TCCCATGCCGCGTGACCTCTCCCAACATTACT | |
| | GTGACCCTGAAGAAATTCCCTCTGGACACCCT | |
| | CATCCCAGATGGGAAGAGGATCATTTGGGAC | |
| | TCAAGAAAGGGTTTTATCATCAGCAACGCTAC | |
| | ATACAAGGAGATTGGCCTGCTCACCTGCGAA | |
| | GCAACAGTGAACGGACACCTGTACAAGACTA | |
| | ATTATCTCACCCATAGACAGACAAACACTATC | |
| | ATTGATGTGGTCCTGTCACCAAGCCACGGCAT | |
| | CGAGCTCAGCGTCGGTGAAAAGCTGGTGCTC | |
| | AATTGTACAGCCCGGACTGAGCTGAACGTGG | |
| | GCATTGACTTCAATTGGGAATACCCCAGCTCC | |
| | AAGCACCAGCATAAGAAACTGGTGAACCGCG | |
| | ATCTCAAAACCCAGTCCGGATCTGAGATGAA | |
| | GAAATTTCTGAGCACCCTCACAATCGACGGCG | |
| | TGACACGATCCGATCAGGGACTGTATACTTGC | |
| | GCCGCTTCTAGTGGCCTGATGACCAAGAAAA | |
| | ATAGCACATTCGTCAGGGTGCACGAAAAGGG | |
| | CGGCGGCGGAGGCAGCTACTCCATGACCCCC | |
| | CCGACCCTGAACATCACCGAGGAGAGCCACG | |
| | TGATCGACACCGGCGACAGCCTGAGCATCAG | |
| | TTGCCGGGGCCAACACCCCCTGGAATGGGCAT | |
| | GGCCTGGGGCACAAGAGGCCCCCGCAACGGG | |
| | CGACAAGGACAGCGAGGACACCGGCGTGGTG | |
| | AGAGACTGCGAGGGCACCGACGCTAGACCCT | |
| | ACTGCAAGGTGCTGCTGCTGCACGAGGTGCAC | |
| | GCCCAAGACACCGGTAGCTACGTGTGCTACTA | |
| | TAAGTACATCAAGGCTAGAATCGAGGGCACC | |
| | ACCGCCGCTAGCAGCTACGTGTTTGTGAGAGA | |
| | CTTCGAGCAGCCCTTCATCAACAAGCCCGACA | |
| | CCCTGCTGGTGAACAGAAAGGACGCCATGTG | |
| | GGTGCCCTGCCTGGTGAGCATCCCCGGCCTGA | |
| | ACGTGACCCTGAGATCTCAGAGCAGCGTGCTG | |
| | TGGCCCGACGGCCAAGAGGTGGTGTGGGACG | |
| | ACAGAAGAGGCATGCTGGTGAGCACCCCCCT | |
| | GCTGCACGACGCCCTGTACCTGCAGTGCGAGA | |
| | CCACCTGGGGCGACCAAGACTTCCTGAGCAA | |
| | CCCCTTCCTGGTGCACATCACCGGCGACAAAA | |
| | CTCATACCTGCCCACCTTGTCCAGCACCAGAG | |
| | CTGCTCGGAGGACCATCCGTGTTCCTGTTTCC | |
| | ACCCAAGCCCAAAGATACTCTGATGATTTCAC | |
| | GCACACCCGAAGTCACTTGCGTGGTCGTGGAC | |
| | GTGTCCCACGAGGACCCCGAAGTCAAGTTTAA | |
| | CTGGTACGTGGACGGCGTCGAGGTGCATAAT | |
| | GCTAAGACAAAACCCCGAGAGGAACAGTACA | |
| | ACTCTACCTATAGGGTCGTGAGTGTCCTGACA | |
| | GTGCTCCACCAGGATTGGCTGAACGGAAAGG | |
| | AGTATAAGTGCAAAGTGTCTAATAAGGCACT | |
| | GCCTGCCCCAATCGAGAAAACAATTAGTAAG | |
| | GCCAAAGGGCAGCCCAGAGAACCTCAGGTGT | |
| | ACACTCTGCCTCCATCTCGGGACGAGCTCACT | |
| | AAGAACCAGGTCAGTCTGACCTGTCTCGTGAA | |
| | AGGGTTCTATCCTAGTGATATCGCTGTGGAGT | |
| | GGGAATCAAATGGTCAGCCAGAGAACAATTA | |
| | CAAGACCACACCCCCTGTCCTGGACAGCGATG | |
| | GCTCCTTCTTTCTGTATTCCAAGCTCACCGTGG | |
| | ACAAATCTCGATGGCAGCAGGGAAACGTCTTT | |
| | AGTTGTTCAGTGATGCACGAAGCCCTCCATAA | |
| | CCACTACACTCAGAAAAGCCTCAGCCTCAGCC | |
| | CTGGGAAAGGCGGCGGCGGAGGCGCCCAGCA | |
| | AGAAGAGTGCGAGTGGGATCCCTGGACCTGC | |
| | GAGCACATGGGATCCGGCAGCGCCACCGGAG | |
| | GATCCGGAAGCACCGCCTCCAGCGGCTCCGG | |
| | CAGCGCCACCCACCAGGAGGAGTGTGAGTGG | |
| | GACCCCTGGACCTGCGAACACATGCTGGAGT | |
| | GA | |
| Nucleic Acid of | ATGGTGTCTTATTGGGATACTGGCGTGCTGCT | SEQ ID NO: 72 |
| Exemplary | CTGTGCCCTCCTGAGTTGCCTGCTCCTGACTG | |
| polypeptide 10 | GTTCTTCTTCTGGGTCCGATACTGGGCGCCCC | |
| (as in EXG102- | TTCGTGGAGATGTACTCCGAGATCCCTGAAAT | |
| 29) | CATTCACATGACTGAGGGTCGGGAACTGGTCA | |
| | TCCCATGCCGCGTGACCTCTCCCAACATTACT | |
| | GTGACCCTGAAGAAATTCCCTCTGGACACCCT | |
| | CATCCCAGATGGGAAGAGGATCATTTGGGAC | |
| | TCAAGAAAGGGTTTTATCATCAGCAACGCTAC | |
| | ATACAAGGAGATTGGCCTGCTCACCTGCGAA | |
| | GCAACAGTGAACGGACACCTGTACAAGACTA | |
| | ATTATCTCACCCATAGACAGACAAACACTATC | |
| | ATTGATGTGGTCCTGTCACCAAGCCACGGCAT | |
| | CGAGCTCAGCGTCGGTGAAAAGCTGGTGCTC | |
| | AATTGTACAGCCCGGACTGAGCTGAACGTGG | |
| | GCATTGACTTCAATTGGGAATACCCCAGCTCC | |
| | AAGCACCAGCATAAGAAACTGGTGAACCGCG | |
| | ATCTCAAAACCCAGTCCGGATCTGAGATGAA | |
| | GAAATTTCTGAGCACCCTCACAATCGACGGCG | |
| | TGACACGATCCGATCAGGGACTGTATACTTGC | |
| | GCCGCTTCTAGTGGCCTGATGACCAAGAAAA | |
| | ATAGCACATTCGTCAGGGTGCACGAAAAGGG | |
| | CGGCGGCGGAGGCAGCTACTCCATGACCCCC | |
| | CCGACCCTGAACATCACCGAGGAGAGCCACG | |
| | TGATCGACACCGGCGACAGCCTGAGCATCAG | |
| | TTGCCGGGGCCAACACCCCCTGGAATGGGCAT | |
| | GGCCTGGGGCACAAGAGGCCCCCGCAACGGG | |
| | CGACAAGGACAGCGAGGACACCGGCGTGGTG | |
| | AGAGACTGCGAGGGCACCGACGCTAGACCCT | |
| | ACTGCAAGGTGCTGCTGCTGCACGAGGTGCAC | |
| | GCCCAAGACACCGGTAGCTACGTGTGCTACTA | |
| | TAAGTACATCAAGGCTAGAATCGAGGGCACC | |
| | ACCGCCGCTAGCAGCTACGTGTTTGTGAGAGA | |
| | CTTCGAGCAGCCCTTCATCAACAAGCCCGACA | |
| | CCCTGCTGGTGAACAGAAAGGACGCCATGTG | |
| | GGTGCCCTGCCTGGTGAGCATCCCCGGCCTGA | |
| | ACGTGACCCTGAGATCTCAGAGCAGCGTGCTG | |
| | TGGCCCGACGGCCAAGAGGTGGTGTGGGACG | |
| | ACAGAAGAGGCATGCTGGTGAGCACCCCCCT | |
| | GCTGCACGACGCCCTGTACCTGCAGTGCGAGA | |
| | CCACCTGGGGCGACCAAGACTTCCTGAGCAA | |
| | CCCCTTCCTGGTGCACATCACCGGCGACAAAA | |
| | CTCATACCTGCCCACCTTGTCCAGCACCAGAG | |
| | CTGCTCGGAGGACCATCCGTGTTCCTGTTTCC | |
| | ACCCAAGCCCAAAGATACTCTGATGATTTCAC | |
| | GCACACCCGAAGTCACTTGCGTGGTCGTGGAC | |
| | GTGTCCCACGAGGACCCCGAAGTCAAGTTTAA | |
| | CTGGTACGTGGACGGCGTCGAGGTGCATAAT | |
| | GCTAAGACAAAACCCCGAGAGGAACAGTACA | |
| | ACTCTACCTATAGGGTCGTGAGTGTCCTGACA | |
| | GTGCTCCACCAGGATTGGCTGAACGGAAAGG | |
| | AGTATAAGTGCAAAGTGTCTAATAAGGCACT | |
| | GCCTGCCCCAATCGAGAAAACAATTAGTAAG | |
| | GCCAAAGGGCAGCCCAGAGAACCTCAGGTGT | |
| | ACACTCTGCCTCCATCTCGGGACGAGCTCACT | |
| | AAGAACCAGGTCAGTCTGACCTGTCTCGTGAA | |
| | AGGGTTCTATCCTAGTGATATCGCTGTGGAGT | |
| | GGGAATCAAATGGTCAGCCAGAGAACAATTA | |
| | CAAGACCACACCCCCTGTCCTGGACAGCGATG | |
| | GCTCCTTCTTTCTGTATTCCAAGCTCACCGTGG | |
| | ACAAATCTCGATGGCAGCAGGGAAACGTCTTT | |
| | AGTTGTTCAGTGATGCACGAAGCCCTCCATAA | |
| | CCACTACACTCAGAAAAGCCTCAGCCTCAGCC | |
| | CTGGGAAAGGGGGCGGGGGCGGCGCTCAAGG | |
| | CAGCGGGAGCGCTACCGGCGGCTCCGGGAGC | |
| | ACAGCTAGCAGCGGCAGCGGCTCCGCTACCC | |
| | ACAAGTTCAACCCCCTGGACGAGCTGGAGGA | |
| | GACCCTCTACGAGCAGTTCACCTTCCAACAAG | |
| | GCGGGGGCGGGGGCCAAGAGGAGTGCGAGTG | |
| | GGACCCCTGGACCTGCGAGCACATGCTGGAG | |
| | TGA | |
| Nucleic Acid of | ATGGTGTCTTATTGGGATACTGGCGTGCTGCT | SEQ ID NO: 73 |
| Exemplary | CTGTGCCCTCCTGAGTTGCCTGCTCCTGACTG | |
| polypeptide 11 | GTTCTTCTTCTGGGGGGGGCGGGGGCGGCGCT | |
| (as in EXG102- | CAAGGCAGCGGGAGCGCTACCGGCGGCTCCG | |
| 30) | GGAGCACAGCTAGCAGCGGCAGCGGCTCCGC | |
| | TACCCACAAGTTCAACCCCCTGGACGAGCTGG | |
| | AGGAGACCCTCTACGAGCAGTTCACCTTCCAA | |
| | CAAGGCGGGGGCGGGGGCCAAGAGGAGTGCG | |
| | AGTGGGACCCCTGGACCTGCGAGCACATGCT | |
| | GGAGGGCGGCGGCGGAGGCAGCTCCGATACT | |
| | GGGCGCCCCTTCGTGGAGATGTACTCCGAGAT | |
| | CCCTGAAATCATTCACATGACTGAGGGTCGGG | |
| | AACTGGTCATCCCATGCCGCGTGACCTCTCCC | |
| | AACATTACTGTGACCCTGAAGAAATTCCCTCT | |
| | GGACACCCTCATCCCAGATGGGAAGAGGATC | |
| | ATTTGGGACTCAAGAAAGGGTTTTATCATCAG | |
| | CAACGCTACATACAAGGAGATTGGCCTGCTCA | |
| | CCTGCGAAGCAACAGTGAACGGACACCTGTA | |
| | CAAGACTAATTATCTCACCCATAGACAGACAA | |
| | ACACTATCATTGATGTGGTCCTGTCACCAAGC | |
| | CACGGCATCGAGCTCAGCGTCGGTGAAAAGC | |
| | TGGTGCTCAATTGTACAGCCCGGACTGAGCTG | |
| | AACGTGGGCATTGACTTCAATTGGGAATACCC | |
| | CAGCTCCAAGCACCAGCATAAGAAACTGGTG | |
| | AACCGCGATCTCAAAACCCAGTCCGGATCTGA | |
| | GATGAAGAAATTTCTGAGCACCCTCACAATCG | |
| | ACGGCGTGACACGATCCGATCAGGGACTGTA | |
| | TACTTGCGCCGCTTCTAGTGGCCTGATGACCA | |
| | AGAAAAATAGCACATTCGTCAGGGTGCACGA | |
| | AAAGGACAAAACTCATACCTGCCCACCTTGTC | |
| | CAGCACCAGAGCTGCTCGGAGGACCATCCGT | |
| | GTTCCTGTTTCCACCCAAGCCCAAAGATACTC | |
| | TGATGATTTCACGCACACCCGAAGTCACTTGC | |
| | GTGGTCGTGGACGTGTCCCACGAGGACCCCG | |
| | AAGTCAAGTTTAACTGGTACGTGGACGGCGTC | |
| | GAGGTGCATAATGCTAAGACAAAACCCCGAG | |
| | AGGAACAGTACAACTCTACCTATAGGGTCGTG | |
| | AGTGTCCTGACAGTGCTCCACCAGGATTGGCT | |
| | GAACGGAAAGGAGTATAAGTGCAAAGTGTCT | |
| | AATAAGGCACTGCCTGCCCCAATCGAGAAAA | |
| | CAATTAGTAAGGCCAAAGGGCAGCCCAGAGA | |
| | ACCTCAGGTGTACACTCTGCCTCCATCTCGGG | |
| | ACGAGCTCACTAAGAACCAGGTCAGTCTGAC | |
| | CTGTCTCGTGAAAGGGTTCTATCCTAGTGATA | |
| | TCGCTGTGGAGTGGGAATCAAATGGTCAGCC | |
| | AGAGAACAATTACAAGACCACACCCCCTGTC | |
| | CTGGACAGCGATGGCTCCTTCTTTCTGTATTCC | |
| | AAGCTCACCGTGGACAAATCTCGATGGCAGC | |
| | AGGGAAACGTCTTTAGTTGTTCAGTGATGCAC | |
| | GAAGCCCTCCATAACCACTACACTCAGAAAA | |
| | GCCTCAGCCTCAGCCCTGGGAAATGA | |
| Nucleic Acid of | ATGGTGTCTTATTGGGATACTGGCGTGCTGCT | SEQ ID NO: 74 |
| Exemplary | CTGTGCCCTCCTGAGTTGCCTGCTCCTGACTG | |
| polypeptide 12 | GTTCTTCTTCTGGGGGGGGCGGGGGCGGCGCT | |
| (as in EXG102- | CAAGGCAGCGGGAGCGCTACCGGCGGCTCCG | |
| 31) | GGAGCACAGCTAGCAGCGGCAGCGGCTCCGC | |
| | TACCCACAAGTTCAACCCCCTGGACGAGCTGG | |
| | AGGAGACCCTCTACGAGCAGTTCACCTTCCAA | |
| | CAAGGCGGGGGCGGGGGCCAAGAGGAGTGCG | |
| | AGTGGGACCCCTGGACCTGCGAGCACATGCT | |
| | GGAGGGCGGCGGCGGAGGCAGCTCCGATACT | |
| | GGGCGCCCCTTCGTGGAGATGTACTCCGAGAT | |
| | CCCTGAAATCATTCACATGACTGAGGGTCGGG | |
| | AACTGGTCATCCCATGCCGCGTGACCTCTCCC | |
| | AACATTACTGTGACCCTGAAGAAATTCCCTCT | |
| | GGACACCCTCATCCCAGATGGGAAGAGGATC | |
| | ATTTGGGACTCAAGAAAGGGTTTTATCATCAG | |
| | CAACGCTACATACAAGGAGATTGGCCTGCTCA | |
| | CCTGCGAAGCAACAGTGAACGGACACCTGTA | |
| | CAAGACTAATTATCTCACCCATAGACAGACAA | |
| | ACACTATCATTGATGTGGTCCTGTCACCAAGC | |
| | CACGGCATCGAGCTCAGCGTCGGTGAAAAGC | |
| | TGGTGCTCAATTGTACAGCCCGGACTGAGCTG | |
| | AACGTGGGCATTGACTTCAATTGGGAATACCC | |
| | CAGCTCCAAGCACCAGCATAAGAAACTGGTG | |
| | AACCGCGATCTCAAAACCCAGTCCGGATCTGA | |
| | GATGAAGAAATTTCTGAGCACCCTCACAATCG | |
| | ACGGCGTGACACGATCCGATCAGGGACTGTA | |
| | TACTTGCGCCGCTTCTAGTGGCCTGATGACCA | |
| | AGAAAAATAGCACATTCGTCAGGGTGCACGA | |
| | AAAGGGCGGCGGCGGAGGCAGCTACTCCATG | |
| | ACCCCCCCGACCCTGAACATCACCGAGGAGA | |
| | GCCACGTGATCGACACCGGCGACAGCCTGAG | |
| | CATCAGTTGCCGGGGCCAACACCCCCTGGAAT | |
| | GGGCATGGCCTGGGGCACAAGAGGCCCCCGC | |
| | AACGGGCGACAAGGACAGCGAGGACACCGGC | |
| | GTGGTGAGAGACTGCGAGGGCACCGACGCTA | |
| | GACCCTACTGCAAGGTGCTGCTGCTGCACGAG | |
| | GTGCACGCCAACGACACCGGTAGCTACGTGT | |
| | GCTACTATAAGTACATCAAGGCTAGAATCGA | |
| | GGGCACCACCGCCGCTAGCAGCTACGTGTTTG | |
| | TGAGAGACTTCGAGCAGCCCTTCATCAACAAG | |
| | CCCGACACCCTGCTGGTGAACAGAAAGGACG | |
| | CCATGTGGGTGCCCTGCCTGGTGAGCATCCCC | |
| | GGCCTGAACGTGACCCTGAGATCTCAGAGCA | |
| | GCGTGCTGTGGCCCGACGGCCAAGAGGTGGT | |
| | GTGGGACGACAGAAGAGGCATGCTGGTGAGC | |
| | ACCCCCCTGCTGCACGACGCCCTGTACCTGCA | |
| | GTGCGAGACCACCTGGGGCGACCAAGACTTC | |
| | CTGAGCAACCCCTTCCTGGTGCACATCACAGG | |
| | CGACAAAACTCATACCTGCCCACCTTGTCCAG | |
| | CACCAGAGCTGCTCGGAGGACCATCCGTGTTC | |
| | CTGTTTCCACCCAAGCCCAAAGATACTCTGAT | |
| | GATTTCACGCACACCCGAAGTCACTTGCGTGG | |
| | TCGTGGACGTGTCCCACGAGGACCCCGAAGTC | |
| | AAGTTTAACTGGTACGTGGACGGCGTCGAGGT | |
| | GCATAATGCTAAGACAAAACCCCGAGAGGAA | |
| | CAGTACAACTCTACCTATAGGGTCGTGAGTGT | |
| | CCTGACAGTGCTCCACCAGGATTGGCTGAACG | |
| | GAAAGGAGTATAAGTGCAAAGTGTCTAATAA | |
| | GGCACTGCCTGCCCCAATCGAGAAAACAATT | |
| | AGTAAGGCCAAAGGGCAGCCCAGAGAACCTC | |
| | AGGTGTACACTCTGCCTCCATCTCGGGACGAG | |
| | CTCACTAAGAACCAGGTCAGTCTGACCTGTCT | |
| | CGTGAAAGGGTTCTATCCTAGTGATATCGCTG | |
| | TGGAGTGGGAATCAAATGGTCAGCCAGAGAA | |
| | CAATTACAAGACCACACCCCCTGTCCTGGACA | |
| | GCGATGGCTCCTTCTTTCTGTATTCCAAGCTCA | |
| | CCGTGGACAAATCTCGATGGCAGCAGGGAAA | |
| | CGTCTTTAGTTGTTCAGTGATGCACGAAGCCC | |
| | TCCATAACCACTACACTCAGAAAAGCCTCAGC | |
| | CTCAGCCCTGGGAAATGA | |
| Nucleic Acid of | ATGGTGTCTTATTGGGATACTGGCGTGCTGCT | SEQ ID NO: 75 |
| Exemplary | CTGTGCCCTCCTGAGTTGCCTGCTCCTGACTG | |
| polypeptide 13 | GTTCTTCTTCTGGGTCCGATACTGGGCGCCCC | |
| (as in EXG102- | TTCGTGGAGATGTACTCCGAGATCCCTGAAAT | |
| 32) | CATTCACATGACTGAGGGTCGGGAACTGGTCA | |
| | TCCCATGCCGCGTGACCTCTCCCAACATTACT | |
| | GTGACCCTGAAGAAATTCCCTCTGGACACCCT | |
| | CATCCCAGATGGGAAGAGGATCATTTGGGAC | |
| | TCAAGAAAGGGTTTTATCATCAGCAACGCTAC | |
| | ATACAAGGAGATTGGCCTGCTCACCTGCGAA | |
| | GCAACAGTGAACGGACACCTGTACAAGACTA | |
| | ATTATCTCACCCATAGACAGACAAACACTATC | |
| | ATTGATGTGGTCCTGTCACCAAGCCACGGCAT | |
| | CGAGCTCAGCGTCGGTGAAAAGCTGGTGCTC | |
| | AATTGTACAGCCCGGACTGAGCTGAACGTGG | |
| | GCATTGACTTCAATTGGGAATACCCCAGCTCC | |
| | AAGCACCAGCATAAGAAACTGGTGAACCGCG | |
| | ATCTCAAAACCCAGTCCGGATCTGAGATGAA | |
| | GAAATTTCTGAGCACCCTCACAATCGACGGCG | |
| | TGACACGATCCGATCAGGGACTGTATACTTGC | |
| | GCCGCTTCTAGTGGCCTGATGACCAAGAAAA | |
| | ATAGCACATTCGTCAGGGTGCACGAAAAGGG | |
| | CGGCGGCGGAGGCAGCTACTCCATGACCCCC | |
| | CCGACCCTGAACATCACCGAGGAGAGCCACG | |
| | TGATCGACACCGGCGACAGCCTGAGCATCAG | |
| | TTGCCGGGGCCAACACCCCCTGGAATGGGCAT | |
| | GGCCTGGGGCACAAGAGGCCCCCGCAACGGG | |
| | CGACAAGGACAGCGAGGACACCGGCGTGGTG | |
| | AGAGACTGCGAGGGCACCGACGCTAGACCCT | |
| | ACTGCAAGGTGCTGCTGCTGCACGAGGTGCAC | |
| | GCCAACGACACCGGTAGCTACGTGTGCTACTA | |
| | TAAGTACATCAAGGCTAGAATCGAGGGCACC | |
| | ACCGCCGCTAGCAGCTACGTGTTTGTGAGAGA | |
| | CTTCGAGCAGCCCTTCATCAACAAGCCCGACA | |
| | CCCTGCTGGTGAACAGAAAGGACGCCATGTG | |
| | GGTGCCCTGCCTGGTGAGCATCCCCGGCCTGA | |
| | ACGTGACCCTGAGATCTCAGAGCAGCGTGCTG | |
| | TGGCCCGACGGCCAAGAGGTGGTGTGGGACG | |
| | ACAGAAGAGGCATGCTGGTGAGCACCCCCCT | |
| | GCTGCACGACGCCCTGTACCTGCAGTGCGAGA | |
| | CCACCTGGGGCGACCAAGACTTCCTGAGCAA | |
| | CCCCTTCCTGGTGCACATCACAGGCGACAAAA | |
| | CTCATACCTGCCCACCTTGTCCAGCACCAGAG | |
| | CTGCTCGGAGGACCATCCGTGTTCCTGTTTCC | |
| | ACCCAAGCCCAAAGATACTCTGATGATTTCAC | |
| | GCACACCCGAAGTCACTTGCGTGGTCGTGGAC | |
| | GTGTCCCACGAGGACCCCGAAGTCAAGTTTAA | |
| | CTGGTACGTGGACGGCGTCGAGGTGCATAAT | |
| | GCTAAGACAAAACCCCGAGAGGAACAGTACA | |
| | ACTCTACCTATAGGGTCGTGAGTGTCCTGACA | |
| | GTGCTCCACCAGGATTGGCTGAACGGAAAGG | |
| | AGTATAAGTGCAAAGTGTCTAATAAGGCACT | |
| | GCCTGCCCCAATCGAGAAAACAATTAGTAAG | |
| | GCCAAAGGGCAGCCCAGAGAACCTCAGGTGT | |
| | ACACTCTGCCTCCATCTCGGGACGAGCTCACT | |
| | AAGAACCAGGTCAGTCTGACCTGTCTCGTGAA | |
| | AGGGTTCTATCCTAGTGATATCGCTGTGGAGT | |
| | GGGAATCAAATGGTCAGCCAGAGAACAATTA | |
| | CAAGACCACACCCCCTGTCCTGGACAGCGATG | |
| | GCTCCTTCTTTCTGTATTCCAAGCTCACCGTGG | |
| | ACAAATCTCGATGGCAGCAGGGAAACGTCTTT | |
| | AGTTGTTCAGTGATGCACGAAGCCCTCCATAA | |
| | CCACTACACTCAGAAAAGCCTCAGCCTCAGCC | |
| | CTGGGAAAGGGGGCGGGGGCGGCGCTCAAGG | |
| | CAGCGGGAGCGCTACCGGCGGCTCCGGGAGC | |
| | ACAGCTAGCAGCGGCAGCGGCTCCGCTACCC | |
| | ACAAGTTCAACCCCCTGGACGAGCTGGAGGA | |
| | GACCCTCTACGAGCAGTTCACCTTCCAACAAG | |
| | GCGGGGGCGGGGGCCAAGAGGAGTGCGAGTG | |
| | GGACCCCTGGACCTGCGAGCACATGCTGGAG | |
| | TGA | |

### 6.5. Example 5—Constructions of AAV Vectors Comprising the Nucleic Acid Encoding the Fusion Proteins Comprising an Additional Trap C Domain

Nucleic acid sequences encoding the exemplary fusion proteins described in Section 6.4 were introduced into exemplary rAAV vectors, e.g., derived from rAAV2 and rAAV8 in this example to generate rAAV vectors as shown in FIG. 8 and FIGS. 9A-9I.

### 6.6. Example 6-Protein Expressions, Ligand Binding and Other Functional Assays

HEK293 cells are transiently transfected with plasmid constructs or infected with AAV vectors described above. Transgene expression is analyzed and compared. Various in vitro and in vivo assays are performed for these constructs.

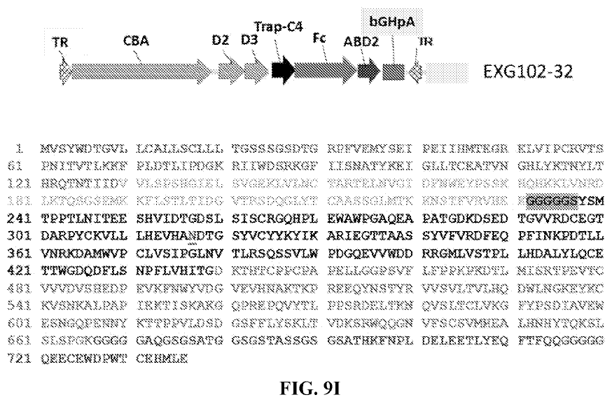

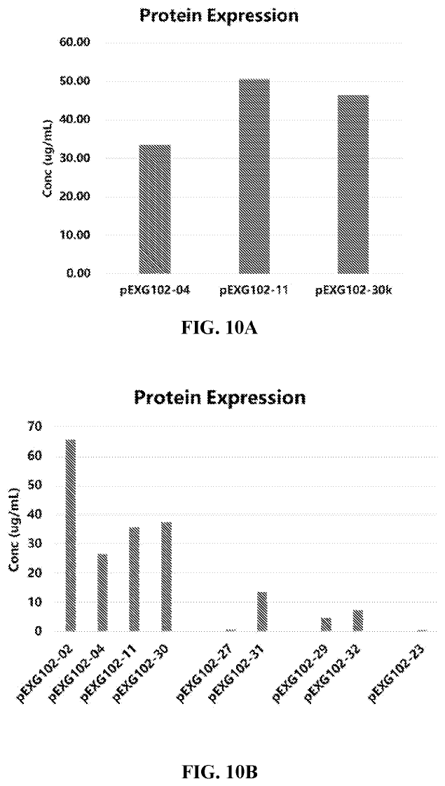

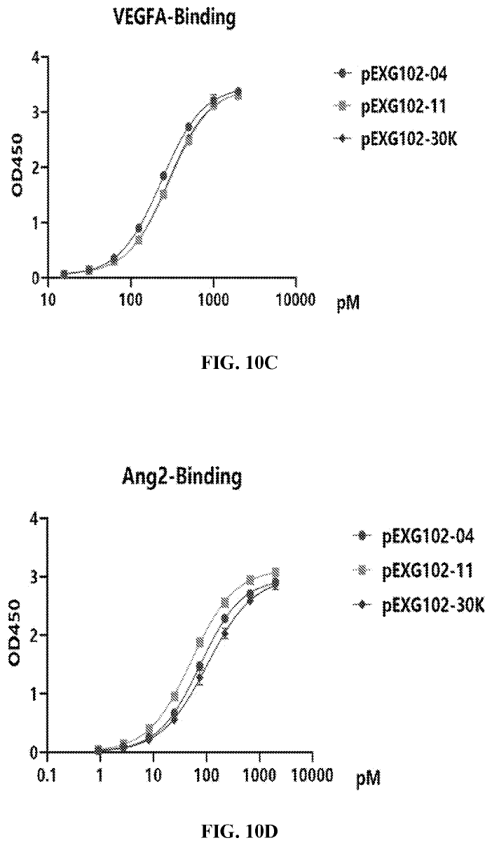

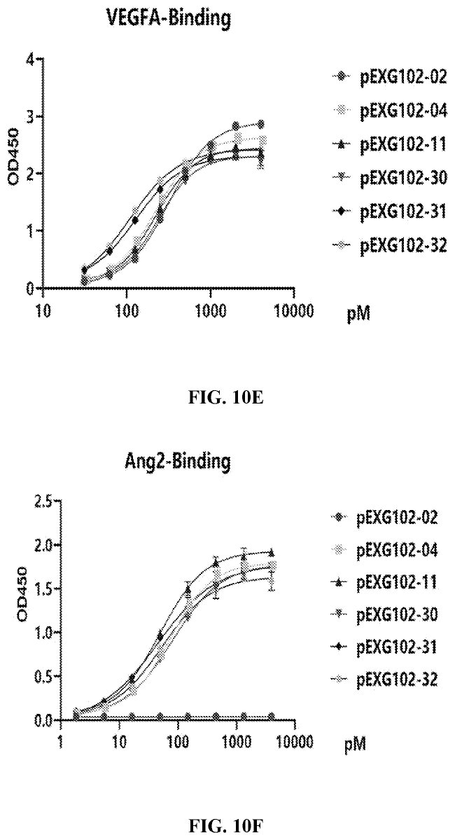

As shown in FIGS. 10A-10F, constructs containing ABD2 are comparable to the constructs with ABD domain in protein expression, VEGF-A binding or Ang2 binding.

### 6.7. Example 7-Effect of the ANG-2 Binding Domain on the Inhibition of CNV Lesion in the CNV Mouse Model

The FFA mean score was measured in eyes treated with AAV-GFP (negative control), EXG102-02 (positive control), EXG102-04, EXG102-09, EXG102-10, or EXG102-11 using the CNV mouse model on days 29 and 36 post injection. The angiograms were graded as follows: score 0, no staining; score 1, slightly stained; score 2, moderately stained; and score 3, strongly stained.

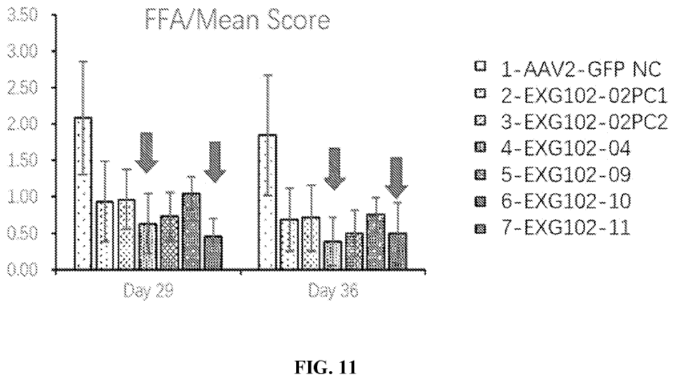

FIG. 11 shows the compound effect of the ANG-2 binding domain on the inhibition of CNV lesion when compared to the positive controls that have the VEGF inhibitor only FXG102-04 and FXG102-11 showed most effective inhibition on the CNV lesion (four wide arrows).

### 6.8. Example 8—No Grade 3 CNV Lesion in Eyes Treated with EXG102-04, EXG102-09, EXG102-10 or EXG102-11 in the CNV Mouse Model

The FFA mean score was measured in eyes treated with AAV-GFP (negative control), EXG102-02 (positive control), EXG102-04, EXG102-09, EXG102-10, or EXG102-11 on days 29 and 36 post injection. The angiograms were graded as follows: score 0, no staining; score 1, slightly stained; score 2, moderately stained: and score 3, strongly stained.

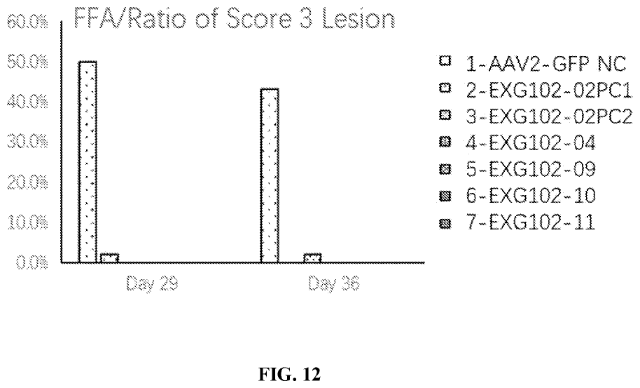

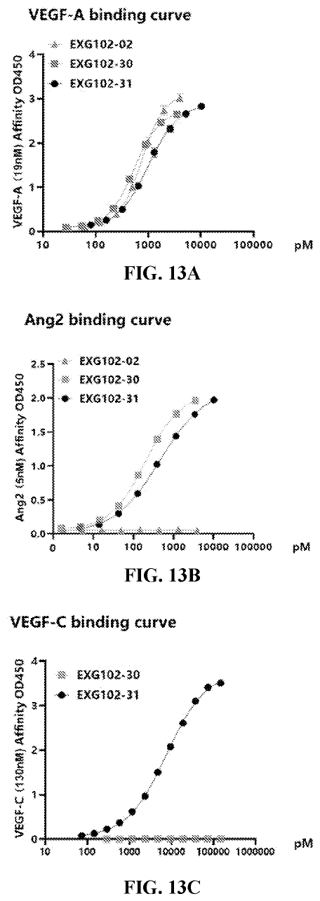

As shown in FIG. 12, there is no Grade 3 CNV lesion found in any eyes treated with EXG102-04, EXG102-09, EXG102-10 or EXG102-11, while 40-50% Grade 3 lesion were found in eyes received AAV2-GFP (negative control).

### 6.9. Example 9-EXG102-31 Binds to VEGF-A, Ang2 and VEGF-C

In vitro VEGF-A, Ang2, or VEGF-C binding affinity of the transgene products expressed from HEK293T cells was measured. HEK293T cells were transfected with plasmid DNA of pEXG102-02, pEXG102-30 or pEXG102-31. The target protein expressed were affinity purified from the cell lysate. Binding capability of EXG102-02, EXG102-30 and EXG102-31 to VEGF-A, Ang2, and VEGF-C was measured by ELISA, respectively.

FIGS. 12A-12C shows that EXG102-31 was able to bind to each of VEGF-A, Ang2 and VEGF-C. Surprisingly, the VEGF-A binding affinity of EXG102-31 is similar to positive control EXG102-02.

### 6.10. Example 10-Score of CNV Lesions Decreased in the Treated Groups in the CNV Mouse Model

The FFA mean score was measured in eyes treated with vehicle control, EXG102-02, EXG102-30, or EXG102-31. Animals were given intraperitoneal injection of Fluorescein Sodium (100 mg/ml, 30 μL/animal) before fluorescein angiography. The FFA images were taken 3 minutes post injection. The angiograms were graded as follows: score 0, no staining: score 1, slightly stained; score 2, moderately stained; and score 3, strongly stained. Rate of Score 3 lesions and the mean score were calculated.

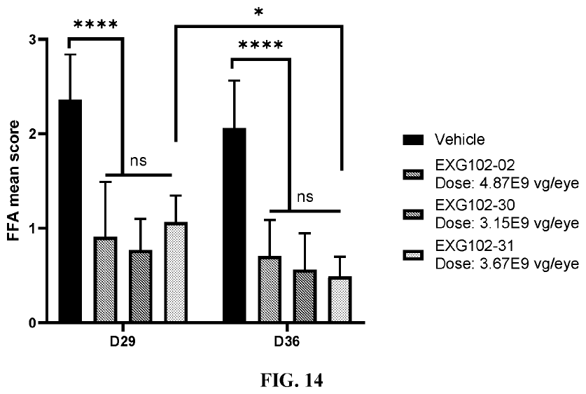

FIG. 14 shows the FFA mean score in laser-induced CNV mouse model. Compared with vehicle-treated group, the scores of CNV lesions in all treated groups decreased significantly (p≤0.001) on each of days 29 and 36. Although the scores of CNV lesions in the EXG102-31-treated group is higher than that of the EXG102-02 and EXG102-30-treated groups on day 29, there was no significant difference between test-article treated groups (p>0.05) on day 36, which is possibly due to high variation among individual mouse. Especially, comparing with Day 29, the mean of volumes of CNV lesions in EXG102-31-treated group decreased significantly on Day 36 (p≤0.05), suggesting that the compound effect of the VEGF-C binding domain in EXG102-31 on the inhibition of late stage CNV lesion. FIG. 14 suggests that EXG102-31 has advantage over EXG102-30 for long-term CNV inhibition, as the CNV lesion score significantly improved from day 29 to day 36 when compared to that of EXG102-30. According to Cabral, T., et al. Ophthalmol Retina, 2018. 2(1): p. 31-37, the level of VEGF-C and Ang-2 increased significantly after VEGF-A is inhibited. EXG102-31 is able to neutralize almost all VEGF subtypes including VEGF-C, as well as Ang-2.

### 6.11. Example 11—No Grade 3 CNV Lesion in Eyes Treated with EXG102-02, EXG102-30, or EXG102-31

The ratio of Grade 3 CNV lesions were measured in eyes treated with vehicle control, EXG102-02, EXG102-30, or EXG102-31. Animals were given intraperitoneal injection of Fluorescein Sodium (100 mg/ml, 30 μL/animal) before fluorescein angiography. The FFA images were taken 3 minutes post injection. The angiograms were graded as follows: score 0, no staining; score 1, slightly stained; score 2, moderately stained; and score 3, strongly stained. Rate of Score 3 lesions and the mean score were calculated.

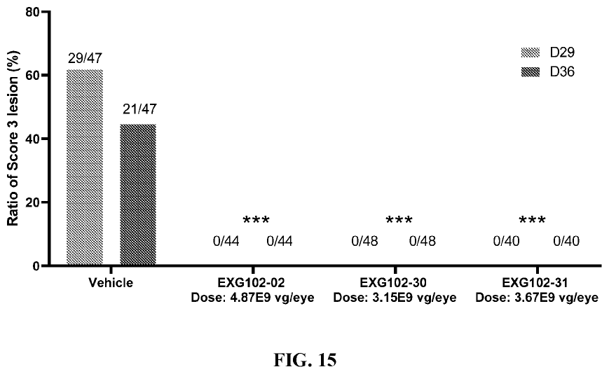

As shown in FIG. 15, there is no Grade 3 CNV lesion found in any eyes treated with EXG102-02, EXG102-30, or EXG102-31, while 29 and 21 out of 47 CNV Grade 3 lesions were found in eyes received vehicle control.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed:

1. A polypeptide comprising:
(i) a first domain that binds to VEGF;
(ii) a second domain that binds to VEGF; and
(iii) a third domain that binds to an angiopoietin,
wherein optionally the third domain is at the N-terminus of the first domain and the second domain.

2. The polypeptide of claim 1, wherein the first domain is derived from VEGF receptor-1 (VEGFR-1 or FLT-1).

3. The polypeptide of claim 2, wherein the first domain comprises domain 2 of VEGFR-1 or a variant thereof.

4. The polypeptide of claim 3, wherein the first domain comprises or consists of an amino acid sequence of SEQ ID NO: 1.

5. The polypeptide of claim 3, wherein the first domain comprises or consists of an amino acid sequence having at least 80%, 85%, 90%, 91%, 92%, 93%, 94%, 95%, 96%, 97%, 98%, 99% identify to SEQ ID NO: 1.

6. The polypeptide of any one of claims 1 to 5, wherein the second domain is derived from VEGF receptor-2 (VEGFR-2 or Flk-1).

7. The polypeptide of claim 6, wherein the second domain comprises domain 3 of VEGFR-2 or a variant thereof.

8. The polypeptide of claim 7, wherein the second domain comprises or consists of an amino acid sequence of SEQ ID NO: 2.

9. The polypeptide of claim 7, wherein the second domain comprises or consists of an amino acid sequence having at least 80%, 85%, 90%, 91%, 92%, 93%, 94%, 95%, 96%, 97%, 98%, 99% identify to SEQ ID NO: 2.

10. The polypeptide of any one of claims 1 to 9, wherein the third domain comprises one or two repeats of an amino acid sequence of SEQ ID NO: 3; and/or one or two repeats of an amino acid sequence of SEQ ID NO: 51, wherein optionally (i) the third domain comprises two repeats of the amino acid sequence of SEQ ID NO: 3, (ii) the third domain comprises two repeats of the amino acid sequence of SEQ ID NO: 51; or (iii) the third domain comprises an amino acid sequence of SEQ ID NO: 3 and an amino acid sequence of SEQ ID NO: 51.

11. The polypeptide of any one of claims 1 to 9, wherein (i) the third domain comprises or consists of an amino acid sequence of SEQ ID NO: 4, or the third domain comprises an amino acid sequence having at least 80%, 85%, 90%, 91%, 92%, 93%, 94%, 95%, 96%, 97%, 98%, 99% identify to SEQ ID NO: 4; (ii) the third domain comprises or consists of an amino acid sequence of SEQ ID NO: 52, or the third domain comprises an amino acid sequence having at least 80%, 85%, 90%, 91%, 92%, 93%, 94%, 95%, 96%, 97%, 98%, 99% identify to SEQ ID NO: 52; (iii) the third domain comprises or consists of an amino acid sequence of SEQ ID NO: 53, or the third domain comprises an amino acid sequence having at least 80%, 85%, 90%, 91%, 92%, 93%, 94%, 95%, 96%, 97%, 98%, 99% identify to SEQ ID NO: 53; or (iv) the third domain comprises or consists of an amino acid sequence of SEQ ID NO: 54, or the third domain comprises an amino acid sequence having at least 80%, 85%, 90%, 91%, 92%, 93%, 94%, 95%, 96%, 97%, 98%, 99% identify to SEQ ID NO: 54.

12. The polypeptide of any one of claims 1 to 11, wherein the third domain is at the N-terminus of the first domain and the second domain.

13. A polypeptide comprising:
(i) a first domain that binds to VEGF, the first domain comprising an amino acid sequence having at least 80%, 85%, 90%, 91%, 92%, 93%, 94%, 95%, 96%, 97%, 98%, 99% or 100% identify to SEQ ID NO: 1;
(ii) a second domain that binds to VEGF, the second domain comprising an amino acid sequence having at least 80%, 85%, 90%, 91%, 92%, 93%, 94%, 95%, 96%, 97%, 98%, 99% or 100% identify to SEQ ID NO: 2; and
(iii) a third domain that binds to an angiopoietin, the third domain comprising two amino acid sequences each comprising an amino acid sequence having at least 80%, 85%, 90%, or 100% identify to SEQ ID NO: 3; two amino acid sequences each comprising an amino acid sequence having at least 80%, 85%, 90%, or 100% identify to SEQ ID NO: 51; or one amino acid sequence having at least 80%, 85%, 90%, or 100% identify to SEQ ID NO: 3 and one amino acid sequence having at least 80%, 85%, 90%, or 100% identify to SEQ ID NO: 51.

14. The polypeptide of any one of claims 1 to 13, further comprising a Fc region of an antibody or a variant thereof.

15. The polypeptide of claim 14, wherein the Fc region comprises an amino acid sequence of SEQ ID NO: 5.

16. The polypeptide of any one of claims 1 to 15, further comprising a signal peptide, wherein optionally the signal peptide comprises an amino acid sequence of SEQ ID NO: 6.

17. The polypeptide of any one of claims 1 to 17, further comprises one or more linkers.

18. A polypeptide comprising an amino acid sequence of SEQ ID NO: 7, SEQ ID NO: 8, SEQ ID NO: 9, or SEQ ID NO: 10, or an amino acid sequence having at least 80%, 85%, 90%, 91%, 92%, 93%, 94%, 95%, 96%, 97%, 98%, 99% identify to SEQ ID NO: 7, SEQ ID NO: 8, SEQ ID NO: 9, or SEQ ID NO: 10.

19. The polypeptide of any one of claims 1 to 18, wherein the polypeptide further comprises a VEGFC binding domain.

20. The polypeptide of claim 19, wherein the VEGFC binding domain is derived from VEGFR-2.

21. The polypeptide of claim 19, wherein the VEGFC binding domain is derived from VEGF receptor-3 (VEGFR-3).

22. The polypeptide of claim 19, wherein the VEGFC binding domain comprises an amino acid sequence having at least 80%, 85%, 90%, 91%, 92%, 93%, 94%, 95%, 96%, 97%, 98%, 99% or 100% identify to SEQ ID NO: 55.

23. The polypeptide of claim 19, wherein the VEGFC binding domain comprises an amino acid sequence having at least 80%, 85%, 90%, 91%, 92%, 93%, 94%, 95%, 96%, 97%, 98%, 99% or 100% identify to SEQ ID NO: 56; or wherein the VEGFC binding domain comprises an amino acid sequence having at least 80%, 85%, 90%, 91%, 92%, 93%, 94%, 95%, 96%, 97%, 98%, 99% or 100% identify to SEQ ID NO: 57.

24. A polypeptide comprising an amino acid sequence of SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 or SEQ ID NO: 66, or an amino acid sequence having at least 80%, 85%, 90%, 91%, 92%, 93%, 94%, 95%, 96%, 97%, 98%, 99% identify to SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, or SEQ ID NO: 66.

25. The polypeptide of any one of claims 1 to 24, wherein the polypeptide is genetically fused or chemically conjugated to an agent.

26. An isolated nucleic acid comprising a nucleic acid sequence encoding the polypeptide of any one of claims 1 to 24.

27. A vector comprising the isolated nucleic acid of claim 26.

28. The vector of claim 27, wherein the vector is a viral vector.

29. The vector of claim 28, wherein the viral vector is an adeno-associated virus (AAV) vector.

30. The vector of claim 29, wherein the AAV vector is derived from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a combination or variant thereof.

31. The vector of claim 30, wherein the vector is a recombinant AAV2 (rAAV2) vector, a recombinant AAV8 (rAAV8) vector, or a variant thereof.

32. A recombinant AAV (rAAV) vector comprising a nucleic acid encoding a polypeptide wherein the polypeptide comprises (i) a first domain derived from VEGFR-1; (ii) a second domain derived from VEGFR-2; and (iii) a third domain that is capable of binding to an angiopoietin, wherein the rAAV vector comprises an inverted terminal repeat (ITR) from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, or AAV44-9.

33. A recombinant AAV (rAAV) vector comprising a nucleic acid encoding a polypeptide wherein the polypeptide comprises (i) a first domain derived from VEGFR-1; (ii) a second domain derived from VEGFR-2; (iii) a third domain that is capable of binding to an angiopoietin; and (iv) a fourth domain that is capable of binding to VEGFC, wherein the rAAV vector comprises an inverted terminal repeat (ITR) from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, or AAV44-9.

34. The rAAV vector of claim 32 or claim 33, wherein the ITR is from AAV2.

35. The rAAV vector of claim 32 or claim 33, wherein the ITR is from AAV8.

36. The rAAV vector of any one of claims 32 to 35, wherein the third domain is at N-terminus of the first domain and the second domain.

37. A vector comprising a nucleic acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23, or a nucleic acid sequence having at least 80%, 85%, 90%, 91%, 92%, 93%, 94%, 95%, 96%, 97%, 98%, 99% identify to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23.

38. A vector comprising a nucleic acid sequence of SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75, or a nucleic acid sequence having at least 80%, 85%, 90%, 91%, 92%, 93%, 94%, 95%, 96%, 97%, 98%, 99% identify to SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75.

39. A recombinant AAV (rAAV) particle comprising
(a) a nucleic acid encoding a polypeptide wherein the polypeptide comprises (i) a first domain derived from VEGFR-1; (ii) a second domain derived from VEGFR-2; and (iii) a third domain that is capable of binding to an angiopoietin; and
(b) a capsid protein of AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a variant thereof.

40. A recombinant AAV (rAAV) particle comprising
(a) a nucleic acid encoding a polypeptide wherein the polypeptide comprises (i) a first domain derived from VEGFR-1; (ii) a second domain derived from VEGFR-2; and (iii) a third domain that is capable of binding to an angiopoietin; and (iv) a fourth domain capable of binding to VEGFC; and
(b) a capsid protein of AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a variant thereof.

41. The rAAV particle of claim 39 or claim 40, wherein the third domain is at N-terminus of the first domain and the second domain.

42. The rAAV particle of any one of claims 39 to 41, wherein the capsid protein is a AAV2 capsid protein.

43. The rAAV particle of any one of claims 39 to 41, wherein the capsid protein is a AAV8 capsid protein.

44. The rAAV particle of any one of claims 39 to 41, wherein the capsid protein is a variant of a AAV2 capsid protein comprising an amino acid sequence of SEQ ID NO: 48, wherein the variant of the AAV2 capsid protein comprises the amino acid substitutions of Y444F, R487G, T491V, Y500F, R585S, R588T, and Y730F of capsid protein VP1 of AAV2.

45. A pharmaceutical composition comprising the polypeptide of any one of claims 1 to 25, the vector or rAAV vector of any one of claims 27 to 38, or the rAAV particle of any one of claims 39 to 44, and a pharmaceutically acceptable excipient.

46. A method of treating a disease or disorder in a subject, comprising administering to the subject the polypeptide of any one of claims 1 to 25, the vector or rAAV of any one of claims 27 to 38, the rAAV particle of any one of claims 39 to 44, or the pharmaceutical composition of claim 45.

47. The method of claim 46, wherein the disease or disorder is an angiogenic or neovascular disease or disorder.

48. The method of claim 46, wherein the disease or disorder is an inflammatory disease, ocular disease, autoimmune disease, or cancer.

49. The method of claim 46, wherein the disease or disorder is an eye disease or disorder.

50. The method of claim 49, wherein the eye disease or disorder is selected from a group consisting of uveitis, retinitis pigmentosa, neovascular glaucoma, diabetic retinopathy (DR) (including proliferative diabetic retinopathy), ischemic retinopathy, intraocular neovascularization, age-related macular degeneration (AMD), retinal neovascularization, diabetic macular edema (DME), diabetic retina ischemia, diabetic retinal edema, retinal vein occlusion (including central retinal vein occlusion and branched retinal vein occlusion), macular edema, and macular edema following retinal vein occlusion (RVO).

51. The method of claim 50, wherein the disease or disorder is age-related macular degeneration (AMD).

52. The method of claim 51, wherein the AMD is wet AMD (wAMD).

53. The method of any one of 46 to 52, wherein the method comprises administering by intravitreal or subretinal injection into an eye of the subject.


SEQUENCE LISTING (75 sequences)

SEQ ID NO: 1 - protein, 103 aa, Artificial Sequence
  Exemplary first domain derived from VEGFR-1 (D2)
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIID

SEQ ID NO: 2 - protein, 101 aa, Artificial Sequence
  Exemplary second domain derived from VEGFR-2 (D3)
VVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHE

SEQ ID NO: 3 - protein, 14 aa, Artificial Sequence
  14 amino acid sequence in exemplary third domain (ABD), ABD (Con4)
QEECEWDPWTCEHM

SEQ ID NO: 4 - protein, 59 aa, Artificial Sequence
  Exemplary third domain (ABD), ABD (1) (2xCon4)
GGGGGAQQEECEWDPWTCEHMGSGSATGGSGSTASSGSGSATHQEECEWDPWTCEHMLE

SEQ ID NO: 5 - protein, 227 aa, Artificial Sequence
  Exemplary IgG-Fc (1)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 26 aa, Artificial Sequence
  Flt-1 Signal
MVSYWDTGVLLCALLSCLLLTGSSSG

SEQ ID NO: 7 - protein, 517 aa, Artificial Sequence
  Exemplary polypeptide 1
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGAQQEECEWDPWTCEHMGSGSATGGSGSTASSGSGSATHQEECEWDPWTCEHMLE

SEQ ID NO: 8 - protein, 523 aa, Artificial Sequence
  Exemplary polypeptide 2
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGGSGGGGGAQQEECEWDPWTCEHMGSGSATGGSGSTASSGSGSATHQEECEWDPWTCEHMLEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 529 aa, Artificial Sequence
  Exemplary polypeptide 3
MVSYWDTGVLLCALLSCLLLTGSSSGGGGGGSGGGGGAQQEECEWDPWTCEHMGSGSATGGSGSTASSGSGSATHQEECEWDPWTCEHMLEGGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 316 aa, Artificial Sequence
  Exemplary polypeptide 4
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVGGGGGSGGGGGAQQEECEWDPWTCEHMGSGSATGGSGSTASSGSGSATHQEECEWDPWTCEHMLE

SEQ ID NO: 11 - DNA, 3672 bp, Artificial Sequence
  EXG102-02
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccaccatggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaaggacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatgataagcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 12 - DNA, 4348 bp, Artificial Sequence
  EXG102-03-01
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgagagaattcgccaccatggtgagctattgggatacaggcgtgctgctgtgcgccctgctgagctgcctgctgctgaccggcagcagcagcggcagcgacaccggcagacctttcgtggagatgtacagcgagatccctgagatcatccacatgaccgagggcagagagctggtgatcccttgcagagtgaccagccctaatatcaccgtgaccctcaagaagttccctctggataccctgatccctgacggcaagagaatcatctgggacagcagaaagggcttcatcatcagcaatgccacctacaaggagatcggcctgctgacctgcgaggccaccgtgaatggccacctgtacaagaccaattacctgacccacagacagaccaataccatcatcgacgtggtgctgagccctagccacggcatcgagctgagcgtgggcgagaagctggtgctgaattgcaccgccagaaccgagctgaatgtgggcatcgacttcaattgggagtaccctagcagcaagcaccagcacaagaagctggtgaatagagacctgaagacccagagcggcagcgagatgaagaaattcctgagcaccctgaccatcgacggcgtgaccagaagcgaccagggcctgtacacctgcgctgccagcagcggcctgatgaccaagaagaatagcaccttcgtgagagtgcacgagaaggacaagacccacacctgccctccttgccctgcccctgagctgctgggcggccctagcgtgttcctgttccctcctaagcctaaggacaccctcatgatcagcagaacccctgaggtgacctgcgtggtggtggacgtgagccacgaggaccctgaggtgaagttcaattggtacgtggacggcgtggaggtgcacaatgccaagaccaagcctagagaggagcagtacaatagcacctacagagtggtgagcgtgctgaccgtgctgcaccaggactggctgaatggcaaggagtacaagtgcaaggtgagcaataaggccctgcctgcccctatcgagaagaccatcagcaaggccaagggccagcctagagagcctcaggtgtacaccctgcctcctagcagagacgagctgaccaagaatcaggtgagcctgacctgcctggtgaagggcttctaccctagcgacatcgccgtggagtgggagagcaatggccagcctgagaataattacaagaccacccctcctgtgctggacagcgacggcagcttcttcctgtacagcaagctgaccgtggacaagagcagatggcagcagggcaatgtgttcagctgcagcgtgatgcacgaggccctgcacaatcactacacacagaagagcctgagcctgagccctggcaagtgataaggatatcaagatcttgcggccgctcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgaggaagcttaagctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagtctagagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 13 - DNA, 4348 bp, Artificial Sequence; identical to SEQ ID NO: 12
  EXG102-03-2

SEQ ID NO: 14 - DNA, 2755 bp, Artificial Sequence
  EXG102-04
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcgccaccatggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaaggacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatgataaggatatcaagatctacaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 15 - DNA, 2755 bp, Artificial Sequence; identical to SEQ ID NO: 14
  EXG102-05

SEQ ID NO: 16 - DNA, 3002 bp, Artificial Sequence
  EXG102-06
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcgccaccatggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaaggacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatgataaggatatcaagatctataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 17 - DNA, 3179 bp, Artificial Sequence
  EXG102-07
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcgccaccatggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaaggacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaaggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgaacacatgctggagtgataaggatatcaagatctataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 18 - DNA, 2932 bp, Artificial Sequence
  EXG102-08
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcgccaccatggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaaggacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaaggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgaacacatgctggagtgataaggatatcaagatctacaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 19 - DNA, 3937 bp, Artificial Sequence
  EXG102-09
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaacgcgtctcgagagaattcgccaccatggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaagggcggcggcggaggcagcggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgaacacatgctggaggacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatgataagcggccgcaagcttaagagcttaagctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagtctagagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 20 - DNA, 3224 bp, Artificial Sequence
  EXG102-10
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattacgcgtctcgagagaattcgccaccatggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaagggcggcggcggaggcagcggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgaacacatgctggaggacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatgataagcggccgcaagcttaagagatctataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 21 - DNA, 3937 bp, Artificial Sequence
  EXG102-11
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaacgcgtctcgagagaattcgccaccatggtgtcttattgggatactggcgtgctgctctgtgccctcctgagttgcctgctcctgactggttcttcttctgggggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgaacacatgctggagggcggcggcggaggcagctccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaaggacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatgataagcggccgcaagcttaagagcttaagctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagtctagagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 22 - DNA, 2941 bp, Artificial Sequence
  EXG102-12
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcgccaccatggtgtcttattgggatactggcgtgctgctctgtgccctcctgagttgcctgctcctgactggttcttcttctgggggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgaacacatgctggagggcggcggcggaggcagctccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaaggacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatgataagcggccgcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 23 - DNA, 2603 bp, Artificial Sequence
  EXG102-13
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattacgcgtctcgagagaattcgccaccatggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaaggacaaaactcatacctgcccaccttgtccagcaccagagctgctcggaggaccatccgtgggcggcggcggaggcagcggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgaacacatgctggagtgataagcggccgcaagcttaagagatctataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 24 - protein, 5 aa, Artificial Sequence
  Exemplary Peptide Linkers
DGGGS

SEQ ID NO: 25 - protein, 5 aa, Artificial Sequence
  Exemplary Peptide Linkers
TGEKP

SEQ ID NO: 26 - protein, 4 aa, Artificial Sequence
  Exemplary Peptide Linkers
GGRR

SEQ ID NO: 27 - protein, 24 aa, Artificial Sequence
  Exemplary Peptide Linkers
GGGGSGGGGSGGGGGGSGSGGGGS

SEQ ID NO: 28 - protein, 34 aa, Artificial Sequence
  Exemplary Peptide Linkers
GGGGSGGGGSGGGGGGSGSGGGGSGGGGSGGGGS

SEQ ID NO: 29 - protein, 14 aa, Artificial Sequence
  Exemplary Peptide Linkers
EGKSSGSGSESKVD

SEQ ID NO: 30 - protein, 16 aa, Artificial Sequence
  Exemplary Peptide Linkers
KESGSVSSEQLAQFRS

SEQ ID NO: 31 - protein, 8 aa, Artificial Sequence
  Exemplary Peptide Linkers
GGRRGGGS

SEQ ID NO: 32 - protein, 9 aa, Artificial Sequence
  Exemplary Peptide Linkers
LRQRDGERP

SEQ ID NO: 33 - protein, 12 aa, Artificial Sequence
  Exemplary Peptide Linkers
LRQKDGGGSERP

SEQ ID NO: 34 - protein, 16 aa, Artificial Sequence
  Exemplary Peptide Linkers
LRQKDGGGSGGGSERP

SEQ ID NO: 35 - protein, 16 aa, Artificial Sequence
  Exemplary Peptide Linkers
GSTSGSGKPGSGEGST

SEQ ID NO: 36 - protein, 14 aa, Artificial Sequence
  Exemplary Peptide Linkers
GSTSGSGKSSEGKG

SEQ ID NO: 37 - protein, 18 aa, Artificial Sequence
  Exemplary Peptide Linkers
KESGSVSSEQLAQFRSLD

SEQ ID NO: 38 - protein, 2 aa, Artificial Sequence
  Exemplary Peptide Linkers
  MISC_FEATURE (1)..(2): GS can be repeated n times, where n is an integer including, e.g., 1, 2, 3, 4, 5, and 6
GS

SEQ ID NO: 39 - protein, 5 aa, Artificial Sequence
  Exemplary Peptide Linkers
  MISC_FEATURE (1)..(5): GSGGS can be repeated n times, where n is an integer including, e.g., 1, 2, 3, 4, 5, and 6
GSGGS

SEQ ID NO: 40 - protein, 4 aa, Artificial Sequence
  Exemplary Peptide Linkers
  MISC_FEATURE (1)..(4): GGGS can be repeated n times, where n is an integer including, e.g., 1, 2, 3, 4, 5, and 6
GGGS

SEQ ID NO: 41 - protein, 5 aa, Artificial Sequence
  Exemplary Peptide Linkers
  MISC_FEATURE (1)..(5): GGGGS can be repeated n times, where n is an integer including, e.g., 1, 2, 3, 4, 5, and 6
GGGGS

SEQ ID NO: 42 - protein, 6 aa, Artificial Sequence
  Exemplary Peptide Linkers
  MISC_FEATURE (1)..(6): GGGGGS can be repeated n times, where n is an integer including, e.g., 1, 2, 3, 4, 5, and 6
GGGGGS

SEQ ID NO: 43 - protein, 735 aa, Artificial Sequence [sequence omitted]

SEQ ID NO: 44 - protein, 738 aa, Artificial Sequence
  AAV8 (Uniprot Q8JQF8_9VIRU)
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 45 - protein, 736 aa, Artificial Sequence
  AAVrh8 (Uniprot Q808Y3_9VIRU)
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 46 - protein, 736 aa, Artificial Sequence
  AAV9 (Uniprot Q6JC40_9VIRU)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 738 aa, Artificial Sequence
  AAVrh10 (Uniprot Q808W5_9VIRU)
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 48 - protein, 735 aa, Artificial Sequence
  AAV2v
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQGVSKVSADNNNSEFSWTGATKYHLNGRDSLVNPGPAMASHKDDEEKFFPQSGVLIFGKQGSEKTNVDIEKVMITDEEEIRTTNPVATEQYGSVSTNLQSGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 49 - protein, 736 aa, Artificial Sequence
  AAV44-9
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSNMASQARNWVPGPSYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 50 - protein, 228 aa, Artificial Sequence
  Exemplary IgG-Fc (2)
KDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 20 aa, Artificial Sequence
  L1
KFNPLDELEETLYEQFTFQQ

SEQ ID NO: 52 - protein, 57 aa, Artificial Sequence
  Exemlary ABD (2) (2xL1)
GGGGGAQKFNPLDELEETLYEQFTFQQGGGGGGGGKFNPLDELEETLYEQFTFQQLE

SEQ ID NO: 53 - protein, 51 aa, Artificial Sequence
  Exemplary ABD (3) (Con4-L1)
GGGGGAQQEECEWDPWTCEHMGGGGGGGGKFNPLDELEETLYEQFTFQQLE

SEQ ID NO: 54 - protein, 70 aa, Artificial Sequence
  Exemplary ABD (4) (L1-Con4)
GGGGGAQGSGSATGGSGSTASSGSGSATHKFNPLDELEETLYEQFTFQQGGGGGQEECEWDPWTCEHMLE

SEQ ID NO: 55 - protein, 105 aa, Artificial Sequence
  Exemplary Trap C (1) derived from VEGFR-2
VYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVG

SEQ ID NO: 56 - protein, 202 aa, Artificial Sequence
  Exemplary Trap C (2) derived from VEGFR-3
YSMTPPTLNITEESHVIDTGDSLSISCRGQHPLEWAWPGAQEAPATGDKDSEDTGVVRDCEGTDARPYCKVLLLHEVHAQDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITG

SEQ ID NO: 57 - protein, 202 aa, Artificial Sequence
  Exemplary Trap C (3) derived from VEGFR-3
YSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITG

SEQ ID NO: 58 - protein, 634 aa, Artificial Sequence
  Exemplary Polypeptide 5 (as in EXG102-24)
MVSYWDTGVLLCALLSCLLLTGSSSGGGGGGAQQEECEWDPWTCEHMGSGSATGGSGSTASSGSGSATHQEECEWDPWTCEHMLEGGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGGSVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 628 aa, Artificial Sequence
  Exemplary Polypeptide 6 (as in EXG102-25)
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGGSVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGAQQEECEWDPWTCEHMGSGSATGGSGSTASSGSGSATHQEECEWDPWTCEHMLE

SEQ ID NO: 60 - protein, 639 aa, Artificial Sequence
  Exemplary Polypeptide 7 (as in EXG102-26)
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGGSVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGAQGSGSATGGSGSTASSGSGSATHKFNPLDELEETLYEQFTFQQGGGGGQEECEWDPWTCEHMLE

SEQ ID NO: 61 - protein, 731 aa, Artificial Sequence
  Exemplary Polypeptide 8 (as in EXG102-27)
MVSYWDTGVLLCALLSCLLLTGSSSGGGGGGAQQEECEWDPWTCEHMGSGSATGGSGSTASSGSGSATHQEECEWDPWTCEHMLEGGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGGSYSMTPPTLNITEESHVIDTGDSLSISCRGQHPLEWAWPGAQEAPATGDKDSEDTGVVRDCEGTDARPYCKVLLLHEVHAQDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 725 aa, Artificial Sequence
  Exemplary polypeptide 9 (as in EXG102-28)
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGGSYSMTPPTLNITEESHVIDTGDSLSISCRGQHPLEWAWPGAQEAPATGDKDSEDTGVVRDCEGTDARPYCKVLLLHEVHAQDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGAQQEECEWDPWTCEHMGSGSATGGSGSTASSGSGSATHQEECEWDPWTCEHMLE

SEQ ID NO: 63 - protein, 736 aa, Artificial Sequence
  Exemplary polypeptide 10 (as in EXG102-29)
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGGSYSMTPPTLNITEESHVIDTGDSLSISCRGQHPLEWAWPGAQEAPATGDKDSEDTGVVRDCEGTDARPYCKVLLLHEVHAQDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGAQGSGSATGGSGSTASSGSGSATHKFNPLDELEETLYEQFTFQQGGGGGQEECEWDPWTCEHMLE

SEQ ID NO: 64 - protein, 534 aa, Artificial Sequence
  Exemplary polypeptide 11 (as in EXG102-30)
MVSYWDTGVLLCALLSCLLLTGSSSGGGGGGAQGSGSATGGSGSTASSGSGSATHKFNPLDELEETLYEQFTFQQGGGGGQEECEWDPWTCEHMLEGGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 742 aa, Artificial Sequence
  Exemplary polypeptide 12 (as in EXG102-31)
MVSYWDTGVLLCALLSCLLLTGSSSGGGGGGAQGSGSATGGSGSTASSGSGSATHKFNPLDELEETLYEQFTFQQGGGGGQEECEWDPWTCEHMLEGGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGGSYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 736 aa, Artificial Sequence
  Exemplary polypeptide 13 (as in EXG102-32)
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGGSYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGAQGSGSATGGSGSTASSGSGSATHKFNPLDELEETLYEQFTFQQGGGGGQEECEWDPWTCEHMLE

SEQ ID NO: 67 - DNA, 1905 bp, Artificial Sequence
  Nucleic Acid of Exemplary Polypeptide 5 (as in EXG102-24)
atggtgtcttattgggatactggcgtgctgctctgtgccctcctgagttgcctgctcctgactggttcttcttctgggggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgaacacatgctggagggcggcggcggaggcagctccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaagggcggcggcggaggcagcgtgtacgtgcaagactacagaagccccttcatcgctagcgtgagcgatcagcacggcgtggtgtacatcaccgagaacaagaacaagaccgtggtgatcccctgcctgggcagcatcagcaacctgaacgtgagcctgtgcgctagataccccgagaagagattcgtgcccgacggcaacagaatcagctgggacagcaagaagggcttcaccatccctagctacatgatcagctacgccggcatggtgttctgcgaggccaagatcaacgacgagagctatcagagcatcatgtacatcgtggtcgtggtgggcgacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatga

SEQ ID NO: 68 - DNA, 1887 bp, Artificial Sequence
  Nucleic Acid of Exemplary Polypeptide 6 (as in EXG102-25)
atggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaagggcggcggcggaggcagcgtgtacgtgcaagactacagaagccccttcatcgctagcgtgagcgatcagcacggcgtggtgtacatcaccgagaacaagaacaagaccgtggtgatcccctgcctgggcagcatcagcaacctgaacgtgagcctgtgcgctagataccccgagaagagattcgtgcccgacggcaacagaatcagctgggacagcaagaagggcttcaccatccctagctacatgatcagctacgccggcatggtgttctgcgaggccaagatcaacgacgagagctatcagagcatcatgtacatcgtggtcgtggtgggcgacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaaggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgagcacatgctggagtga

SEQ ID NO: 69 - DNA, 1920 bp, Artificial Sequence
  Nucleic Acid of Exemplary Polypeptide 7 (as in EXG102-26)
atggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaagggcggcggcggaggcagcgtgtacgtgcaagactacagaagccccttcatcgctagcgtgagcgatcagcacggcgtggtgtacatcaccgagaacaagaacaagaccgtggtgatcccctgcctgggcagcatcagcaacctgaacgtgagcctgtgcgctagataccccgagaagagattcgtgcccgacggcaacagaatcagctgggacagcaagaagggcttcaccatccctagctacatgatcagctacgccggcatggtgttctgcgaggccaagatcaacgacgagagctatcagagcatcatgtacatcgtggtcgtggtgggcgacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaagggggcgggggcggcgctcaaggcagcgggagcgctaccggcggctccgggagcacagctagcagcggcagcggctccgctacccacaagttcaaccccctggacgagctggaggagaccctctacgagcagttcaccttccaacaaggcgggggcgggggccaagaggagtgcgagtgggacccctggacctgcgagcacatgctggagtga

SEQ ID NO: 70 - DNA, 2196 bp, Artificial Sequence
  Nucleic Acid of Exemplary Polypeptide 8 (as in EXG102-27)
atggtgtcttattgggatactggcgtgctgctctgtgccctcctgagttgcctgctcctgactggttcttcttctgggggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgaacacatgctggagggcggcggcggaggcagctccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaagggcggcggcggaggcagctactccatgacccccccgaccctgaacatcaccgaggagagccacgtgatcgacaccggcgacagcctgagcatcagttgccggggccaacaccccctggaatgggcatggcctggggcacaagaggcccccgcaacgggcgacaaggacagcgaggacaccggcgtggtgagagactgcgagggcaccgacgctagaccctactgcaaggtgctgctgctgcacgaggtgcacgcccaagacaccggtagctacgtgtgctactataagtacatcaaggctagaatcgagggcaccaccgccgctagcagctacgtgtttgtgagagacttcgagcagcccttcatcaacaagcccgacaccctgctggtgaacagaaaggacgccatgtgggtgccctgcctggtgagcatccccggcctgaacgtgaccctgagatctcagagcagcgtgctgtggcccgacggccaagaggtggtgtgggacgacagaagaggcatgctggtgagcacccccctgctgcacgacgccctgtacctgcagtgcgagaccacctggggcgaccaagacttcctgagcaaccccttcctggtgcacatcacaggcgacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatga

SEQ ID NO: 71 - DNA, 2178 bp, Artificial Sequence
  Nucleic Acid of Exemplary polypeptide 9 (as in EXG102-28)
atggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaagggcggcggcggaggcagctactccatgacccccccgaccctgaacatcaccgaggagagccacgtgatcgacaccggcgacagcctgagcatcagttgccggggccaacaccccctggaatgggcatggcctggggcacaagaggcccccgcaacgggcgacaaggacagcgaggacaccggcgtggtgagagactgcgagggcaccgacgctagaccctactgcaaggtgctgctgctgcacgaggtgcacgcccaagacaccggtagctacgtgtgctactataagtacatcaaggctagaatcgagggcaccaccgccgctagcagctacgtgtttgtgagagacttcgagcagcccttcatcaacaagcccgacaccctgctggtgaacagaaaggacgccatgtgggtgccctgcctggtgagcatccccggcctgaacgtgaccctgagatctcagagcagcgtgctgtggcccgacggccaagaggtggtgtgggacgacagaagaggcatgctggtgagcacccccctgctgcacgacgccctgtacctgcagtgcgagaccacctggggcgaccaagacttcctgagcaaccccttcctggtgcacatcaccggcgacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaaggcggcggcggaggcgcccagcaagaagagtgcgagtgggatccctggacctgcgagcacatgggatccggcagcgccaccggaggatccggaagcaccgcctccagcggctccggcagcgccacccaccaggaggagtgtgagtgggacccctggacctgcgaacacatgctggagtga

SEQ ID NO: 72 - DNA, 2211 bp, Artificial Sequence
  Nucleic Acid of Exemplary polypeptide 10 (as in EXG102-29)
atggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaagggcggcggcggaggcagctactccatgacccccccgaccctgaacatcaccgaggagagccacgtgatcgacaccggcgacagcctgagcatcagttgccggggccaacaccccctggaatgggcatggcctggggcacaagaggcccccgcaacgggcgacaaggacagcgaggacaccggcgtggtgagagactgcgagggcaccgacgctagaccctactgcaaggtgctgctgctgcacgaggtgcacgcccaagacaccggtagctacgtgtgctactataagtacatcaaggctagaatcgagggcaccaccgccgctagcagctacgtgtttgtgagagacttcgagcagcccttcatcaacaagcccgacaccctgctggtgaacagaaaggacgccatgtgggtgccctgcctggtgagcatccccggcctgaacgtgaccctgagatctcagagcagcgtgctgtggcccgacggccaagaggtggtgtgggacgacagaagaggcatgctggtgagcacccccctgctgcacgacgccctgtacctgcagtgcgagaccacctggggcgaccaagacttcctgagcaaccccttcctggtgcacatcaccggcgacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaagggggcgggggcggcgctcaaggcagcgggagcgctaccggcggctccgggagcacagctagcagcggcagcggctccgctacccacaagttcaaccccctggacgagctggaggagaccctctacgagcagttcaccttccaacaaggcgggggcgggggccaagaggagtgcgagtgggacccctggacctgcgagcacatgctggagtga

SEQ ID NO: 73 - DNA, 1605 bp, Artificial Sequence
  Nucleic Acid of Exemplary polypeptide 11 (as in EXG102-30)
atggtgtcttattgggatactggcgtgctgctctgtgccctcctgagttgcctgctcctgactggttcttcttctggggggggcgggggcggcgctcaaggcagcgggagcgctaccggcggctccgggagcacagctagcagcggcagcggctccgctacccacaagttcaaccccctggacgagctggaggagaccctctacgagcagttcaccttccaacaaggcgggggcgggggccaagaggagtgcgagtgggacccctggacctgcgagcacatgctggagggcggcggcggaggcagctccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaaggacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatga

SEQ ID NO: 74 - DNA, 2229 bp, Artificial Sequence
  Nucleic Acid of Exemplary polypeptide 12 (as in EXG102-31)
atggtgtcttattgggatactggcgtgctgctctgtgccctcctgagttgcctgctcctgactggttcttcttctggggggggcgggggcggcgctcaaggcagcgggagcgctaccggcggctccgggagcacagctagcagcggcagcggctccgctacccacaagttcaaccccctggacgagctggaggagaccctctacgagcagttcaccttccaacaaggcgggggcgggggccaagaggagtgcgagtgggacccctggacctgcgagcacatgctggagggcggcggcggaggcagctccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaagggcggcggcggaggcagctactccatgacccccccgaccctgaacatcaccgaggagagccacgtgatcgacaccggcgacagcctgagcatcagttgccggggccaacaccccctggaatgggcatggcctggggcacaagaggcccccgcaacgggcgacaaggacagcgaggacaccggcgtggtgagagactgcgagggcaccgacgctagaccctactgcaaggtgctgctgctgcacgaggtgcacgccaacgacaccggtagctacgtgtgctactataagtacatcaaggctagaatcgagggcaccaccgccgctagcagctacgtgtttgtgagagacttcgagcagcccttcatcaacaagcccgacaccctgctggtgaacagaaaggacgccatgtgggtgccctgcctggtgagcatccccggcctgaacgtgaccctgagatctcagagcagcgtgctgtggcccgacggccaagaggtggtgtgggacgacagaagaggcatgctggtgagcacccccctgctgcacgacgccctgtacctgcagtgcgagaccacctggggcgaccaagacttcctgagcaaccccttcctggtgcacatcacaggcgacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaatga

SEQ ID NO: 75 - DNA, 2211 bp, Artificial Sequence
  Nucleic Acid of Exemplary polypeptide 13 (as in EXG102-32)
atggtgtcttattgggatactggcgtgctgctctgtgccctcctgagttgcctgctcctgactggttcttcttctgggtccgatactgggcgccccttcgtggagatgtactccgagatccctgaaatcattcacatgactgagggtcgggaactggtcatcccatgccgcgtgacctctcccaacattactgtgaccctgaagaaattccctctggacaccctcatcccagatgggaagaggatcatttgggactcaagaaagggttttatcatcagcaacgctacatacaaggagattggcctgctcacctgcgaagcaacagtgaacggacacctgtacaagactaattatctcacccatagacagacaaacactatcattgatgtggtcctgtcaccaagccacggcatcgagctcagcgtcggtgaaaagctggtgctcaattgtacagcccggactgagctgaacgtgggcattgacttcaattgggaataccccagctccaagcaccagcataagaaactggtgaaccgcgatctcaaaacccagtccggatctgagatgaagaaatttctgagcaccctcacaatcgacggcgtgacacgatccgatcagggactgtatacttgcgccgcttctagtggcctgatgaccaagaaaaatagcacattcgtcagggtgcacgaaaagggcggcggcggaggcagctactccatgacccccccgaccctgaacatcaccgaggagagccacgtgatcgacaccggcgacagcctgagcatcagttgccggggccaacaccccctggaatgggcatggcctggggcacaagaggcccccgcaacgggcgacaaggacagcgaggacaccggcgtggtgagagactgcgagggcaccgacgctagaccctactgcaaggtgctgctgctgcacgaggtgcacgccaacgacaccggtagctacgtgtgctactataagtacatcaaggctagaatcgagggcaccaccgccgctagcagctacgtgtttgtgagagacttcgagcagcccttcatcaacaagcccgacaccctgctggtgaacagaaaggacgccatgtgggtgccctgcctggtgagcatccccggcctgaacgtgaccctgagatctcagagcagcgtgctgtggcccgacggccaagaggtggtgtgggacgacagaagaggcatgctggtgagcacccccctgctgcacgacgccctgtacctgcagtgcgagaccacctggggcgaccaagacttcctgagcaaccccttcctggtgcacatcacaggcgacaaaactcatacctgcccaccttgtccagcaccagagctgctcggaggaccatccgtgttcctgtttccacccaagcccaaagatactctgatgatttcacgcacacccgaagtcacttgcgtggtcgtggacgtgtcccacgaggaccccgaagtcaagtttaactggtacgtggacggcgtcgaggtgcataatgctaagacaaaaccccgagaggaacagtacaactctacctatagggtcgtgagtgtcctgacagtgctccaccaggattggctgaacggaaaggagtataagtgcaaagtgtctaataaggcactgcctgccccaatcgagaaaacaattagtaaggccaaagggcagcccagagaacctcaggtgtacactctgcctccatctcgggacgagctcactaagaaccaggtcagtctgacctgtctcgtgaaagggttctatcctagtgatatcgctgtggagtgggaatcaaatggtcagccagagaacaattacaagaccacaccccctgtcctggacagcgatggctccttctttctgtattccaagctcaccgtggacaaatctcgatggcagcagggaaacgtctttagttgttcagtgatgcacgaagccctccataaccactacactcagaaaagcctcagcctcagccctgggaaagggggcgggggcggcgctcaaggcagcgggagcgctaccggcggctccgggagcacagctagcagcggcagcggctccgctacccacaagttcaaccccctggacgagctggaggagaccctctacgagcagttcaccttccaacaaggcgggggcgggggccaagaggagtgcgagtgggacccctggacctgcgagcacatgctggagtga